(19) United States
(12) Patent Application Publication
(10) Pub. No.: US 2014/0107697 A1
(43) Pub. Date: Apr. 17, 2014

(54) Clamping Forceps and Associated Methods

(71) Applicants: Paras Patani, New York, NY (US); Sanjeev Kaul, Troy, MI (US); Aaron Asch, Hamden, CT (US); Rahman Nabulsi, Somerville, MA (US); James Brown, Lynchburg, VA (US)

(72) Inventors: Paras Patani, New York, NY (US); Sanjeev Kaul, Troy, MI (US); Aaron Asch, Hamden, CT (US); Rahman Nabulsi, Somerville, MA (US); James Brown, Lynchburg, VA (US)

(73) Assignee: CASTLE SURGICAL, INC., New York, NY (US)

(21) Appl. No.: 14/140,193

(22) Filed: Dec. 24, 2013

Related U.S. Application Data
(63) Continuation-in-part of application No. PCT/US2012/043940, filed on Jun. 25, 2012.

(51) Int. Cl. A61B 17/28

(52) U.S. Cl. CPC A61B 17/2833
USPC 606/208; 606/205

(57) ABSTRACT

Clamping forceps and associated methods are provided that generally include a head section including first and second clamp members configured to cooperatively clamp so as to at least partially encircle a tumor or other structure, an elongated body section in cooperation with the head section, and a clamping mechanism at least partially movably mounted with respect to the elongated body section. The clamping forceps may include a control mechanism for variably controlling the clamping force, at least one sensor mounted with respect to the clamp member(s), means for fixating the clamp member(s) relative to tissue, the first and second clamp members structured to define a variable perimeter extent, and a clamping mechanism which accommodates variable rates of clamping action. Actuation of the clamping mechanism generally maintains substantially parallel actuation of the first and second clamp members with respect to the tumor or the other anatomical structure. Methods for use of the clamping forceps may include introducing the head section to the surgical site through an incision; positioning a trocar port at the surgical site; introducing at least a portion of the elongated body section to the surgical site through the trocar port; intra-corporeally connecting the head section with respect to the elongated body section; and extra-corporeally actuating the clamping mechanism so as to actuate the first and second clamp members into a clamping position around the tumor or other anatomical structure.

### RELATED APPLICATIONS

This application is based on and claims priority benefit from U.S. Provisional Application No. 61/501,198, filed Jun. 25, 2011, and PCT/US2012/043940, filed Jun. 25, 2012. The entire contents of the foregoing provisional patent application and PCT application are incorporated herein by reference.

### TECHNICAL FIELD

The present disclosure relates to clamping forceps and associated methods and, in particular, to clamping forceps which permit clamping of a specific region of an organ and thereby improve surgical conditions.

### BACKGROUND

Numerous organs in the human body, e.g., the kidney, liver, and the like, are extremely sensitive to ischemia during surgical operations where blood vessels to the organ are clamped. Ischemia is the restriction in blood supply resulting in damage and/or dysfunction of tissue. The degree of ischemia is often measured by warm ischemia time (WIT), which is the amount of time blood flow is cut off from the organ in order to perform an operation.

For example, partial nephrectomy procedures are performed to remove tumors from a kidney. In general, during a partial nephrectomy procedure, blood vessels, e.g., the renal artery, and the like, leading to the kidney are clamped to prevent blood flow from reaching the kidney during the surgical procedure. According to the latest available research, during a partial nephrectomy procedure, the kidney can survive for a WIT of approximately 20 minutes before it degenerates significantly and the risk for long-term damage and life-long patient complications increase dramatically. (See, e.g., Lane, B. R. et al., Factors predicting renal functional outcome after partial nephrectomy, The Journal of Urology, 180(6), p. 2363-2369 (2008); Becker, F. et al., Assessing the Impact of Ischaemia Time During Partial Nephrectomy, European Urology, 56(4), p. 625-635 (2009); and Huang, W. C. et al., Chronic kidney disease after nephrectomy in patients with renal cortical tumors: a retrospective cohort study, The Lancet Oncology, 7(9), p. 735-740 (2006)). Indeed, some surgeons suggest that every minute that the kidney is without blood flow progressively leads to greater ischemic degeneration. (See, e.g., Thompson, R. H. et al., Every minute counts when the renal hilum is clamped during partial nephrectomy, European Urology, 58(3), p. 340-345 (2010); and Patel, A. R. et al., Warm ischemia less than 30 minutes is not necessarily safe during partial nephrectomy: Every minute matters, Urologic Oncology: Seminars and Original Investigations, Vol. 29, p. 826-828 (2011). After approximately 30 minutes of WIT, many surgeons consider it too late and the risks too high to save the kidney and will subsequently resect and remove the entire kidney from the patient. Therefore, some surgeons have proposed that the non-ischemic approach is generally best for long-term renal function. (See, e.g., Aron, M. et al., A Nonischemic Approach to Partial Nephrectomy is Optimal, The Journal of Urology, 187(2), p. 387-388 (2012)). The consideration of WIT typically requires surgeons to rush during surgical procedures to prevent damage to organs, which may reduce the precision and/or care taken during the surgical procedure. The hastened surgical procedure may further increase complications and/or tumor recurrence in patients, thus requiring further surgical procedures and/or medication administration to minimize patient discomfort.

Thus, a need exists for clamping forceps and associated methods for restricting and/or preventing blood flow to specific areas of an organ and/or tissue during surgical operations, while maintaining blood flow to other areas of an organ and/or tissue. Further, clamping forceps and associated methods are needed that are effective for clamping thick-body tissue, as opposed to thin-body clamping (e.g., vascular clamping devices and methodologies). Still further, a need exists for clamping forceps and associated methods which provide additional time for surgeons to perform surgical procedures, thereby increasing the precision and/or care taken during the surgical procedures and reducing the occurrence of complications and/or tumor recurrence in patients. These and other needs are addressed by the clamping forceps and associated methods of the present disclosure.

### SUMMARY

In accordance with embodiments of the present disclosure, exemplary clamping forceps and associated methods are disclosed that generally include a head section including a first clamp member and a second clamp member configured and dimensioned to cooperatively clamp, e.g., so as to at least partially encircle a tumor or other structure. The exemplary clamping forceps generally includes an elongated body section in cooperation with the head section and a clamping mechanism that is at least partially movably mounted with respect to the elongated body section. Further, exemplary clamping forceps according to the present disclosure may include a control mechanism for variably controlling a clamping force applied with respect to at least a portion of at least one of the first clamp member and the second clamp member. The variable clamping force applied with respect to at least one of the first clamp member and the second clamp member is generally effective to permit controlled blood flow therethrough, e.g., to a tumor positioned relative to the first clamp member and the second clamp member, based on a controlled reduced clamping force in a region of controlled blood flow. The controlled blood flow of the exemplary clamping forceps is typically at least one of, e.g., an intermittent blood flow, a moderated blood flow, a localized blood flow, a fully unrestricted blood flow, a fully restricted blood flow, and the like.

In accordance with embodiments of the present disclosure, exemplary methods of clamping are also provided. The exemplary methods generally include introducing a clamping forceps according to the present disclosure to a surgical site. For example, the clamping forceps may include a first clamp member and a second clamp member configured and dimensioned to at least partially encircle a tumor or other structure. The exemplary clamping forceps may also include a control mechanism for variably controlling a clamping force applied with respect to at least a portion of at least one of the first clamp member and the second clamp member. The exemplary methods may also generally include positioning the first clamp member and the second member so as to at least partially encircle a tumor or other structure. The disclosed method may also further generally include variably controlling the clamping force applied with respect to at least a portion of at least one of the first clamp member- and the second clamp member to permit controlled blood flow with respect thereto, e.g., to a tumor positioned relative to the first clamp member and the second clamp member, based on a controlled reduced clamping force in a region of controlled blood flow. The controlled blood flow is typically at least one of e.g., an intermittent blood flow, a moderated blood flow, a localized blood flow, a fully unrestricted blood flow, a fully restricted blood flow, and the like.

Exemplary methods according to the present disclosure may also generally include implementing a duty cycle to variably control the clamping force applied with respect to at least a portion of at least one of the first clamp member and the second clamp member.

In accordance with another embodiment of the present disclosure, exemplary clamping forceps generally includes a head section including a first clamp member and a second clamp member configured and dimensioned to cooperatively clamp, e.g., so as to at least partially encircle a tumor or other structure. The exemplary clamping forceps also generally include an elongated body section in cooperation with respect to the head section and a clamping mechanism movably mounted at least in part with respect to the elongated body. In general, the exemplary clamping forceps includes at least one sensor mounted with respect to at least one of the first clamp member and the second clamp member. The at least one sensor is typically effective to generate signals related to at least one anatomical parameter. Distal movement of the clamping mechanism (at least in part) relative to the elongated body section functions to move the first clamp member and the second clamp member into a clamping orientation relative to the tumor.

The at least one anatomical parameter can be at least one of, e.g., a blood flow to a tumor and/or tissue surrounding the surgical site, a contour and/or an image of at least a portion of a tumor, an organ, and/or a surrounding tissue, an oxygenation (healthiness) of tissue, a blood vessel location, a tissue and/or disease composition, a proximity to surrounding tissue, a tissue density, a tissue biochemistry, a biomaterial composition, information for application of perfusion and/or therapeutic drugs, and the like. In some exemplary embodiments, the at least one anatomical parameter can be, e.g., a feedback signal from at least one sensor, such as, for example, a strain gauge, relating to at least one of a clamping force and a position of the exemplary clamping forceps. The exemplary clamping forceps generally further include a means for receiving at least one of a visual feedback, an audio feedback, a position feedback, and/or a sensor feedback of the signals related to the at least one anatomical parameter. The visual feedback, the position feedback, and/or the audio feedback typically provide a real-time feedback. In particular, the visual feedback typically includes at least one of, e.g., a flow/no flow light-emitting diode (LED), a plurality of LEDs indicating at least one of a flow level, imaging, a temperature, water content, and a tissue composition, a liquid crystal display (LCD), a light-emitting diode (LED) display, a charged-coupled device (CCD), and the like. Further, the at least one sensor can be, e.g., an ultrasound sensor, a Doppler ultrasound sensor, a pulse oximetry sensor, an infrared sensor, a light transmitter sensor, an IR sensor (an infrared proximity sensor), a stress/strain sensor for tissue stiffness and/or strength, and/or other feedback that may be needed for a physician to provide appropriate and accurate therapy at the surgical site, and the like.

In accordance with embodiments of the present disclosure, exemplary methods of clamping with clamping forceps are also provided that generally include introducing the clamping forceps to a surgical site. The exemplary clamping forceps may generally include a first clamp member and a second clamp member configured and dimensioned to at least partially encircle the tumor or other structure. In general, the exemplary clamping forceps may include at least one sensor mounted with respect to at least one of the first clamp member and the second clamp member. The at least one sensor is typically effective to generate signals related to at least one anatomical parameter. The exemplary methods may generally include positioning the first clamp member and the second clamp member so as to at least partially encircle a tumor and sensing the at least one anatomical parameter based on at least one signal generated by the at least one sensor. The exemplary methods may further generally include taking clinical action based at least in part upon the sensed at least one anatomical parameter.

Taking clinical action generally may further include at least one of adjusting a clamping force delivered with respect to the first clamp member and the second clamp member, and adjusting a clamping position with respect to the first clamp member and the second clamp member based on the sensed at least one anatomical parameter. The at least one anatomical parameter can be at least one of, e.g., a blood flow to a tumor and/or tissue surrounding the surgical site, a contour and/or an image of at least a portion of a tumor, an organ, and/or a surrounding tissue, an oxygenation (healthiness) of tissue, a blood vessel location, a tissue and/or disease composition, a proximity to surrounding tissue, a tissue density, a tissue biochemistry, a biomaterial composition, information for application of perfusion and/or therapeutic drugs, and the like. The exemplary methods may generally include at least one of, e.g., restricting or preventing blood flow to the tumor, restricting or preventing blood flow to the tissue, generating the contour of at least a portion of the tumor, generating the contour of at least a portion of the organ, generating the contour of at least a portion of the tissue, generating the image of at least a portion of the tumor, generating the image of at least a portion of the organ, generating the image of at least a portion of the tissue, sensing the oxygenation of tissue, detecting the blood vessel location, detecting the tissue composition, detecting the disease composition, detecting the proximity to surrounding tissue, detecting a tissue density, detecting a tissue biochemistry, and detecting a biomaterial composition, and the like.

In accordance with another embodiment of the present disclosure, exemplary clamping forceps are provided that generally include a head section including a first clamp member and a second clamp member configured and dimensioned to cooperatively clamp, e.g., so as to at least partially encircle a tumor or other structure. The exemplary clamping forceps also generally include an elongated body section in cooperation with respect to the head section and a clamping mechanism at least partially movably mounted with respect to the elongated body section. In general, the exemplary clamping forceps may include means for fixating at least one of the first clamp member and the second clamp member relative to tissue. Distal movement of the clamping mechanism (at least in part) relative to the elongated body section typically functions to move the first clamp member and the second clamp member into a clamping orientation, e.g., relative to a tumor.

The means for fixating at least one of the first clamp member and the second clamp member relative to tissue includes at least one of, e.g., a suction mechanism, a textured surface, a coated surface, and the like, deployed on a clamping surface. The suction mechanism generally includes at least one of the first clamp member and the second clamp member defining at least one spaced opening in communication with a source of negative pressure flow, e.g., a vacuum, suction, and the like, and optionally in communication with a source of positive pressure flow. The body section typically includes at least one conduit, e.g., a tube, a passage, a cavity, a line, a lumen, a hollow interior, and the like, in communication with the at least one spaced opening. The first clamp member and the second clamp member generally also include at least one conduit, e.g., a tube, a passage, a cavity, a line, a lumen, a hollow interior, and the like, for delivery of the negative pressure flow and/or the positive pressure flow with respect to the at least one spaced opening. Delivery of a negative pressure flow to the at least one spaced opening is generally effective to draw tissue into the at least one spaced opening. Delivery of a positive pressure flow to the at least one spaced opening is generally effective to push out the drawn tissue from the at least one spaced opening. The textured surface generally includes at least one of, e.g., one or more spikes, one or more ridges, an alternative tissue-gripping mechanism, and the like. The coated surface generally includes, e.g., a hydrophilic coating, a hydrophobic coating, and the like.

In accordance with embodiments of the present disclosure, exemplary methods of clamping with a clamping forceps are also provided that generally include introducing the clamping forceps to a surgical site. The exemplary clamping forceps generally include a first clamp member and a second clamp member configured and dimensioned to at least partially encircle a tumor or other structure. In general, the exemplary clamping forceps further include means for fixating at least one of the first clamp member and the second clamp member relative to a tissue. The exemplary methods typically include positioning the first clamp member and the second clamp member so as to at least partially encircle the tumor or other structure and fixating at least one of the first clamp member and the second clamp member relative to the tissue.

The means for fixating at least one of the first clamp member and the second clamp member relative to the tissue typically include at least one of, e.g., a suction mechanism, a textured surface, a coated surface, and the like, deployed on a clamping surface. The suction mechanism generally includes at least one of the first clamp member and the second clamp member defining at least one spaced opening in communication with a source of negative pressure flow, e.g., a vacuum, suction, and the like, and in communication with a source of positive pressure flow. The exemplary methods generally include actuating the at least one spaced opening into communication with the source of negative pressure flow to draw tissue into the at least one spaced opening. The exemplary methods generally further include actuating the at least one spaced opening into communication with the source of positive pressure flow to push out the drawn tissue from the at least one spaced opening.

In accordance with another embodiment of the present disclosure, exemplary clamping forceps generally include a head section including a first clamp member and a second clamp member configured and dimensioned to cooperatively clamp, e.g., so as to at least partially encircle a tumor or other structure. The exemplary clamping forceps generally further include an elongated body section in cooperation with respect to the head section and a clamping mechanism at least partially movably mounted with respect to the elongated body section. The first clamp member and the second clamp member are generally structured so as to define a variable perimeter extent. The variable perimeter extent permits variability and/or adjustment in the degree to which a tumor or other structure is encircled by the first clamp member and the second clamp member. Further, the first clamp member and the second clamp member can be axially rotatable with respect to the elongated body section.

In accordance with embodiments of the present disclosure, exemplary methods of clamping with a clamping forceps are also provided, generally including introducing the clamping forceps to a surgical site. The exemplary clamping forceps generally include a first clamp member and a second clamp member configured and dimensioned to at least partially encircle a tumor or other structure. The first clamp member and the second clamp member generally define a variable perimeter extent that permits variability and/or adjustment in the degree to which the tumor or other structure is encircled by the first clamp member and the second clamp member. The exemplary method generally includes positioning the first clamp member and the second clamp member so as to at least partially encircle the tumor or other structure. Further, the exemplary method generally includes adjusting the variable perimeter extent of the first clamp member and the second clamp member. In addition, the exemplary method can include axially rotating the first clamp member and the second clamp member with respect to the elongated body section.

In accordance with another embodiment of the present disclosure, exemplary clamping forceps generally include a head section including a first clamp member and a second clamp member configured and dimensioned to cooperatively clamp so as to at least partially encircle a tumor or other structure. The exemplary clamping forceps generally further includes an elongated body section in cooperation with respect to the head section and a clamping mechanism at least partially movably mounted with respect to the elongated body section. The clamping mechanism generally accommodates variable rates of clamping action by at least one of, e.g., a worm gear, a surgeon actuated toggle, a response to a sensor reading, a variably sized gearing mechanism, and the like. The variable rates of clamping action generally transition the clamping mechanism between a gross adjustment clamping action and a fine adjustment clamping action. Further, the variable rates of clamping action generally include at least one of, e.g., a substantially continuous clamping action, a substantially one centimeter increment clamping action, a substantially one millimeter increment clamping action, a substantially one hundred micrometer increment clamping action, and the like. Although described herein as a substantially one centimeter, one millimeter, and/or one hundred micrometer increment clamping action, in some exemplary embodiments, it should be understood that the clamping action can be, e.g., one, two, three, four, five, six, and the like, centimeter and/or millimeter increments, and/or, e.g., one hundred, two hundred, three hundred, four hundred, five hundred, six hundred, and the like, micrometer increments.

In accordance with embodiments of the present disclosure, exemplary methods of clamping with a clamping forceps are also provided, generally including introducing the clamping forceps to a surgical site. The exemplary clamping forceps generally includes a first clamp member and a second clamp member configured and dimensioned to at least partially encircle the tumor or other structure. In general, the exemplary clamping forceps also include an elongated body section in cooperation with respect to the first clamp member and the second clamp member and a clamping mechanism at least partially movably mounted with respect to the elongated body section. The exemplary methods further include adjusting the movement of the clamping mechanism at variable rates of clamping action. The variable rates of clamping action of the clamping mechanism are generally accommodated by at least one of e.g., a worm gear, a surgeon actuated toggle, a response to a sensor reading, a variably sized gearing mechanism, and the like. The variable rates of clamping action typically transition the clamping mechanism between a gross adjustment clamping action and a fine adjustment clamping action.

In accordance with another embodiment of the present disclosure, exemplary clamping forceps generally include a head section including a first clamp member and a second clamp member configured and dimensioned to cooperatively clamp so as to at least partially encircle a tumor or other structure. The exemplary clamping forceps generally also include an elongated body section in cooperation with respect to the head section and a clamping mechanism at least partially movably mounted with respect to the elongated body section. Further, the exemplary clamping forceps generally include a control mechanism for automatically adjusting a clamping force applied with respect to at least a portion of at least one of the first clamp member and the second clamp member. The control mechanism generally maintains a substantially uniform clamping force throughout a surgical procedure against, e.g., the tumor, other structure, an organ, tissue, and the like.

In accordance with embodiments of the present disclosure, exemplary methods of clamping with a clamping forceps are also provided, generally including introducing the clamping forceps to a surgical site. The exemplary clamping forceps generally includes a first clamp member and a second clamp member configured and dimensioned to at least partially encircle a tumor or other structure. In general, the exemplary clamping forceps includes a control mechanism for automatically adjusting a clamping force applied with respect to at least a portion of at least one of the first clamp member and the second clamp member. The exemplary methods generally include positioning the first clamp member and the second clamp member so as to at least partially encircle the tumor or other structure. Further, the exemplary methods generally include clamping the first clamp member and the second clamp member around the tumor or other structure at a desired clamping force. In general, the exemplary methods include automatically adjusting the clamping force throughout a surgical procedure to maintain a substantially uniform clamping force against, e.g., the tumor, other structure, an organ, tissue, and the like.

In accordance with another embodiment of the present disclosure, exemplary clamping forceps generally include a head section including a first clamp member and a second clamp member configured and dimensioned to cooperatively clamp so as to at least partially encircle a tumor or other structure. The exemplary clamping forceps generally include an elongated body section in cooperation with respect to the head section and a clamping mechanism at least partially movably mounted with respect to the elongated body section. In general, the exemplary clamping forceps include a rotatable shaft disposed within the elongated body section. The rotatable shaft typically permits an angular rotation of a handle section relative to the elongated body section.

In accordance with embodiments of the present disclosure, exemplary methods of clamping with a clamping forceps are also provided, generally including introducing the clamping forceps to a surgical site. The exemplary clamping forceps generally include a first clamp member and a second clamp member configured and dimensioned to at least partially encircle a tumor or other structure. The exemplary clamping forceps generally further include a rotatable shaft disposed within an elongated body section. The exemplary methods generally include positioning the first clamp member and the second clamp member so as to at least partially encircle the tumor or other structure. The exemplary methods generally further include clamping the first clamp member and the second clamp member around the tumor or other structure. Further, the exemplary methods generally include angularly rotating a handle section relative to the elongated body section.

In accordance with another embodiment of the present disclosure, exemplary clamping forceps generally include a head section including a first clamp member and a second clamp member configured and dimensioned to cooperatively clamp so as to at least partially encircle a tumor or other structure. The exemplary clamping forceps generally include an elongated body section in cooperation with respect to the head section and a clamping mechanism at least partially movably mounted with respect to the elongated body section. The first clamp member and the second clamp member are generally detachable relative to the head section. In some exemplary embodiments, the head section can generally be detachable relative to the elongated body section. Detachment of the head section advantageously facilitates hybrid surgical procedures, whereby benefits of laparoscopic or minimally invasive surgical procedures and benefits of open surgical procedures are achieved in conjunction with introduction of the clamping members to the surgical site. Thus, for example, benefits associated with an open surgical procedure, such as an ability to utilize mechanically solid first and second clamping members without a need for folding linkages or other structural accommodations to facilitate introduction through a trocar port/cannula, are achieved in conjunction with an introduction of the clamping members through a laparoscopic incision.

In accordance with embodiments of the present disclosure, exemplary methods of clamping with a clamping forceps are also provided, generally including introducing the clamping forceps to a surgical site. The exemplary clamping forceps generally include a first clamp member and a second clamp member configured and dimensioned to at least partially encircle a tumor or other structure. Further, the exemplary clamping forceps generally include an elongated body section in cooperation with respect to the first clamp member and the second clamp member. The exemplary methods generally include positioning the first clamp member and the second clamp member so as to at least partially encircle the tumor or other structure. In general, the exemplary methods include clamping the first clamp member and the second clamp member around the tumor or other structure and unclamping the first clamp member and the second clamp member during a surgical procedure. Further, the exemplary methods generally include detaching the first clamp member and the second clamp member from the head section. In some embodiments, the exemplary methods generally include detaching the head section form the elongated body section.

In accordance with another embodiment of the present disclosure, exemplary clamping forceps generally include a head section including a first clamp member and a second clamp member configured and dimensioned to cooperatively clamp so as to at least partially encircle a tumor or other structure. The exemplary clamping forceps generally further include an elongated body section in cooperation with respect to the head section and a clamping mechanism at least partially movably mounted with respect to the elongated body section. The first clamp member and the second clamp member generally define topographically variable clamping surfaces so as to conform to, e.g., a topography of tissue surrounding a tumor or other structure, a topography of a tumor, and the like.

In accordance with embodiments of the present disclosure, exemplary methods of clamping with a clamping forceps are also provided, generally including introducing the clamping forceps to a surgical site. The exemplary clamping forceps generally include a first clamp member and a second clamp member configured and dimensioned to at least partially encircle a tumor or other structure. The exemplary methods generally include positioning the first clamp member and the second clamp member so as to at least partially encircle the tumor or other structure. Further, the exemplary methods generally include topographically varying clamping surfaces of the first clamp member and the second clamp member based on, e.g., a topography of tissue surrounding the tumor or other structure, a topography of a tumor, and the like.

In accordance with embodiments of the present disclosure, exemplary clamping forceps generally include a head section including a first clamp member and a second clamp member configured and dimensioned to cooperatively clamp so as to at least partially encircle a tumor or other structure. The exemplary clamping forceps generally further include an elongated body section in cooperation with respect to the head section and a clamping mechanism at least partially movably mounted with respect to the elongated body section. At least one of the first clamp member and the second clamp member is generally defined by a plurality of interconnected linkages. The first and second clamp members generally define at least one of a uniform perimeter configuration and a variable perimeter configuration. A clamping surface of at least one of the first clamp member and the second lamp member is generally, e.g., substantially curved, straight, angled, variable, and the like.

In accordance with embodiments of the present disclosure, exemplary methods of clamping with a clamping forceps are also provided, generally including introducing the clamping forceps to a surgical site. The exemplary clamping forceps generally include a first clamp member and a second clamp member configured and dimensioned to at least partially encircle a tumor or other structure. At least one of the first clamp member and the second clamp member generally defines a plurality of interconnected linkages. The exemplary methods generally include positioning the first clamp member and the second clamp member so as to at least partially encircle a tumor or other structure. Further, the exemplary methods generally include varying a perimeter extent of at least one of the first clamp member and the second clamp member. In general, the exemplary methods further include topographically varying a clamping surface of at least one of the first clamp member and the second clamp member.

In accordance with embodiments of the present disclosure, exemplary clamping forceps are provided that generally include a head section including a first clamp member and a second clamp member configured and dimensioned to cooperatively clamp so as to at least partially encircle a tumor or other structure. The exemplary clamping forceps generally further include an elongated body section in cooperation with respect to the head section and a clamping mechanism at least partially movably mounted with respect to the elongated body section. In general, the exemplary clamping forceps also includes a rotatable shaft disposed within the elongated body section. The rotatable shaft generally permits an angular rotation of at least one of the first clamp member and the second clamp member relative to the elongated body section.

In accordance with embodiments of the present disclosure, exemplary methods of clamping with a clamping forceps are also provided, generally including introducing the clamping forceps to a surgical site. The exemplary clamping forceps generally include a first clamp member and a second clamp member configured and dimensioned to at least partially encircle a tumor or other structure. The exemplary clamping forceps generally further include a rotatable shaft disposed within an elongated body section. The exemplary methods generally include positioning the first clamp member and the second clamp member so as to at least partially encircle the tumor or other structure. The exemplary methods generally further include clamping the first clamp member and the second clamp member around the tumor or other structure. In general, the exemplary methods further include angularly rotating at least one of the first clamp member and the second clamp member relative to the elongated body section.

In some embodiments of the present disclosure, a perimeter of the first clamp member and the second clamp member of the exemplary clamping forceps can define at least one of, e.g., a circular configuration, an ovular configuration, a polygonal configuration, a variable configuration, a C-shaped configuration, a J-shaped configuration, an L-shaped configuration, a malleable configuration, a topographically contoured configuration, and the like. The first clamp member generally defines a first clamping surface and the second clamp member generally defines a second clamping surface. The first clamping surface and the second clamping surface can be substantially, e.g., curved, straight, angled, variable, and the like. The exemplary clamping forceps can further include at least one of, e.g., a textured surface, a coated surface, and the like, deployed on the first clamping surface and/or the second clamping surface. The coated surface can be, e.g., a hydrophobic, a hydrophilic, a therapeutic, and the like, surface. In addition, the first clamp member and the second clamp member of the exemplary clamping forceps can be defined by at least one of, e.g., a solid clamp member, a plurality of interconnected linkages, a malleable clamp member, and the like.

In some embodiments of the present disclosure, the exemplary clamping forceps can include means for providing a therapeutic treatment to at least one of, e.g., a tumor, tissue surrounding a tumor, and the like. The therapeutic treatment can be at least one of, e.g., a tissue excision, a hemostatic treatment, an RF therapy, a thermal treatment, a cryogenic treatment, a brachytherapy treatment, a radiation therapy treatment, a therapeutic agent, a pharmaceutical agent, a genomic agent, and the like. The exemplary clamping forceps can include at least one of, e.g., a blade, a needle, an ablation instrument, a grinding mechanism, a suction mechanism, and the like, to support, in whole or in part, such treatment(s).

In some embodiments of the present disclosure, the clamping mechanism of the exemplary clamping forceps is actuated by at least one of e.g., a manual actuation, a motorized actuation, and the like. The clamping mechanism can include a sleeve configured and dimensioned to actuate the first clamp member and the second clamp member into a clamping orientation relative to a tumor or other structure based on a translation of the clamping mechanism relative to the elongated body section. The clamping mechanism can include at least one of, e.g., a cam-style mechanism, a spring-loaded mechanism, a gearing mechanism, a cable wire mechanism, a ratcheted mechanism, a motorized mechanism, a piezoelectric mechanism, a pneumatic mechanism, a solenoid actuator mechanism, a slide-crank mechanism, a slot yoke mechanism, a worm gear mechanism, a scissor mechanism, a rack and pinion mechanism, and the like.

In some embodiments of the present disclosure, the exemplary clamping forceps can be at least one of e.g., motorized, implemented manually by a surgeon, configured and dimensioned to be appended to a robotic arm, configured and dimensioned to be appended to a laparoscopic device, a portable laparoscopic device, an open-surgery device, a remote controlled device, an unpowered device, a powered device, an unpowered minimally-invasive device, configured and dimensioned to be laparoscopically introduced, configured and dimensioned to be introduced through a trocar, configured and dimensioned to be appended to an alternative unpowered, open-surgery, portable, powered, remote controlled, and/or laparoscopic instrument, appended to a powered minimally-invasive instrument and/or device, and the like. At least one of the head section, the elongated body section and the clamping mechanism can be fabricated from at least one of, e.g., a metallic material, a polymeric material, a composite material, and the like. The material of fabrication can further be, e.g., sterilizable, disposable, biodegradable, and the like.

In accordance with embodiments of the present disclosure, exemplary bioresorbable clamps are provided, generally including a first clamp member and a second clamp member configured and dimensioned to at least partially encircle a tumor or other structure. The exemplary bioresorbable clamps generally include a clamping mechanism for interlocking the first clamp member and the second clamp member relative to each other. Interlocking the first clamp member and the second clamp member relative to each other is generally effective to substantially restrict blood flow to the tumor or other structure. The clamping mechanism can generally be, e.g., a ratchet mechanism, a fixation mechanism, a release mechanism, and the like. The exemplary ratchet mechanism can generally be, e.g., at least one extension and at least one aperture configured and dimensioned to interlock relative to each other, and the like.

In accordance with embodiments of the present disclosure, exemplary methods of clamping with a bioresorbable clamp are also provided, generally including introducing the bioresorbable clamp to a surgical site. The exemplary bioresorbable clamp generally includes a first clamp member and a second clamp member configured and dimensioned to at least partially encircle a tumor or other structure. The exemplary bioresorbable clamp generally further includes a clamping mechanism for interlocking the first clamp member and the second clamp member relative to each other. The exemplary methods generally include positioning the first clamp member and the second clamp member so as to at least partially encircle the tumor or other structure. The exemplary methods generally further include clamping the first clamp member and the second clamp member around the tumor or other structure. Clamping the first clamp member and the second clamp member around the tumor or other structure is generally effective to substantially restrict blood flow to the tumor or other structure.

In accordance with aspects of the present disclosure, clamping forceps are provided that include first and second clamping members that are angularly oriented with respect to the operative handle section to facilitate surgeon viewing, ergonomics, and positioning of the clamping members relative to a desired anatomical location/region. The clamping members may define advantageous geometric configurations to facilitate positioning relative to an anatomical location/region. For example, angular joints or transitions may be provided such that the clamping members define advantageous geometric configurations, e.g., a substantially trapezoidal configuration, a compound curvature configuration or other angular configuration. The angular joints or transitions may be fixed, e.g., during clamping forceps fabrication, or variable at the time of surgery. The elongated body section that extends between the handle section and the clamping members may include a clamping mechanism and may define, in whole or in part, a substantially curved configuration to further enhance surgeon visibility, ergonomics, and positioning of the clamping members, e.g., when the surgical procedure is performed by way of a flank incision. Thus, a curved region in the transition from the handle section to the elongated body section may be advantageously incorporated into the clamping forceps design. The curved region may be fixed, i.e., established during fabrication of the clamping forceps, or variable such that the surgeon may select a desired curve for a specific procedure and then “fix” the selected curve for completion of the surgical procedure.

Mechanisms for detachment/reattachment of the clamping section relative to a subassembly defined by the elongated body section and the handle section may be provided to facilitate introduction of the clamping section to the desired clinical location and subsequent operative interaction therewith. Thus, in exemplary embodiments, a magnetic connection mechanism may be provided to facilitate intra-corporeal detachment/reattachment of the clamping section relative to the elongated body section/handle section subassembly. Gearing mechanisms, e.g., worm gear mechanisms, may be associated with the clamping members, e.g., an end effector subassembly that includes the clamping members, to facilitate approximation of the clamping members through extra-corporeal operative control exercised by the surgeon. The connection mechanism may support rotational functionality such that the clamping members may be reoriented relative to the desired clinical location/region, e.g., to encircle a tumor or the like.

In an exemplary clinical implementation of disclosed clamping forceps according to the present disclosure, a surgeon may introduce the clamping members to a surgical region, e.g., through an incision originally created for placement of a trocar port/cannula. Thereafter, the surgeon may reinsert the trocar port/cannula and connect the clamping members to an elongated body section/handle section subassembly that is introduced through the reinserted trocar port/cannula. Intra-corporeal connection of the clamping members relative to the elongated body section/handle section subassembly is accomplished through a mating mechanism, e.g., a magnetic mechanism that operatively connects the clamping members relative to the elongated body section/handle section subassembly. Thereafter, clamping action of the clamping members relative to a desired surgical location/region, such as a tumor, may be achieved via extra-corporeal control through the re-inserted trocar port/cannula. Once the clamping members have been clamped in a desired fashion, the clamping members may be detached from the elongated body section/handle section subassembly, thereby permitting the clamping members to be left in place within the body cavity while the elongated body section/handle section subassembly is removed from the trocar port/cannula, thereby freeing up the trocar port/cannula for introduction of other surgical devices. At the conclusion of the surgical procedure, the clamping members may be removed from the surgical region independent of the trocar port/cannula, e.g., in conjunction with excised tissue.

Other objects and features will become apparent from the following detailed description considered in conjunction with the accompanying drawings. It is to be understood, however, that the drawings are designed as an illustration only and not as a definition of the limits of the invention.

### BRIEF DESCRIPTION OF THE DRAWINGS

To assist those of skill in the art in making and using the disclosed devices and associated methods, reference is made to the accompanying figures, wherein:

FIGS. 1A-G show perspective, side and detailed views of exemplary embodiments of a clamping forceps;

FIGS. 2A-C show a stress simulation analysis and displacement analysis for exemplary first and second clamp members of exemplary embodiments of a clamping forceps;

FIGS. 3A and B show a perspective view and cross-sectional view of an exemplary embodiment of a motorized clamping forceps;

FIGS. 4A and B show an exemplary drive motor of an exemplary embodiment of a clamping forceps;

FIGS. 5A-C show an exemplary sensor implemented in conjunction with an exemplary embodiment of a clamping forceps and exemplary blood-flow detection diagrams;

FIGS. 6A and B show exemplary sensors implemented in conjunction with an exemplary embodiment of a clamping forceps;

FIG. 7 shows a CT scan of a cross-section of a patient with a tumor;

FIG. 8 shows an additional tumor contour image obtained by a sensor of an exemplary embodiment of a clamping forceps;

FIG. 9 shows a detailed view of tissue characteristics of an organ obtained by a sensor of an exemplary embodiment of a clamping forceps;

FIG. 10 shows an exemplary control diagram for an exemplary sensor control system;

FIGS. 11A-C show an exemplary visual feedback user interface;

FIG. 12 shows an exemplary visual feedback user interface;

FIG. 13 shows an alternative control diagram for an exemplary sensor control system;

FIGS. 14A-C show an exemplary embodiment of a clamping forceps, an exemplary control diagram and an exemplary output waveform;

FIG. 15 shows an alternative control diagram for an exemplary sensor control system;

FIGS. 16A and B show an exemplary single-pole solenoid actuation mechanism and a flow chart for an exemplary single-pole solenoid actuation mechanism;

FIGS. 17A and B show electrical circuit diagrams for an exemplary single-pole solenoid actuation mechanism;

FIG. 18 shows an exemplary double-pole solenoid actuation mechanism;

FIG. 19 shows a flow chart for an exemplary double-pole solenoid actuation mechanism;

FIGS. 20A and B show electrical circuit diagrams for an exemplary double-pole solenoid actuation mechanism;

FIGS. 21A and B show an exemplary variable position solenoid actuation mechanism and a flow chart for an exemplary variable position solenoid actuation mechanism;

FIGS. 22A-C show an exemplary embodiment of a transient ischemia clamping forceps;

FIGS. 23A-C show an alternative exemplary embodiment of a transient ischemia clamping forceps;

FIGS. 24A-C show an alternative exemplary embodiment of a transient ischemia clamping forceps;

FIGS. 25A-C show an alternative exemplary embodiment of a transient ischemia clamping forceps;

FIG. 26 shows a toggle switch of an exemplary embodiment of a clamping forceps;

FIGS. 27A-D show an exemplary embodiment of a suction mechanism of a clamping forceps and implementation of surgical suture clips;

FIGS. 28A-C show an exemplary clamping forceps for implementation with surgical suture clips;

FIGS. 29A-C show an exemplary clamping forceps for implementation with surgical suture clips;

FIGS. 30A-D show an exemplary embodiment of a clamping forceps for manual laparoscopic introduction;

FIG. 31 shows an exemplary embodiment of a clamping forceps for manual open surgery operation;

FIGS. 32A-C show an exemplary embodiment of a clamping forceps for manual open surgery operation;

FIGS. 33A-C show an exemplary embodiment of a clamping forceps for manual open surgery operation;

FIGS. 34A-D show an exemplary embodiment of a clamping forceps with malleable first and second clamp members;

FIGS. 35A-F show exemplary embodiments of malleable first and second clamp members;

FIGS. 36A-D show an exemplary embodiment of self-opening first and second clamp members;

FIGS. 37A and B show an exemplary embodiment of a bioresorbable clamp;

FIGS. 38A and B show an exemplary embodiment of a bioresorbable clamp; and

FIGS. 39A-C show an exemplary embodiment of a bioresorbable clamp;

FIGS. 40-42 show an exemplary embodiment of a clamping forceps that include curved/angular features that facilitate viewing and positioning of clamping members in a desired clinical location;

FIGS. 43 and 44 illustrate positioning of the clamping forceps of FIGS. 40-42 relative to a desired clinical location;

FIG. 45 is a flowchart showing steps relative to an exemplary clinical procedure using certain clamping forceps of the present disclosure; and

FIGS. 46-47 show an alternative end effector and exemplary mating mechanisms that facilitate cooperation between a clamping section and an elongated body section/handle section subassembly according to the present disclosure.

### DESCRIPTION OF EXEMPLARY EMBODIMENTS

In accordance with embodiments of the present disclosure, exemplary clamping forceps and associated methods are provided. Although the exemplary embodiments discussed herein include a plurality of varying components and/or features, those of ordinary skill in the art should understand that the plurality of varying components and/or features may be interchanged between the exemplary clamping forceps. For example, in some exemplary embodiments, the clamping forceps may include, e.g., one, two, three, four, five, and the like, of the plurality of components and/or features. In other exemplary embodiments, the clamping forceps may include all of the plurality of components and/or features discussed herein.

The exemplary embodiments of clamping forceps discussed herein generally alleviate the concerns of WIT by localizing ischemia to the tissue to be excised. For example, the clamping forceps may be placed around a tumor on an organ, e.g., a kidney, liver, and the like, and the accompanying negative surgical margin. By effectively managing the clamping pressure presented by the clamping forceps on the organ, a surgeon is generally able to regulate and/or restrict blood flow to the tumor, thereby localizing ischemia to the tumor and isolating ischemia from the remaining body of the organ. Thus, a surgeon generally is not required to regulate and/or restrict blood flow to the entire organ via a clamp on, for example, a renal artery and vein in order to perform the operation. Further, the surgeon is generally no longer under the time pressure to complete a surgical procedure within the, e.g., WIT time frame and is able to take the necessary time needed to properly establish hemostasis, excise the tumor and negative surgical margin, and close the void in the parenchyma that resulted from excision of the tumor.

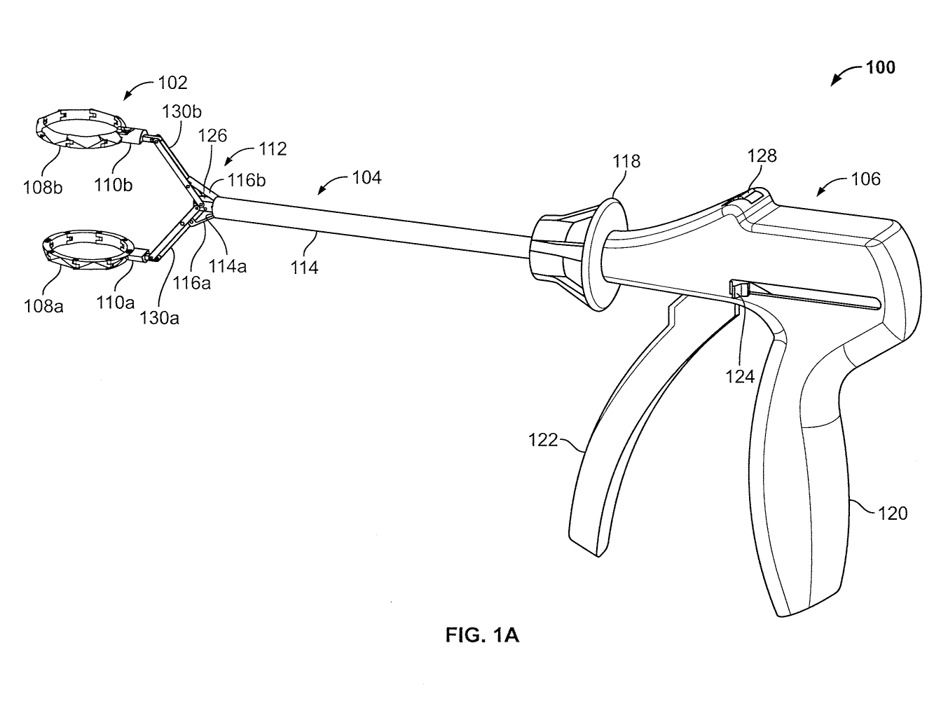

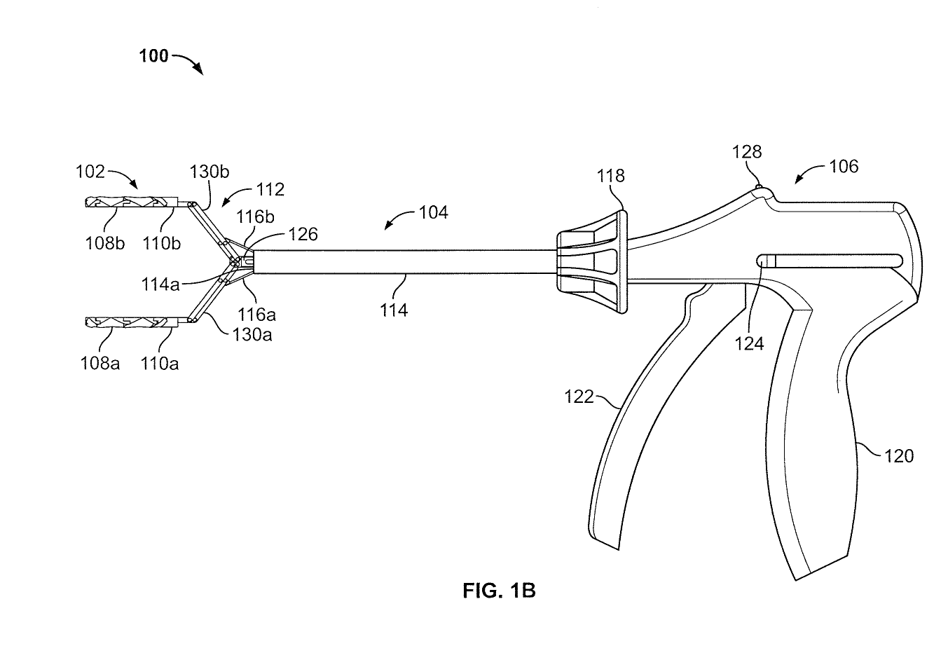

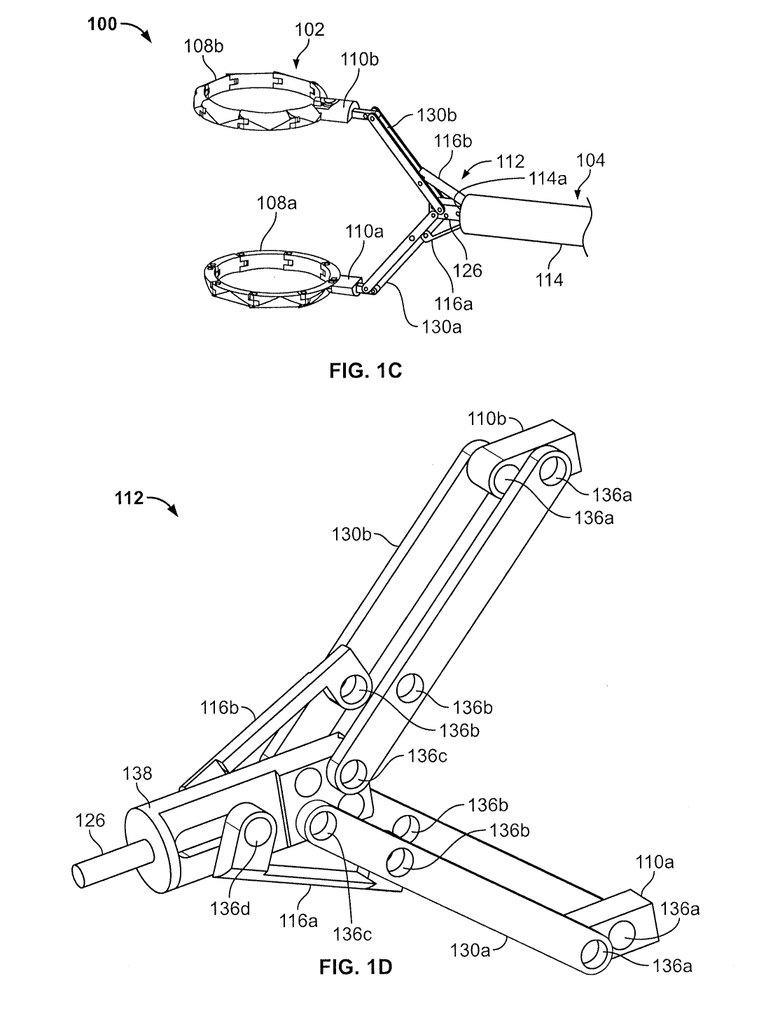

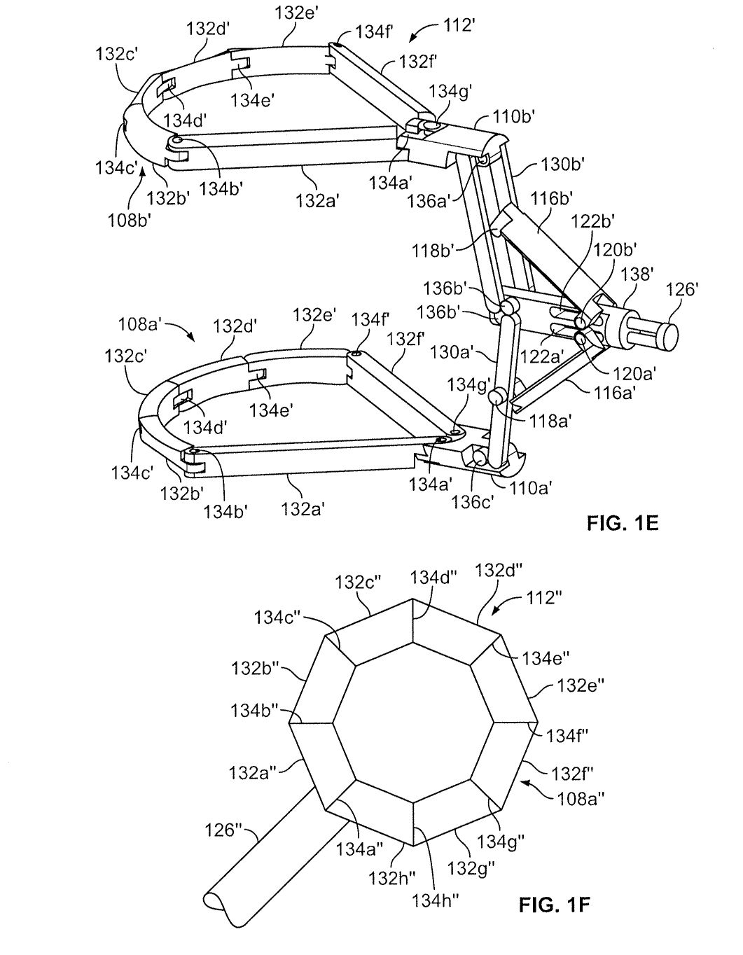

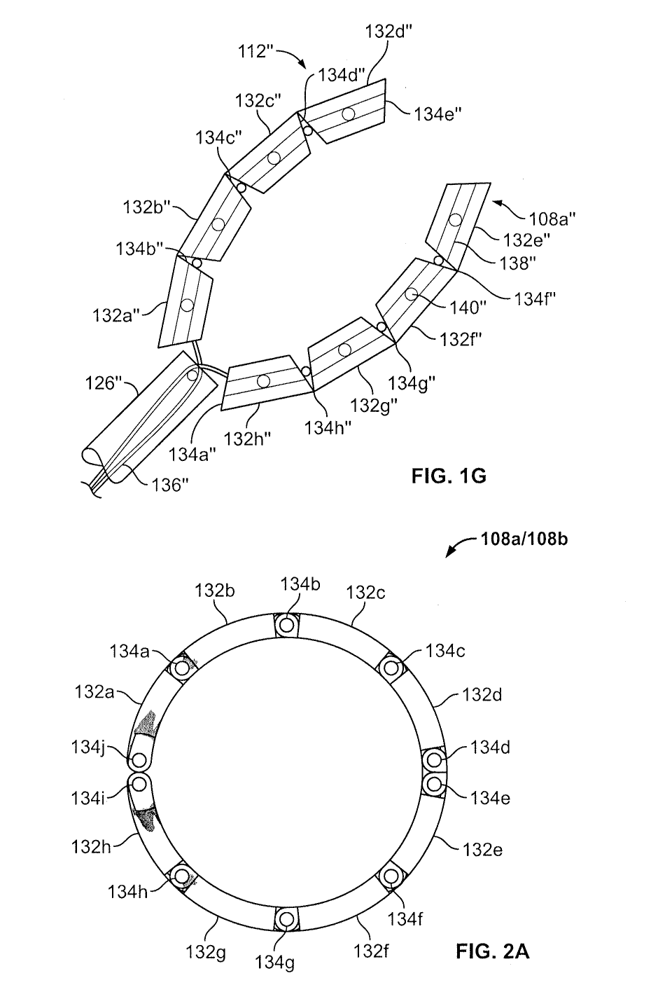

Turning now to FIGS. 1A-E, exemplary clamping forceps 100 are illustrated. In particular, the clamping forceps 100 are illustrated as a manually-operated, laparoscopic-surgery and/or minimally invasive surgery device. It should be understood that in other embodiments, the exemplary clamping forceps 100 can be, e.g., motorized device, an open-surgery device, a robotic device, and the like. The clamping forceps 100 generally includes a head section 102, an elongated body section 104, and a handle section 106. The head section 102 generally includes a first clamp member 108a and a second clamp member 108b. The first and second clamp members 108a and 108b can be fabricated as, e.g., a solid clamp member body, a plurality of interconnected linkages, and the like. As can be seen in FIG. 1A, the exemplary first and second clamp members 108a and 108b are defined by a plurality of interconnected linkages configured and dimensioned to fold so as to fit through a trocar port, e.g., an approximately 5 mm diameter trocar, 8 mm diameter trocar, 10 mm diameter trocar, 12 mm diameter trocar, 15 mm diameter trocar, and the like. For example, the first and second clamp members 108a and 108b may be administered to the target area via, e.g., an open incision, laparoscopically through trocar port, a remote control, a wireless control, an automated manner, a robotically assisted manner, a natural orifice, and the like. In other exemplary embodiments, the first and second clamp members 108a and 108b can be defined by, e.g., a circular, an ovular, a polygonal, a variable, a C-shaped, a J-shaped, an L-shaped configuration, a malleable, a topographically contoured and the like, configuration. In some exemplary embodiments, the first and second clamp members 108a and 108b may be substantially, e.g., 15 mm, 30 mm, 45 mm, 60 mm, and the like, in diameter.

As would be understood by those of ordinary skill in the art, the first and second clamp members 108a and 108b can generally be positioned on and/or around so as to encompass at least a section of an organ, e.g., a kidney, a liver, or other structure. Thus, for example, a tumor may be positioned within the inner perimeter of the first and second clamp members 108a and 108b, i.e., the surgical site. Thus, a user can clamp the exemplary clamping forceps 100 around a tumor such that a user can perform a surgical operation, e.g., excise the tumor, by operating within the perimeter of the first and second clamp members 108a and 108b. The clamping of the first and second clamp members 108a and 108b regulates and/or prevents the blood flow passing to the surgical site within the perimeter of the first and second clamp members 108a and 108b, while permitting substantially regular blood flow to pass to the rest of the organ, e.g., the kidney, liver, and the like. The substantially regular blood flow to the rest of the organ generally reduces the concern of WIT during surgical procedures, thereby typically increasing the time a surgeon can operate on an organ.

The first and second clamp members 108a and 108b can be in detachable cooperation with respect to the head section 102. In particular, the first and second clamp members 108a and 108b can be detachably secured to a clamping mechanism 112 at the first and second clamp connectors 110a and 110b, respectively. Thus, a surgeon can interchange a plurality of configurations and/or dimensions of first and second clamp members 108a and 108b based on, e.g., the diameter, profile, and the like, of the target tissue (e.g., target tumor). The interchangeable and/or detachable first and second clamp members 108a and 108b and/or entire head section 102 also permit customization of the exemplary clamping forceps 100 for adaptability to a specific surgical environment and/or tumor. In some exemplary embodiments, the handle section 106 may be detachably secured in mechanical communication to the elongated body section 104. The exemplary clamping forceps 100 generally include an actuator (not shown) for releasing the first and second clamp members 108a and 108b from the head section 102 at the first and second clamp connectors 110a and 110b. As would be understood by those of ordinary skill in the art, a replacement pair of first and second clamp members 108a and 108b may be, e.g., snapped, screwed, locked, and the like, into the first and second clamp connectors 110a and 110b.

The clamping mechanism 112 can be configured and dimensioned such that distal movement of the clamping mechanism 112 relative to the elongated body section 104 functions to move the first clamp member 108a and the second clamp member 108b into a clamping orientation relative to a tumor or other structure. Further, the first and second clamp members 108a and 108b may be spring-loaded relative to each other. As illustrated in the exemplary embodiment of FIGS. 1A and 1D, the clamping mechanism 112 can be configured as, e.g., a cam-style mechanism, and includes a shaft 126 passing through the sleeve 114, e.g., a tube, and the like, of the elongated body section 104. Further, the clamping mechanism 112 and/or the shaft 126 are in mechanical communication with the handle section 106. The clamping mechanism 112 can generally be movably mounted with respect to the elongated body section 104. Thus, as would be understood by those of ordinary skill in the art, as the clamping mechanism 112 is mechanically actuated by the handle section 106, the clamping mechanism 112 can be translated in and/or out of the sleeve 114.

In particular, the exemplary clamping forceps 100 can be introduced into a surgical site through a trocar port (i.e., cannula) by initially folding the first and second clamp members 108a and 108b by actuating the clamping mechanism 112 to be translated into the sleeve 114. The clamping mechanism 112 can be further actuated to pull the folded first and second clamp members 108a and 108b into the sleeve such that the sleeve 114 can then be introduced into the surgical site through the trocar port. Once the clamping forceps 100 have been introduced into the surgical site, the clamping mechanism 112 can be actuated to push the folded first and second clamp members 108a and 108b out of the sleeve 114. In some exemplary embodiments, the first and second clamp members 108a and 108b may be spring-loaded such that extraction or exposure of the first and second clamp members 108a and 108b out of or from the sleeve 114 automatically expands the first and second clamp members 108a and 108b into a predetermined configuration. The spring-loaded first and second clamp members 108a and 108b can further extend relative to each other as permitted by the positioning of the sleeve 114 relative to the first and second actuation members 116a and 116b. As would be understood by those of ordinary skill in the art, as the clamping mechanism 112 is translated in and/or out of the sleeve 114, a distal sleeve edge 114a of the sleeve 114 actuates the first and second actuation members 116a and 116b such that the clamping mechanism 112 moves the first and second clamp members 108a and 108b closer and/or farther relative to each other in a clamping orientation. In particular, actuation of the first and second actuation members 116a and 116b in turn actuates the first and second connection members 130a and 130b. The first and second connection members 130a and 130b may be movably connected to the shaft 126 and the first and second actuation members 116a and 116b. Further, the first and second connection members 130a and 130b may be rigidly connected to the first and second clamp connectors 110a and 110b.

The elongated body section 104 is generally connected to the handle section 106 by an articulation joint 118. The articulation joint 118 generally provides the ability to rotate the handle section 106 and/or the first and second clamp members 108a and 108b independently of each other at substantially 360° along the axis of the shaft 126, while still enabling the surgeon to apply the compressive and/or clamping forces necessary to conduct the surgical procedure. The surgeon is thereby generally provided the flexibility to position and/or reposition the exemplary clamping forceps 100 as necessary generally without fear of releasing the clamping pressure on the organ. In some exemplary embodiments, a break in the shaft 126 can exist at the articulation joint 118, i.e., an articulation lock, and the distal and proximal sections of the shaft 126 are generally connected via a coupling (not shown). As would be understood by those of ordinary skill in the art, rotation of the articulation joint 118, e.g., counter-clockwise, generally loosens the pressure of the coupling on the shaft 126. Similarly, rotation of the articulation joint 118, e.g., clockwise, generally tightens the pressure of the coupling on the shaft 126. In some exemplary embodiments, a cam lock (not shown) may be utilized to fixate the rotation of the articulation joint 118 completely. The handle section 106 generally includes a grip 120 and a trigger 122 for actuating the clamping mechanism 112. The grip 120 may be formed, e.g., ergonomically, such that the user, i.e., the surgeon, can comfortably and securely grasp the grip 120 during implementation of the clamping forceps 100. The grip 120 generally includes at least one of e.g., a smooth, a textured, and the like, surface to prevent slippage of the user's hand during operation of the clamping forceps 100.

The clamping mechanism 112 can be actuated at variable rates of clamping action, e.g., a fine adjustment, a gross adjustment, and the like. The gross adjustment of clamping action can be, e.g., substantially one centimeter increments, and the like. The fine adjustment of clamping action can be, e.g., substantially one millimeter increments, substantially one hundred micrometer increments, and the like. The exemplary clamping forceps 100 generally include a trigger 122, i.e., a fine adjustment regulator, and a gross adjustment regulator 124 for regulating the variable rates of clamping action and/or pressure. In some exemplary embodiments, the fine and gross adjustment may be regulated by one component, e.g., one trigger 122. The gross adjustment regulator 124 can be, e.g., spring-loaded, and in mechanical communication with the shaft 126 of the clamping mechanism 112. As would be understood by those of ordinary skill in the art, the gross adjustment regulator 124 can be actuated, e.g., pulled, by a surgeon, thereby actuating, e.g., pulling, the shaft 126 of the clamping mechanism 112 through the sleeve 114. Pulling the shaft 126 into the sleeve 112 further actuates the first and second clamp members 108a and 108b to move relative to each other in a clamping orientation. Thus, the gross adjustment regulator 124 may be implemented by a user to regulate the positioning of the first and second clamp members 108a and 108b relative to a tumor, organ and/or other structural surface by larger distance increments, e.g., substantially 1 cm increments, 2 cm increments, 3 cm increments, 4 cm increments, 5 cm increments, and the like, than the fine adjustment regulator.

Similarly, the trigger 122, i.e., the fine adjustment regulator, can be in mechanical communication with the shaft 126 of the clamping mechanism 112. Thus, as a user pulls, e.g., repeatedly compresses, and the like, the trigger 122, the first and second clamp members 108a and 108b can be actuated to move relative to each other in a clamping orientation by small distance increments, e.g., one millimeter increments, one hundred micrometer increments, and the like. In some exemplary embodiments, the progressive transition between the gross and fine adjustment regulators may be, e.g., continuous, automatic, manual, and the like, to ensure a desired clamping pressure is provided against the organ. For example, FIG. 1D illustrates a clamping mechanism 112 which provides for gross and/or fine adjustment regulation of a substantially uniform clamping pressure. The variable rates of clamping action allow a user to initially position the first and second clamp members 108a and 108b around a tumor, organ or other structure in a desired location and to regulate the clamping pressure on the tumor, organ or other structure by actuating the clamping forceps 100 with the fine adjustment regulator. This permits the user to achieve a desired clamping pressure therearound, e.g., a sufficient clamping pressure to regulate blood flow passing to a tumor as desired without crushing and/or damaging the organ and/or healthy tissue. In some exemplary embodiments, a direction switch (not shown) may be implemented to reverse the direction of clamping action. Once a user has achieved the desired clamping pressure around the tumor, organ or other structure, a clamping position lock 128 may be implemented to lock the clamping mechanism 112 in the desired position. Thus, the clamping position lock 128 ensures that the first and second clamp members 108a and 108b remain positioned in the desired clamping orientation and/or position relative to each other. Once the surgical procedure has been completed, the clamping position lock 128 may be released, e.g., a quick release may be actuated, to permit the first and second clamp members 108a and 108b to be actuated farther apart relative to each other in the clamping orientation, thus releasing the organ or other structure being operated upon.

Turning now to FIG. 1B, a side view of an exemplary clamping forceps 100 is provided. As can be seen, the first and second clamp members 108a and 108b can be oriented relative to each other in a clamping orientation such that when the clamping mechanism 112 is actuated, the first and second clamp members 108a and 108b move relative to each other while maintaining substantially parallel clamping surfaces, i.e. the surface of the first and second clamp members 108a and 108b which is positioned against the tumor, organ or other structure to be clamped. In some exemplary embodiments, the clamping surfaces of the first and second clamp members 108a and 108b can be, e.g., curved, straight (parallel), angled, variable, and the like, relative to each other. The exemplary clamping surfaces of the first and second clamp members 108a and 108b can be fabricated from, e.g., rigid material, soft material, a combination of various material compositions, and the like, to reduce tissue trauma. The soft material of fabrication can be, e.g., a gel, and the like.

In some exemplary embodiments, the plurality of interconnected linkages of the first and second clamp members 108a and 108b may rigidly maintain the topography of the clamping surface, e.g., curved, straight (parallel), angled, and the like, when the first and second clamp members 108a and 108b have been unfolded into their predetermined configuration. In other exemplary embodiments, the plurality of interconnected linkages of the first and second clamp members 108a and 108b may function to provide a topographically variable clamping surface so as to conform to a topography of tissue surrounding a tumor and/or tumor topography. For example, the topography of the clamping surface of the first and second clamp members 108a and 108b may conform to at least one of the topography of the tissue surrounding a tumor and the tumor itself as the first and second clamp members 108a and 108b are clamped around the tumor. In other exemplary embodiments, the topography of the clamping surface of the first and second clamp members 108a and 108b may be regulated by a user at a user interface, e.g., the handle section 106. Thus, rather than having a rigid and/or uniform topography, the topographically variable clamping surfaces of the first and second clamp members 108a and 108b generally permits the exemplary clamping forceps to adapt to the topography of the surgical site in order to ensure a stronger, more accurate, and/or uniform clamping action/force distribution around the tumor to be excised.

With reference to FIG. 1C, a detailed view of the head section 102 of an exemplary clamping forceps 100 is illustrated. In particular, the first and second connection members 130a and 130b can be movably connected to the first and second clamp connectors 110a and 110b, the shaft 126, and the first and second actuation members 116a and 116b at, e.g., hinge-type joints, and the like. Thus, as the shaft 126 is pulled into the sleeve 114, first and second actuation members 116a and 116b are compressed by the distal sleeve edge 114a, which in turn compresses the first and second connection members 130a and 130b. The compression of the first and second connection members 130a and 130b further moves the first and second clamp members 108a and 108b close to each other in a clamping orientation. As discussed above, actuation of the clamping mechanism 112, i.e., translation of the shaft 126 in and out of the sleeve 114, allows a user to regulate the clamping pressure applied by the clamping surfaces of the first and second clamp members 108a and 108b against a tumor, organ or other structure. In some exemplary embodiments, rather than implementing a sleeve 114, the clamping mechanism 112 may be actuated by mechanical actuation of the first and second actuation members 116a and 116b through mechanical communication with the shaft 126 and/or the handle section 106 and/or the clamping mechanism 112 may be, e.g., a cam-style mechanism, a spring-loaded mechanism, a gearing mechanism, a cable wire mechanism, a ratcheted mechanism, a motorized mechanism, a piezoelectric mechanism, a pneumatic mechanism, a solenoid actuator mechanism, a slide-crank mechanism, a slot yoke mechanism, a worm gear mechanism, a scissor mechanism, a rack and pinion mechanism, and the like.

FIG. 1D illustrates an exemplary clamping mechanism 112, generally including first and second actuation member 116a and 116b, first and second clamp connectors 110a and 110b, first and second connection members 130a and 130b, a shaft 126, and interconnecting member 138. Although not illustrated in FIG. 1D, it should be understood that the components of the clamping mechanism 112 are generally movably interconnected at joints 136a-d with, e.g., pins, bearings, and the like. In particular, the first and second connection members 130a and 130b are generally movably connected to the first and second clamp connectors 110a and 110b, respectively, at joints 136a, and movably connected to the interconnecting member 138 at joints 136c. The first and second actuation members 116a and 116b are generally movably connected to the first and second connection members 130a and 130b at joints 136b, and movably connected to the shaft 126 and the interconnecting member 138 at joints 136d. As discussed above, actuation of the shaft 126, i.e., translation of the shaft 126 in and/or out of the sleeve 114, mechanically actuates the first and second clamp members 108a and 108b to move relative to each other in a clamping orientation. Although illustrated as a cam-style clamping mechanism 112, in other exemplary embodiments, the clamping mechanism 112 can be, e.g., a spring-loaded mechanism, a gearing mechanism, a cable wire mechanism, a ratcheted mechanism, a motorized mechanism, a piezoelectric mechanism, a pneumatic mechanism, a solenoid actuator mechanism, a slide-crank mechanism, a slot yoke mechanism, a worm gear mechanism, a scissor mechanism, a rack and pinion mechanism, and the like.

FIG. 1F illustrates an exemplary clamping mechanism 112′, generally including first and second actuation members 116a′ and 116b′, first and second clamp connectors 110a′ and 110b, first and second connection members 130a′ and 130b′, a shaft 126′, and interconnecting member 138′. Although not illustrated in FIG. 1E, it should be understood that the components of the clamping mechanism 112′ are generally movably interconnected at joints 136a′-c′ with, e.g., pins, bearings, and the like. In particular, the first and second connection members 130a′ and 130b′ are generally movably connected to the first and second clamp connectors 110a′ and 110b′, respectively, at joints 136a′ and 136c′, and movably connected to the interconnecting member 138′ and joints 136b′. The first and second actuation members 116a′ and 116b′ are generally movably and/or slidably connected to the first and second connection members 130a′ and 130b′ at joints 118a′ and 118b′, and movably and/or slidably connected to the shaft 126′ and the interconnecting member 138′ at joints 120a′ and 120b′. In particular, the joints 120a′ and 120b′ generally slide in tracks 122a′ and 122b′ to actuate the first and second actuation members 116a′ and 116b′. As discussed above, actuation of the shaft 126′, i.e., translation of the shaft 126′ in and/or out of the sleeve 114, mechanically actuates the first and second clamp members 108a′ and 108b′ to move relative to each other in a clamped orientation. Although illustrated as a dual-track clamping mechanism 112′, in other exemplary embodiments, the clamping mechanism 112′ can be, e.g., a cam-style mechanism, a spring-loaded mechanism, a motorized mechanism, a piezoelectric mechanism, a pneumatic mechanism, a solenoid actuator mechanism, a slide-crank mechanism, a slot yoke mechanism, a worm gear mechanism, a scissor mechanism, a rack and pinion mechanism, and the like.

The exemplary first and second clamp members 108a′ and 108b′ of FIG. 1E can define, e.g., a substantially C-shaped configuration, J-shaped configuration, an L-shaped configuration, variable configuration, and the like. In particular, the first and second clamp members 108a′ and 108b′ generally define a plurality of linkages 132a′-f, e.g., chain-link components, movably interconnected relative to each other at joints 134a′-g′, e.g., hinge-type joints, and the like. Although illustrated with six linkages 132a′-f, in some exemplary embodiments, the first and second clamp members 108a′ and 108b′ can include, e.g, two, three, four, five, six, seven, eight, nine, ten, and the like, linkages.

Turning now to FIGS. 1F and G, an exemplary cable drive clamping mechanism 112″ is illustrated. Although illustrating only a first clamp member 108a″, it should be understood that the second clamp member (not shown) is substantially similar in function and/or structure to the first clamp member 108a″. The first clamp member 108a″ generally defines a plurality of linkages 132a″-h″, e.g., chain-link components, movably interconnected relative to each other at joints 134a″-h″, e.g., hinge-type joints, springs, and the like. Although illustrated with eight linkages 132a″-h″, in some exemplary embodiments, the first clamp member 108a″ can include, e.g., two, three, four, five, six, seven, eight, nine, ten, and the like, linkages. The plurality of linkages 132a″-h″ generally define an interior passage, e.g., a hollow interior, configured and dimensioned as a plurality of internal rails 138″. The plurality of linkages 132a″-h″ generally further include a plurality of guide pins 140″ configured and dimensioned to slide within the plurality of respective internal rails 138″. At least one cable 136″ can generally pass through a hollow interior of the shaft 126″ and mechanically connect to a user interface. As would be understood by those of ordinary skill in the art, actuating the clamping mechanism 112″ to tighten and/or loosen the cable 136″ generally actuates the plurality of guide pins 140″ to translate within the internal rails 138″ such that the configuration of the first clamp member 108a″ can be varied. In some exemplary embodiments, the configuration of the first clamp member 108a″ can be actuated to be, e.g., a C-shaped configuration, a J-shaped configuration, a circular configuration, an oval configuration, and the like. In some exemplary embodiments, loosening the cable 136″ generally permits the spring hinges at joints 134a″-h″ to actuate, thereby expanding the first clamp member 108a″ from a substantially folded to a substantially open configuration after introduction through a trocar.

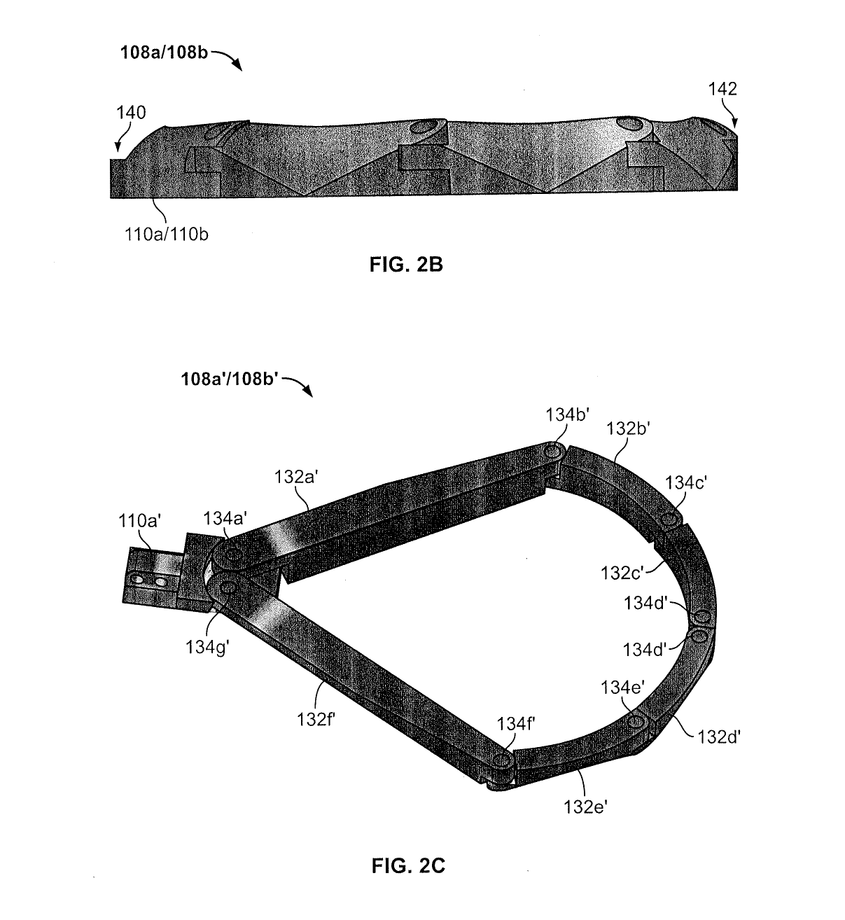

Turning to FIG. 2A, a stress simulation analysis is illustrated for the first and second clamp members 108a and 108b. Although one clamp member is illustrated in FIG. 2A, it should be understood that the illustration represents the simulated stress levels in each of the first and second clamp members 108a and 108b. Further, the stress simulation is provided for first and second clamp members 108a and 108b which are defined by a plurality of linkages 132a-132h interconnected by a plurality of joints 134a-134j, e.g., hinge-type joints. As can be seen from FIG. 2A, only minimal stress levels were generally found at linkages 132a and 132h and joints 134a, 134h, 134i and 134j. Although illustrated as eight linkages 132a-h, in some exemplary embodiments, the first and second clamp members 108a and 108b can include, e.g, two, three, four, five, six, seven, eight, nine, ten, and the like, linkages. In some exemplary embodiments, different sizes and/or configurations of linkages may be implemented for varying the perimeter extent of the first and second clamp members 108a and 108b. It should also be understood by those of ordinary skill in the art that the finite element analysis (FEA) illustrated in FIG. 2A is merely exemplary and may differ for alternative configurations of first and second clamp members 108a and 108b based on, e.g., materials of fabrication, positioning at different areas on an organ, the implementation of less and/or more linkages 132a-h, and the like.

With reference to FIG. 2B, an exemplary displacement analysis is illustrated for the first and second clamp members 108a and 108b. In particular, FIG. 2B illustrates the maximum displacement of the first and second clamp members 108a and 108b when a clamping pressure is applied against, e.g., an organ, a tumor, and the like. As can be seen from FIG. 2B, while the proximal end 140, i.e., the end defined by first and second clamp connectors 110a and 110b, generally does not exhibit displacement during application of a clamping pressure, the distal end 142 generally exhibits the point of maximum displacement of the first and second clamp members 108a and 108b.

With reference to FIG. 2C, a stress simulation analysis is illustrated for the first and second clamp members 108a′ and 108b′. Although one clamp member is illustrated in FIG. 2B, it should be understood that the illustration represents the simulated stress levels in both the first and second clamp members 108a′ and 108b′ which are defined by a plurality of linkages 132a′-f interconnected by a plurality of joints 134a′-g′, e.g., hinge-type joints, and the like. As can be seen from FIG. 2C, only minimal stress levels were generally found near linkages 134a′, 134b′, 134f and 134g′. It should also be understood by those of ordinary skill in the art that the finite element analysis (FEA) illustrated in FIG. 2C is merely exemplary and may differ for alternative configurations of first and second clamp members 108a′ and 108b′ based on, e.g., materials of fabrication, positioning at different areas on an organ, the implementation of less and/or more linkages 132a′-f, and the like.

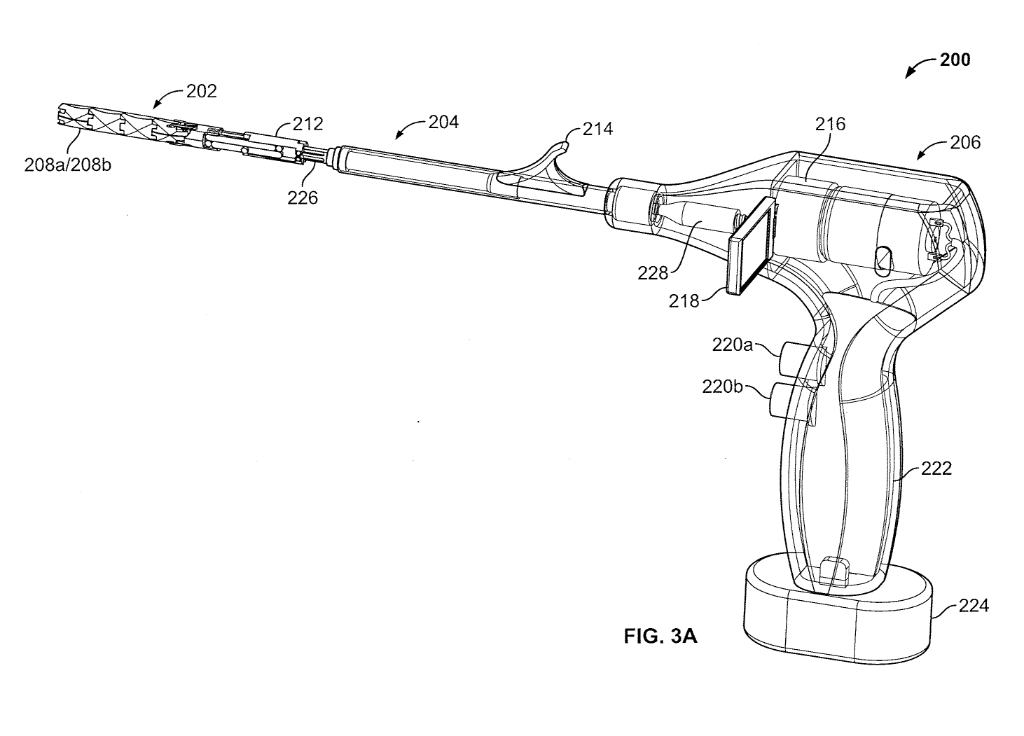

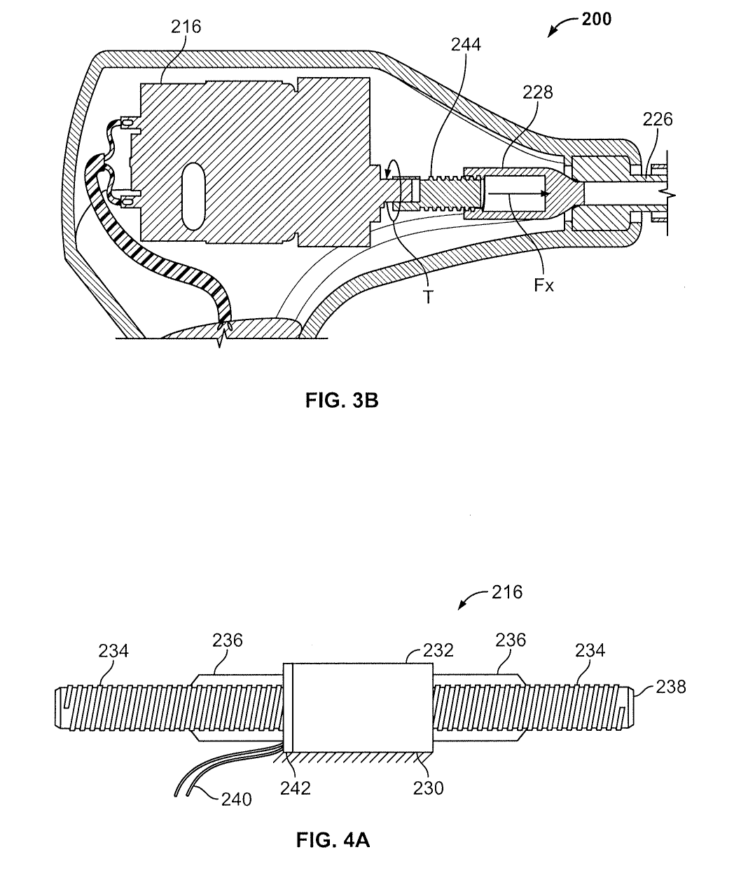

Turning now to FIG. 3A, an exemplary embodiment of motorized clamping forceps 200 for laparoscopic surgery is provided. The exemplary clamping forceps 200 is substantially similar in functionality and structure as the clamping forceps 100 discussed above, except for the features discussed herein. In particular, the exemplary clamping forceps 200 generally includes a head section 202, an elongated body section 204 and a handle section 206. In some exemplary embodiments, the head section 102 may be implemented in conjunction with the exemplary clamping forceps 200. Similarly, in some exemplary embodiments, the head section 202 may be implemented in conjunction with the exemplary clamping forceps 100, unless electronics require the use of the motorized clamping forceps 200. As illustrated in FIG. 3A, the first and second clamp members 208a and 208b have been folded to fit within a trocar port, e.g., a substantially 5 mm, 8 mm, 10 mm, 12 mm, 15 mm, and the like, trocar port/cannula. A clamping mechanism 212 is generally movably mounted with respect to the elongated body section 204. The clamping mechanism 212 of FIG. 3A is illustrated in a substantially folded configuration in order to fit within a trocar port/cannula. The clamping mechanism 212 generally includes a shaft 226 mechanically connected to the elongated body section 204 and/or the handle section 206. The elongated body section 204 further generally includes a shaft extension 214, i.e., a shaft extension and articulation. The shaft extension 214 can be implemented in an exemplary clamping mechanism 112 as discussed above, e.g., a cam-style mechanism, a spring-loaded mechanism, a motorized mechanism, a piezoelectric mechanism, a pneumatic mechanism, a solenoid actuator mechanism, a slide-crank mechanism, a slot yoke mechanism, a worm gear mechanism, a scissor mechanism, a rack and pinion mechanism, and the like. In particular, the shaft 126 can be actuated, e.g., in and/or out, relative to the interconnecting member 138, thereby pushing on first and second actuation arms 116a and 116b and first and second connection members 130a and 130b, as illustrated in FIG. 1D.

The handle section 206 of the exemplary clamping forceps 200 generally includes a drive motor 216 with a clamping force regulator 228, i.e., a clamping force governor. For example, the drive motor 216 may be a linear motor actuator with a plurality of differently sized tracks and/or threads (fine/coarse threads) to permit variable clamping action of the first and second clamp members. (See, e.g., RB-30GM DC Carbon-Brush Motor, ISL Products, Inc. (2012)). As can be seen from the cross-sectional view of the handle section 206 in FIG. 3B, an interconnecting member 244, e.g., a threaded shaft, and the like, generally mechanically connects the drive motor 216 and the clamping force regulator 228. The interconnecting member 244 generally converts the rotational torque transmission of the drive motor 216 into a linear force, e.g., between about 0 N and about 555 N, which translates the shaft such that the first and second clamp members 208a and 208b are actuated accordingly. The drive motor 216 may further be electrically connected to a grip 222 which includes a gross adjustment trigger 220a and a fine adjustment trigger 220b. As discussed above, the gross adjustment trigger 220a permits a user to regulate the clamping action at large increments, e.g., one centimeter increments, and the like. The fine adjustment trigger 220b permits a user to regulate the clamping action at small increments, e.g., one millimeter increments, one hundred micrometer increments, and the like. For example, gross and fine adjustment via the gross adjustment trigger 220a and the fine adjustment trigger 220b may be achieved through separate gearing. In some exemplary embodiments, a trigger 220a may be implemented to regulate the clamping action to clamp the first and second clamp members 208a and 208b relative to each other for both the fine and gross adjustment, and trigger 220b may be implemented to regulate the clamping action to unclamp the first and second clamp members 208a and 208b.

The handle section 206 generally further includes a real-time display 218, e.g., a fold-out display, which provides visual feedback with respect to at least one sensor mounted on the first and second clamping members. The real-time display 218 can be, e.g., an LED, a plurality of LEDs, and LCD, and the like. The at least one sensor mounted with respect to at least one of the first clamp member and the second clamp member can be, e.g., an ultrasound sensor, a Doppler ultrasound sensor, a pulse oximetry sensor, an infrared sensor, a light sensor, an IR sensor (an infrared proximity sensor), a stress/strain sensor and the like, and is effective to generate signals related to at least one anatomical parameter. The at least one anatomical parameter can be, e.g., a blood flow to a tumor, a blood flow to a tissue, a contour of at least a portion of the tumor, a contour of at least a portion of an organ, a contour of at least a portion of the tissue, an image of at least a portion of the tumor, an image of at least a portion of the organ, an image of at least a portion of the tissue, an oxygenation of tissue, a blood vessel location, a tissue composition, a disease composition, a proximity to surrounding tissue, a negative and/or positive surgical margin, and the like. Although illustrated as a real-time display 218 mounted to the handle section 206, in other exemplary embodiments, the real-time display 218 may be a stand-alone display such as, for example, a computer, a monitor, and the like, with wireless transmission of the information and/or signals. In addition (or alternatively) to the visual feedback provided by the real-time display 218, the exemplary clamping forceps 200 may include an audio feedback for the signals related to the at least one anatomical parameter, e.g., a beeping noise, an unprocessed Doppler signal, and the like. The exemplary clamping forceps 200 can be electrically powered by, e.g., connecting to an electrical socket, a battery, and the like. As illustrated in FIG. 3A, the clamping forceps 200 may be electrically powered by a rechargeable power pack battery 224.

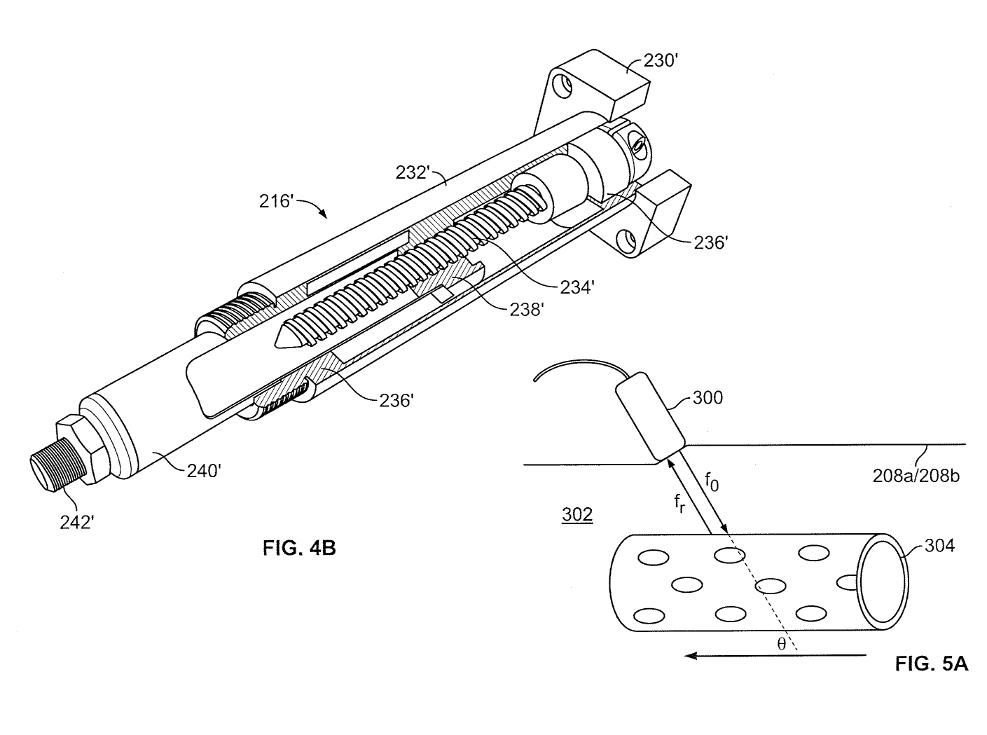

With reference to FIGS. 4A and B, an exemplary drive motor 216 is illustrated as a linear actuator motor. The drive motor 216 generally includes a base 230 for fixedly mounting the drive motor 216 relative to the handle section 206. The drive motor 216 generally further includes a rod-style stepper motor actuator 232, e.g., a stepper actuator, μ-stepper actuator, and the like, and a worm-gear 234. The worm-gear 234 can be, e.g., a threaded rod having variably sized threading to allow gross and/or fine actuation of the first and second clamp members. The worm-gear 234 can generally be movably connected to the rod-style stepper motor actuator 232 by a bearing housing 236. Further, the worm-gear 234 generally mechanically connects to the clamping mechanism 212 at a connector 238 by, e.g., interconnecting with the shaft 226 of the clamping mechanism 212. The rod-style stepper motor actuator 232 may include an integrated optical encoder 242 and encoder connections 240, i.e., wiring, for regulating, e.g., the speed, positioning, and the like, of the actuation of the drive motor 216, and thereby actuation of the first and second clamp members 208a and 208b.

With specific reference to FIG. 4B, an exemplary linear actuator drive motor 216′ is illustrated. (See, e.g., Tolomatic, ERD Electric Rod-Style Actuator, ERD15 and ERD 20 (2012)). In particular, the exemplary drive motor 216′ generally includes a base 230′, i.e., a motor mount, a tubular body 232′, and a worm gear 234′. The tubular body 232′ can be fabricated from, e.g., stainless steel, and the like. The drive motor 216′ generally further includes a nut 238′, e.g., a solid nut, a ball nut, and the like, for stabilizing and/or regulating the translation of the worm gear 234′. Bearings 236′ may be implemented to maintain a smooth operation of the drive motor 216′. A thrust tube 240′ can further be implemented in communication with the worm gear 234′ and can further be interconnected to the shaft 226 of the clamping mechanism 212 at the connector 242′. Thus, actuation of the worm gear 234′ results in actuation of the shaft 226 of the clamping mechanism 212 and further actuation of the first and second clamp members.

Although illustrated as a progressive worm gear clamping mechanism 212, in other exemplary embodiments, the clamping mechanism 212 can be, e.g., a spring-loaded mechanism, a gearing mechanism, a cable wire mechanism, a ratcheted mechanism, a motorized mechanism, a piezoelectric mechanism, a pneumatic mechanism, a solenoid actuator mechanism, a slide-crank mechanism, a slot yoke mechanism, a cam-style mechanism, a single scissor mechanism, a dual scissor mechanism, a gross and/or fine rack and pinion mechanism, an atraumatic cross spring mechanism, a linear spring mechanism, an electric cylinder actuator mechanism, a cable-pull drive mechanism, and the like. As discussed above and as will be discussed in greater detail below, the handle section 206, i.e., the user interface section, may generally also include, e.g., gross ratchet control, fine ratchet control, rotation control, articulation control, folding control of the first and second clamp members 208a and 208b, clamping actuation control, blood flow detection, imaging control, oxygenation sensing control, and the like. Further, the first and second clamp members 208a and 208b may be fabricated with and/or include, e.g., interconnected chain-link linkages, open-ended linkages, a saline and/or air balloon, malleable prongs, blood flow sensors, imaging sensors, oxygen sensors, high intensity focused ultrasound (HIFU) coagulation, bioresorbable material, and the like.

As discussed above, the exemplary clamping forceps 200 can generally include at least one sensor mounted with respect to at least one of the first clamp member 208a and the second clamp member 208b. Although discussed with respect to exemplary clamping forceps 200, it should be understood that other exemplary embodiments of the clamping forceps discussed herein may include the at least one sensor as described. The at least one sensor is generally effective to generate signals related to at least one anatomical parameter, e.g., a blood flow to a tumor, a blood flow to a tissue, a contour of at least a portion of the tumor, a contour of at least a portion of an organ, a contour of at least a portion of the tissue, an image of at least a portion of the tumor, an image of at least a portion of the organ, an image of at least a portion of the tissue, an oxygenation of tissue, a blood vessel location, a tissue composition, a disease composition, a proximity to surrounding tissue, a negative and/or positive surgical margin, a tissue density, a tissue biochemistry, a biomaterial composition, and the like. The at least one sensor can be mounted with respect to at least one of the first and second clamp members 208a and 208b such that, e.g., the clamping surface of the first and second clamp members 208a and 208b remains uniform, the at least one sensor can protrude slightly from the clamping surface plane of the first and second clamp members 208a and 208b, and the like. The first clamp member 208a and the second clamp member 208b generally also include at least one conduit, e.g., a passage, a cavity, a line, a hollow interior, lumen, and the like, for introduction of wiring for the at least one sensor. The wiring for the sensor generally passes through and/or to the first and second clamp members 208a and 208b, through the elongated body section 204 and into the handle section 206 where it connects to a visual and/or audio feedback device. In other exemplary embodiments, the wiring may pass through a conduit/lumen on the outside of e.g., the first and second clamp members 208a and 208b, the elongated body section 204, the handle section 206, and the like. As would be understood by those of ordinary skill in the art, the wiring and/or tubing located in the at least one conduit/lumen is generally flexibly positioned to permit folding and unfolding of the first and second clamp members 208a and 208b during insertion of the clamping forceps 200 into the surgical site through a trocar port/cannula.

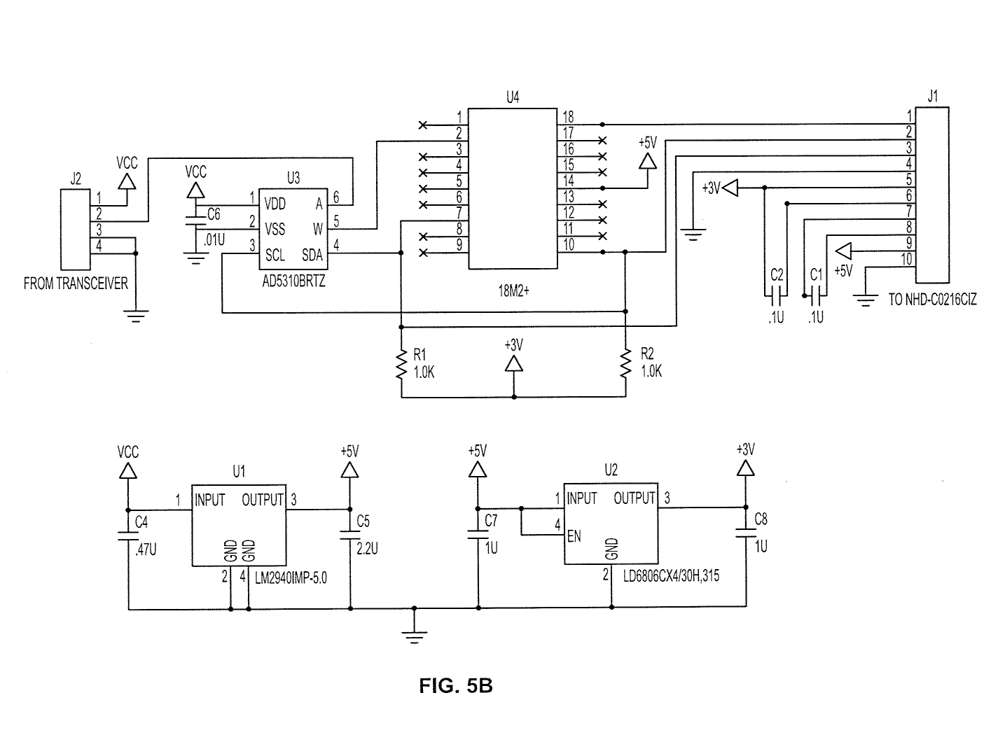

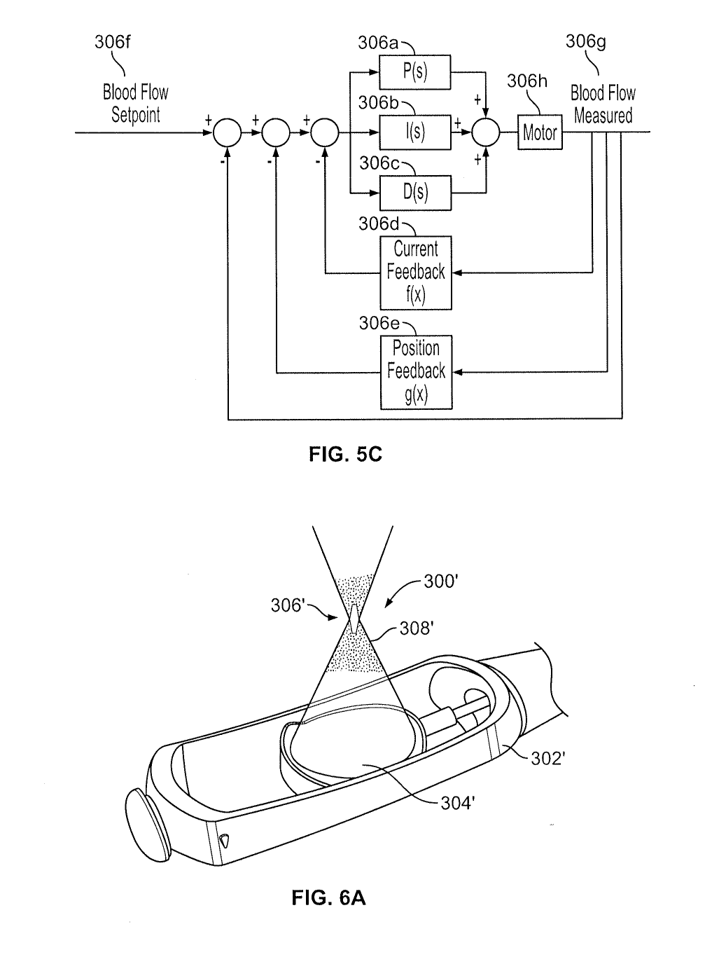

Turning to FIG. 5A, all exemplary sensor 300 is illustrated. The sensor 300 can be, e.g., an ultrasound sensor, a Doppler ultrasound sensor, a pulse oximetry sensor, an IR sensor, a biological sensor, a camera, a seed to delivery of therapy to the effected region, a photodiode light sensor, and the like. In particular, the ultrasound sensor and/or the Doppler ultrasound sensor may be implemented for detection of blood flow passing to the tumor. The pulse oximetry sensor may be implemented for detection of oxygen passing to, e.g., the organ, the tumor, the tissue, and the like. In some exemplary embodiments, the exemplary clamping forceps 200 may include means for injecting, e.g., a fluorescent dye, and the like, into a tumor and/or tissue at a surgical site. An exemplary IR sensor can further be implemented to detect the fluorescent dye in the tumor and/or tissue. In some exemplary embodiments, the photodiode light sensor may transmit light so as to combine with a light-sensitive drug for implementing photodynamic therapy (PDT) to, e.g., the organ, the tumor, the tissue, and the like. Although one sensor 300 is illustrated, it should be understood that in other embodiments, e.g., one, two, three, four, five, six, and the like, sensors 300 may be implemented depending on, e.g., the area of coverage for the signal from the sensor 300, and the like. For example, for a tumor of approximately 2.6 cm in diameter, a 3 cm diameter clamping member may be implemented with, e.g., three sensors 300, which are capable of obtaining signals to cover the entire 3 cm diameter surgical site. As a further example, for a tumor of approximate 1 cm in diameter, a 1.5 cm diameter clamping member may be implemented with, e.g., one sensor 300, which is capable of obtaining signals to cover the entire 1.5 cm diameter surgical site. Thus, as would be understood by those of ordinary skill in the art, the number of sensors 300 implemented may vary based on, e.g., the area of coverage for the signal from the sensor 300, the size of the tumor being excised, the size of the clamping members, and the like.

As illustrated in FIG. 5A, the sensor 300 is generally embedded into at least one of the first and second clamp members 208a and 208b such that the sensor 300 is positioned adjacent to the tissue 302 of the organ (or other structure) being clamped. The Doppler ultrasound and/or ultrasound sensor 300, i.e., a transceiver, generally produces an output signal f₀ at a predetermined angle θ, which passes through the tissue 302 and into the bloodstream 304, and receives a reflected return signal fᵣ, as depicted in FIG. 5A. The sensor 300 can be, e.g., an 8 MHz probe with a transceiver system. (See, e.g., VTI 8 MHz Surgical Doppler System, Vascular Technology Inc. (2012); and VTI 10 MHz Microvascular Doppler System, Vascular Technology Inc. (2012)). Based on the frequency of the reflected return signal fᵣ, the blood flow passing to the tumor or other anatomical location may be detected in real-time. Thus, as the user actuates the clamping mechanism 212 to clamp the first and second clamp members 208a and 208b around the tumor (or other structure), the sensor 300 can be implemented to detect and/or regulate the blood flow passing to the tumor (or other structure). For example, the user can continue clamping the first and second clamp members 208a and 208b around a tumor until the sensor 300 notifies the user that the blood flow to the tumor has been stopped. Thus, the organ can be sufficiently clamped to prevent blood flow to the tumor, while preventing the clamping forceps 200 from crushing and/or damaging the organ.

In some exemplary embodiments, an incoming Doppler audio signal, i.e., a reflected return signal fᵣ, is generally passed through a peak hold circuit to ensure sufficiently long hold times for ADC sampling. The peak hold circuit generally presents a sufficiently fast decay time to preserve the shape of the cardiac pulse while holding long enough to remove the high frequency artifacts of the Doppler signal. The Doppler signal is generally sampled by the microcontroller, which calculates and continues to monitor the pulse rate of the patient. A further calculation of the blood flow rate may be made based on the average magnitude of the sampled audio signal over the period of one cardiac cycle. The calculated percentage of blood flow (% BF) may be determined based on Equation 1 below:

$$\% BF = 1 - \frac{( {F}_{m} - {F}_{d} )}{( {F}_{m} - {F}_{0} )} \qquad ( 1 )$$

wherein Fₘ is the maximum flow rate detected, F_{d} is the detected flow rate, and F₀ is no flow rate. The % BF may be represented as, e.g., a percentage of the maximum blood flow rate detected during the surgical procedure.

With reference to FIGS. 5B and C, a blood flow detection schematic diagram and control diagram are provided. The control diagram of FIG. 5C generally includes a blood flow setpoint 306f, a motor 306h and a blood flow measured 306g. The control diagram further generally includes a proportional term 306a, i.e., P(s), of the control loop, an integral term 306b, i.e., l(s), of the control loop, and a derivative term 306c, i.e., D(s), of the control loop. Further, the control diagram generally includes current feedback 306d, i.e., f(x), a position feedback 306e, i.e., g(x), and a modulation wave generator, i.e., h(x) (not shown). The current feedback 306d can generally be a function that sets a desired blood flow setpoint 306f equal to the blood flow measured 306g to stop the motor 306h for over current errors based on the current sensor. The position feedback 306e can generally be a function that sets the desired blood flow setpoint 306f equal to the blood flow measured 306g to stop the motor 306h for out of range position errors based on an encoder feedback. The modulation wave generator can generally generate an instantaneous value based on the wave form created by a duty cycle and magnitude input by a surgeon.

Similarly, the sensor 300, e.g., a Doppler ultrasound sensor, an ultrasound sensor, and the like, can be utilized for obtaining an image of, e.g., at least a portion of the tumor, blood vessel location, and the like. The imaging provided from the sensor 300 generally aids in visualizing, e.g., the location of the tumor, the size of the tumor, the area required to be excised, the negative surgical margin to be attained, the required positioning of the first and second clamp members 208a and 208b, and the like. A pulse oximetry sensor 300 may be implemented for detecting the oxygenation of tissue. The pulse oximetry sensor 300 generally operates by passing wavelengths through the tissue 302 of the patient and to a photodetector (not shown). The change in absorbance of the wavelengths is generally measured to determine the oxygenation of the tissue. Thus, a surgeon can implement the pulse oximetry sensor 300 to, e.g., reduce the surgical margin (the amount of healthy tissue 302 being excised with the tumor), generate feedback for photodynamic therapy by providing a signal of when there is a reduction and/or no oxygen in the tissue and instruct the system and/or surgeon to adjust the light's intensity, frequency, wavelength, time duration, and/or stop shinning further light, and the like.

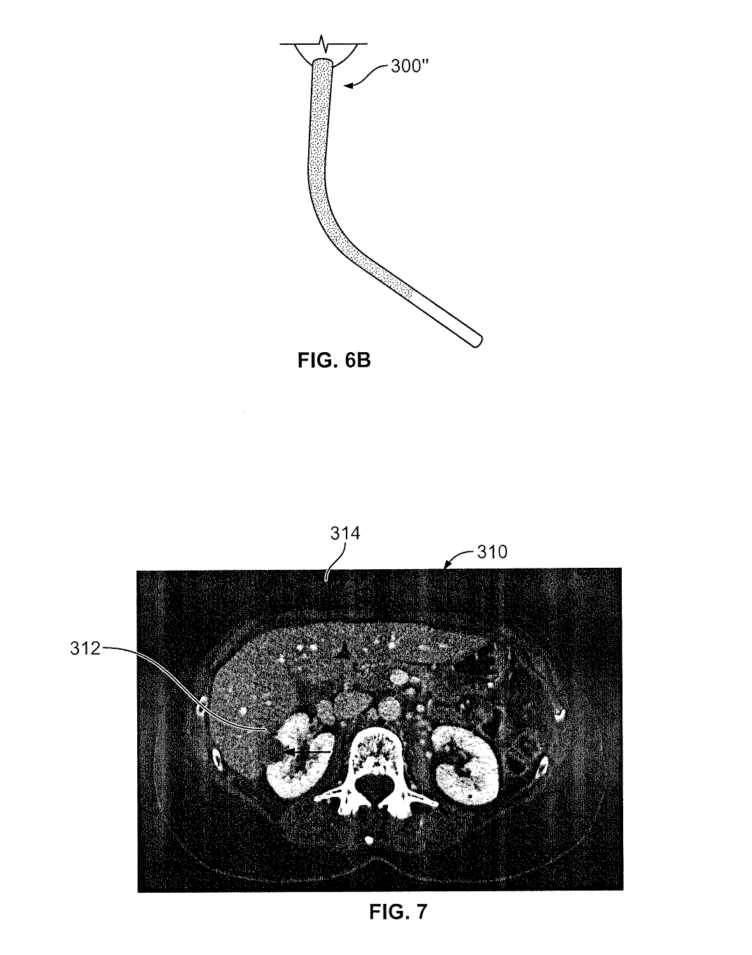

With reference to FIG. 6A, in some exemplary embodiments, the sensor 300′ can be a high-frequency ultrasound sensor 300′. The high-frequency ultrasound sensor 300′ generally includes a sensor body 302′ and a transducer 304′ for producing and/or transmitting the high frequency ultrasound waves 308′, e.g., approximately between 1 and 10 MHz, and an intensity of up to approximately 20 W/cm². In some exemplary embodiments, the intensity can be up to approximately 1,000 W/cm² for, e.g., ablation applications, and the like. The transducer 304′ generally transmits the high frequency ultrasound waves 308′ such that a focal point 306′ is created. The focal point 306′ can be adjusted such that the focus of the high frequency ultrasound waves 308′ is on the area of tissue to be treated. The high frequency ultrasound waves 308′ may be implemented to, e.g., treat a tumor, coagulate tissue for a period of time during the surgical procedure, ablate tissue, create a region of constricting and/or swelling tissue so as to create a region blocked from blood flow, and the like. In other exemplary embodiments, a photodiode 300″ may be utilized as a form of therapy for tissue to be treated, as illustrated in FIG. 6B.

Although not illustrated, in other exemplary embodiments, clamping forceps 200 may include means for providing alternative therapeutic treatments to at least one of a tumor, tissue surrounding a tumor or other tissue, e.g., at least one needle, at least one sensor, at least one probe, and the like, in cooperation with the first and second clamp members 208a and 208b. The alternative therapeutic treatments can be at least one of, e.g., a tissue excision, a hemostatic treatment, an RF therapy, a thermal treatment, a cryogenic treatment, a brachytherapy, a radiation therapy, an application of a therapeutic agent, a pharmaceutical agent, a genomic agent, and the like. Further, in other exemplary embodiments, the clamping forceps may include, e.g., a blade, an ablation instrument, a grinding mechanism, a suction mechanism, and the like, in cooperation with the first and second clamp members 208a and 208b.

With reference to FIG. 7, a CT scan 310 of a cross-section of a patient is illustrated. In particular, the CT scan 310 provides an image of an organ 314 with the contour of a tumor 312 indicated by the arrow. The CT scan 310 generally can be utilized as a visual reference by a user to, e.g., properly position the first and second clamp members 208a and 208b such that the tumor 312 is sufficiently surrounded by the inner perimeter of the first and second clamp members 208a and 208b prior to and/or after clamping, visualize the size of the tumor 312 to ensure that the tumor 312 is fully excised, visualize the location of blood vessels and/or healthy tissue to prevent damage to said blood vessels and/or healthy tissue, and the like. In some exemplary embodiments, the CT scan 310 may be utilized to obtain an initial position of the tumor 312, and the exemplary sensors 300 of the first and second clamp members 208a and 208b may further be implemented to accurately navigate and/or position the first and second clamp members 208a and 208b around the tumor 312. In particular, the sensors 300 may be implemented in real-time intra-operatively during a surgical procedure to, e.g., determine the contour of a tumor 312, position the first and second clamp members 208a and 208b around the organ 314 and/or tumor 312, to measure and/or manage the negative surgical margin intra-operatively. In some exemplary embodiments, images creates by a plurality of ultrasound sensors 300 may be “stitched” together in order to produce a comprehensive image of the desired area and can further aid a surgeon in determining, e.g., the boundary of the surgical margin, and the like.

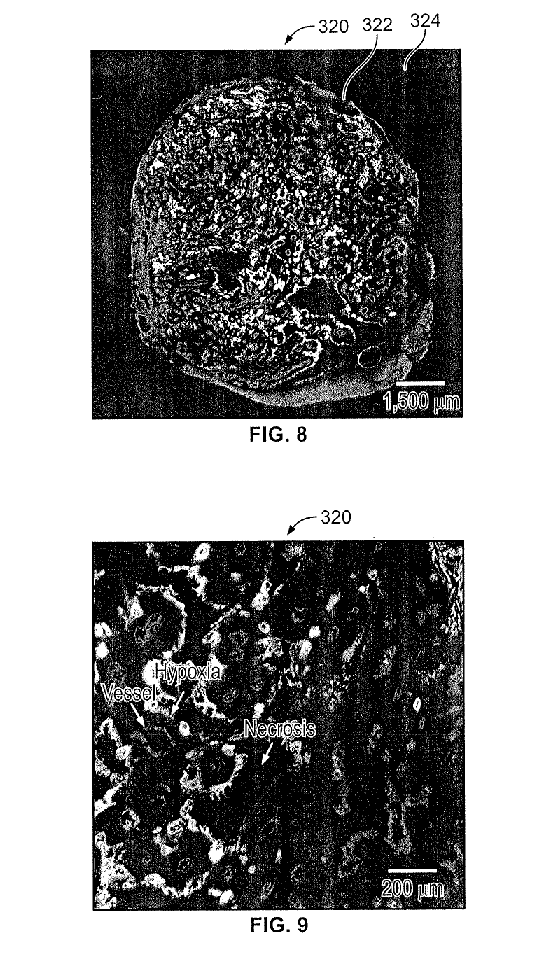

With reference to FIG. 8, an exemplary cross-sectional tumor contour image 320 is provided as obtained by a sensor 300, e.g., an ultrasound sensor, a Doppler ultrasound sensor, a pulse oximetry sensor, and the like. In particular, the tumor contour image 320 illustrates the tumor 322 and the surrounding tissue 324. In some exemplary embodiments, the sensor 300 may be utilized to obtain, e.g., a three dimensional image of the tumor, a three dimensional image of the organ, a three dimensional image of the tissue surrounding the tumor, and the like. In addition, with the aid of the pulse oximetry sensor 300, the tumor contour image 320 further provides visual feedback of the oxygenation of tissue in the tumor 322. FIG. 9 shows a higher magnification of the tumor contour image 320 and, in particular, illustrates the tissue characteristics and oxygenation of the tissue in the tumor 322, e.g., a histology image of various tissue types. As indicated by the arrows of FIG. 9, the sensor 300 provides a visual image which shows the location of blood vessels, the areas undergoing hypoxia, i.e., tissue deprived of oxygen, and the areas undergoing necrosis, i.e., the death of cells of the tissue. Thus, a user can monitor the oxygenation of tissue surrounding the tumor and/or of the tumor during the surgical procedure to reduce the surgical margin. Separate from and/or in combination with the visual feedback, other exemplary embodiments of the clamping forceps 200 generally include a real-time audio feedback, e.g., a beeping, to indicate the at least one anatomical parameter being sensed.

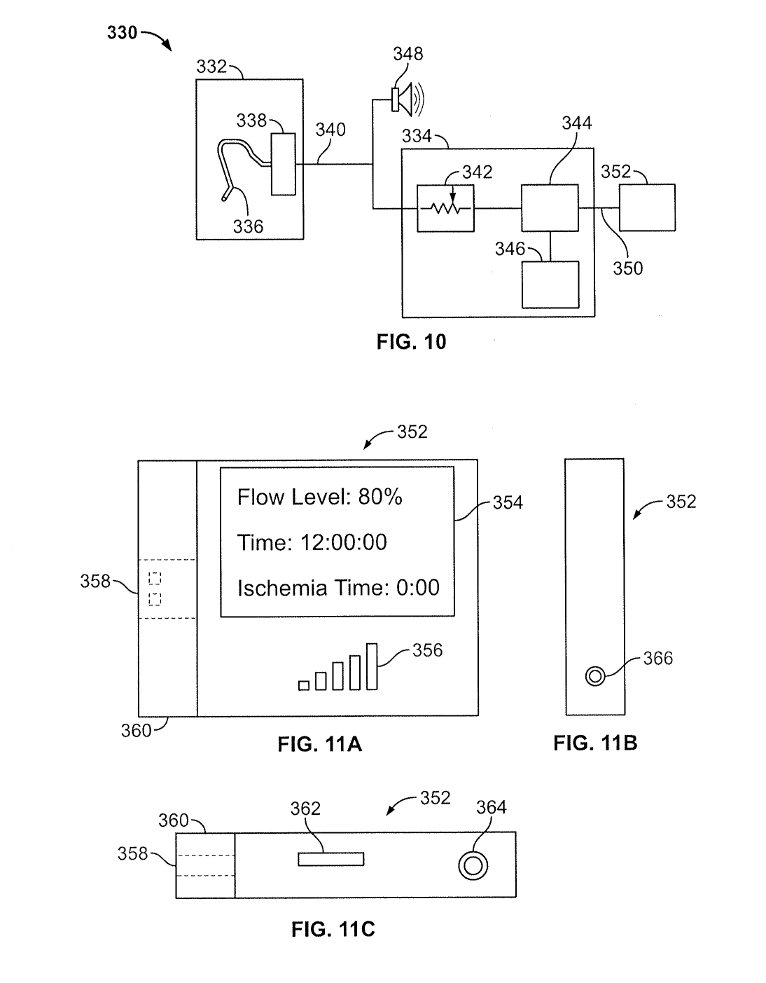

Turning now to FIG. 10, an exemplary control diagram for a sensor control system 330 is provided. Although the control diagram of FIG. 10 is directed to a sensor control system 330 for a Doppler ultrasound sensor 300, in other exemplary embodiments, the sensor control system 330 may be implemented with other sensors 300, e.g., an ultrasound sensor, a pulse oximetry sensor, and the like. The sensor control system 330 generally includes a sensor system 332, e.g., a Doppler sensor system, and a control system 334. The sensor system 332 generally includes a probe 336 and a transceiver and/or transducer 338 for generating and/or receiving signals. The transceiver and/or transducer 338 can further generate an audio output signal 340 to provide real-time audio feedback 348 to the user with respect to the anatomical parameter being measured.

The control system 334 generally includes an automatic level control 342, a signal processing microcontroller (MCU) 344, and a data storage 346, e.g., a database. The control system 334 further generally processes the signals from the transceiver and/or transducer 338 and generates a visual output signal 350 to provide real-time video feedback 352 to the user with respect to the anatomical parameter being measured. The automatic level control 342 can be of the type generally used in the industry to control analog signals in audio signal processing systems. The automatic level control 342 generally reduces and/or prevents noise from saturating the control circuit and can adjust automatically to make the algorithm applicable over a wide range of signal intensities. For example, in some exemplary embodiments, the level control can generally be accomplished with a voltage divider controlled by a digital potentiometer.

The MCU 344 of the control system 334 can generally be, e.g., a low cost, low power MCU with sufficient analog input sensitivity to sample the signal from the level control circuit of the automatic level control 342. The MCU 344 generally calculates and/or tracks the anatomical parameter being measured, e.g., the blood flow levels, based on the incoming Doppler signal. For example, the MCU 344 measures blood flow by tracking the approximate integral of the low frequency signal, i.e., the patient's pulse, using a rectangular approximation method based on the signal magnitude and the known sampling time. The MCU 344 further generally functions to, e.g., automatically adjust the level control circuit of the automatic level control 342, store data in the data storage 346, output visual feedback 352 with a visual output signal 350, and the like.

The data storage 346 of the control system 334 generally stores data relating to the anatomical parameter being measured and/or monitored, e.g., the blood flow level, during the surgical procedure. The frequency of obtaining data signals, i.e., the data density, can be at any desired frequency depending on, e.g., the anatomical parameter being monitored, the type of surgical procedure being performed, and the like. In general, the data can be output in a user-friendly format, e.g., a .csv file, which can be opened as a spreadsheet. The data can further be combined with, e.g., a Visual Basic (VB) script, and the like, to display the data in a meaningful manner to the user. The data storage 346 can implement, e.g., flash memory card data storage, and the like, for storing the data during the surgical procedure. As would be understood by those of ordinary skill in the art, the data collected during the surgical procedure may be stored in the data storage 346 indefinitely or may be stored for a predetermined period of time during and/or after the surgical procedure.

The visual feedback 352 generated from the visual output signal 350 can aid the user, i.e., the surgeon, by providing all alternative platform to the audio feedback 348 for estimating the blood flow level. Thus, rather than regulating the blood flow to the tumor based on the progressive audio feedback 348, the user may visually monitor the level of blood flow in the surgical site. The visual feedback 352 can be at least one of, e.g., a flow/no flow LED, a series of LEDs indicating the flow level, an LCD display providing more detailed feedback (for example, 100%, 75%, 50%, 25% and 0% rates of blood flow), and the like. As would be understood by those of ordinary skill in the art, the exemplary control system 334 generally controls the clamping force between the first and second clamp members 208a and 208b such that, e.g., blood flow to the surgical site is regulated as desired, while the organ has not been crushed and/or damaged. In some exemplary embodiments, e.g., powered clamping forceps, the signal from the probe 336 may be transmitted to a drive motor which facilitates a substantially one-step operation. For example, the surgeon may actuate the probe 336 and the motor generally determines the precise pressure to exhibit on an organ.

FIGS. 11A-C illustrate an exemplary visual feedback 352, which includes an LCD detailed display 354 and an LED display 356. In some exemplary embodiments, the display 354 may be, e.g., an LED display panel, and the like. In particular, FIG. 11A is a front view, FIG. 11B is a bottom view, and FIG. 11C is a side view of the exemplary visual feedback 352. The LCD detailed display 354 generally includes, e.g., the blood flow level in terms of percentage, the time, the ischemia time, a graph of the cardiac cycle, an indication of whether the first and second clamp members 208a and 208b have been locked in the desired clamping position by a locking mechanism, and the like. The LED display 356, i.e., a one-glance LED feedback display, can be defined by, e.g., a plurality of independent bars which are lit to indicate the level of flow detected. As would be understood by those of ordinary skill in the art, as the blood flow detected is reduced due to a greater clamping force of the first and second clamp members 208a and 208b, the appropriate bars can be unlit to provide the representative visual feedback to the user. The exemplary visual feedback 352 generally further includes, e.g., a removable cover 360, an electrical output connection 358, e.g., a USB connection, a removable media slot 362, a power connection 364, e.g., a DC power input, an audio input 366, and the like. In some exemplary embodiments, a detailed data log may be implemented to record data, e.g., any and all errors, time stamps, other vital information, and the like, which may be downloaded through a USB/SD output port after the surgical procedure.

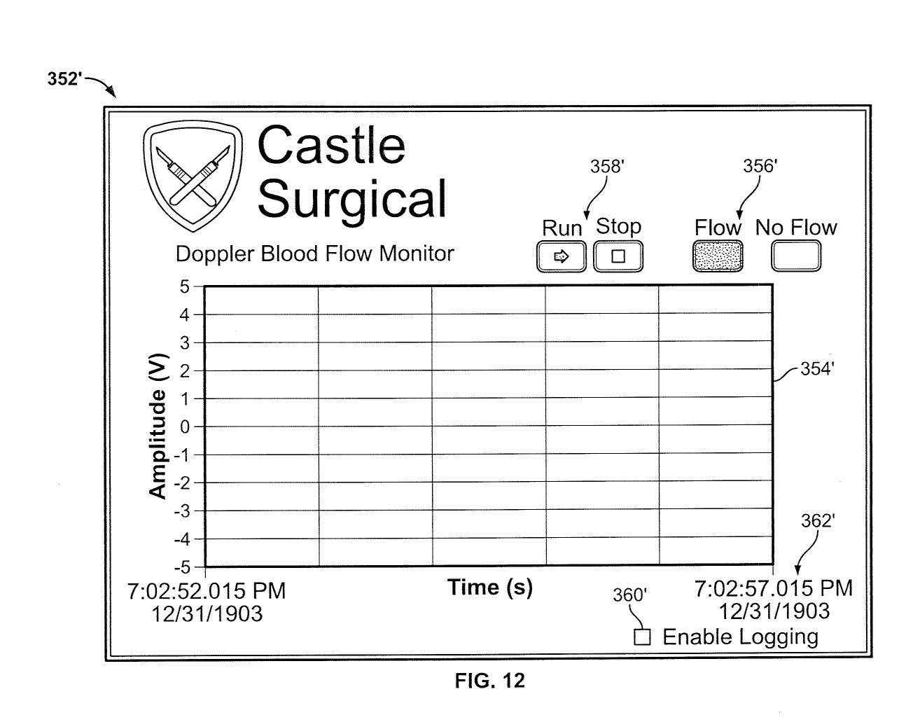

With reference to FIG. 12, an alternative exemplary visual feedback 352′ is provided that generally includes an LCD display 354′ and an LED display 356′. The LCD display 354′ may be defined by a real-time graph of the amplitude versus the time in seconds of the blood flow being detected. Thus, a user can monitor the level of blood flow being detected in order to regulate the level of blood flow desired for the surgical operation. The LED display 356′ generally includes two LED indicators for flow and no flow. As would be understood by those of ordinary skill in the art, when blood flow is detected, the “flow” LED can be lit and, in contrast, when no blood flow is detected, the “no flow” LED can be lit. Thus, the user is provided with a simple indicator of whether blood flow has been detected by the sensor. The exemplary visual feedback 352′ can further include, e.g., a run/stop control 358′, i.e., a control for running and stopping the visual feedback 352′, a logging selector 360′ to indicate that the output data should be collected and stored in the data storage 346, a date/time field 362′ indicating the date and time of the surgical procedure, and the like.

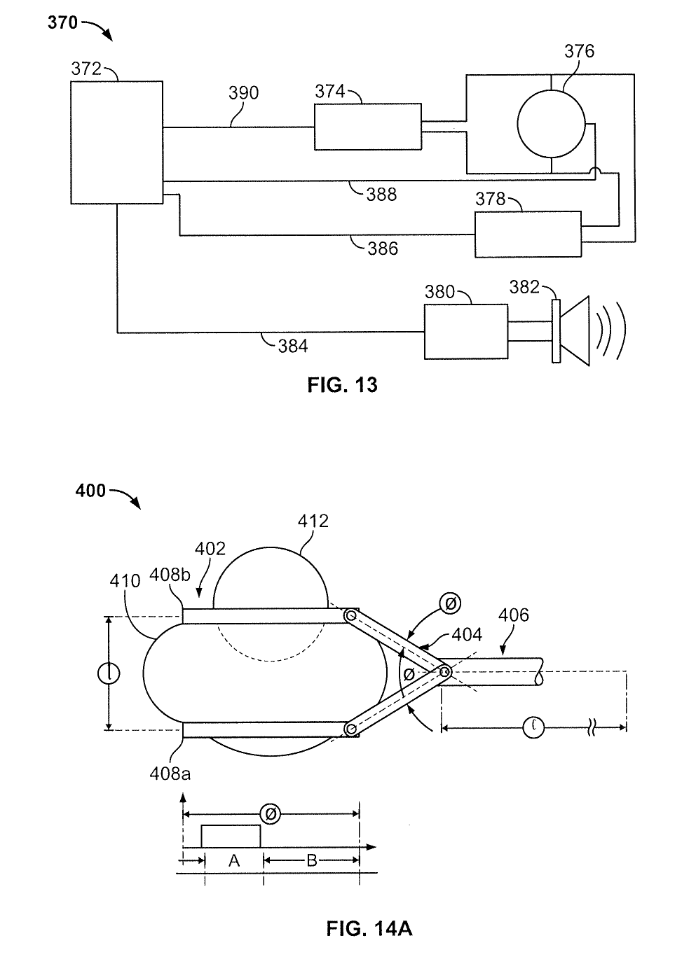

Turning now to FIG. 13, an alternative exemplary sensor control system 370 is illustrated, generally including a signal processing microcontroller (MCU) 372, a drive motor 376, and a sensor 382, e.g., a Doppler ultrasound sensor. The exemplary sensor control system 370 generally further includes a speed controller 374, a current monitor 378 and a sensor controller 380, e.g., a Doppler ultrasound sensor controller. The MCU 372 and the sensor 382 are generally substantially similarly in structure and/or function as the MCU 344 and the probe 336 depicted in FIG. 10, unless discussed herein. The sensor controller 380 generally functions to control and/or regulate the sensor 382. Thus, a Doppler ultrasound sensor controller 380 regulates the Doppler probe sensor 382 appropriately to maintain a steady blood flow detection reading. The sensor controller 380 generally further functions to generate a sensor feedback signal 384, e.g., a blood flow feedback signal, to the MCU 372.

In response to the sensor feedback signal 384, the clamping force of the first and second clamp members 208a and 208b actuated by the drive motor 376 can be regulated, e.g., manually, automatically, and the like, by the MCU 372. For example, the MCU 372 generally regulates the speed of the drive motor 376 by actuating the speed controller 374 with a motor control signal 390. Thus, the MCU 372 can generate a motor control signal 390 to, e.g., increase the clamping action, decrease the clamping action, and the like, of the first and second clamp members 208a and 208b. In response, the drive motor 376 generally generates a speed feedback signal 388 and a torque feedback signal 386 through a current monitor 378. Based on the speed feedback signal 388, the torque feedback signal 386, and/or the sensor feedback signal 384, the MCU 372 generally regulates the clamping action of the first and second clamp members 208a and 208b.

In some exemplary embodiments, the MCU 372 can generate real-time feedback, e.g., visual feedback, audio feedback, and the like, to indicate to the user that the anatomical parameter being monitored requires a form of action from the user, e.g., the Doppler ultrasound detects a blood flow and requires the first and second clamp members 208a and 208b to be clamped with greater force. In other exemplary embodiments, the MCU 372 can automatically adjust the clamping force applied with respect to at least a portion of at least one of the first and second clamp members 208a and 208b. Thus, rather than requiring an action from the user, the MCU 372 automatically adjusts the clamping force applied to the organ to prevent blood flow from reaching the surgical site, while preventing damage to the organ. The automatic adjustment of the clamping force may be used, e.g., to initially clamp the first and second clamp members 208a and 208b around an organ prior to a surgical procedure, to maintain a clamping pressure during a surgical procedure, and the like. For example, the blood flow rate sensed by a sensor may be implemented as a set point in a PID loop to control the speed of the clamping mechanism 212, i.e., the clamping motor. The feedback error for blood flow may be calculated by, e.g., subtracting the desired blood flow rate, i.e., zero for a fully clamped position, from the calculated and/or sensed blood flow rate. This feedback error may be used to set the desired speed for clamping actuation by the clamping mechanism 212. Thus, this desired speed may be set as the speed for clamping mechanism 212.

As would be understood by those of ordinary skill in the art, the automatic adjustment of the clamping force may be of further substantial help during a surgical procedure where the organ being operated on, e.g., deflates, loses blood, loses tissue, and the like, and thereby reduces in thickness, a situation that generally requires immediate action at the surgical site. For example, although an initial clamping force may be sufficient at the beginning of a surgical procedure to prevent blood flow to the surgical site, once a kidney has been excised, a greater clamping force may be required to adjust the clamping pressure and/or maintain the prevention of blood flow to the surgical site. Thus, based on the sensor feedback signal 384 generated by the sensor controller 380 to the MCU 372, the MCU 372 can automatically control the clamping force of the first and second clamp members 208a and 208b by regulating the drive motor 376 in order to maintain the desired clamping force around the organ and/or the surgical site. In some exemplary embodiments, a manual override generally allows a user to bypass the automatic adjustment of clamping force when necessary. In further exemplary embodiments, a system override may be provided for stopping the speed control of the drive motor 216 based on a current sensor which generally monitors the current draw of the drive motor 216. If the drive motor 216 is determined to be over a predetermined current, the drive motor 216 may be stopped to prevent an unsafe condition for the patient. A position sensor may further monitor the position of the first and second clamp members 208a and 208b such that if the position of said clamp members is found to be out of range and/or the speed calculated from the position data varies from the predetermined and/or set speed, the drive motor 216 may be stopped.

The exemplary sensor control systems 330 and/or 370 may further be implemented in conjunction with a transient ischemia form of exemplary clamping forceps 400. As would be understood by those of ordinary skill in the art, rather than blocking blood flow to a tumor completely, it may be advantageous to variably control where and/or how much blood is allowed to flow during a surgical procedure into the area of tumor excision. In particular, it should be understood that blood perfusion generally maintains healthy tissue. Thus, it would be advantageous to regulate a desired and/or predetermined amount of blood perfusion through the tissue directly around the area which is being excised in order to reduce the surgical margin, i.e., keep more of the healthy tissue of the organ for the patient, while still being able to complete the surgical excision of tumor 412 during the surgical procedure, e.g., partial nephrectomy, partial hepatectomy, and the like.

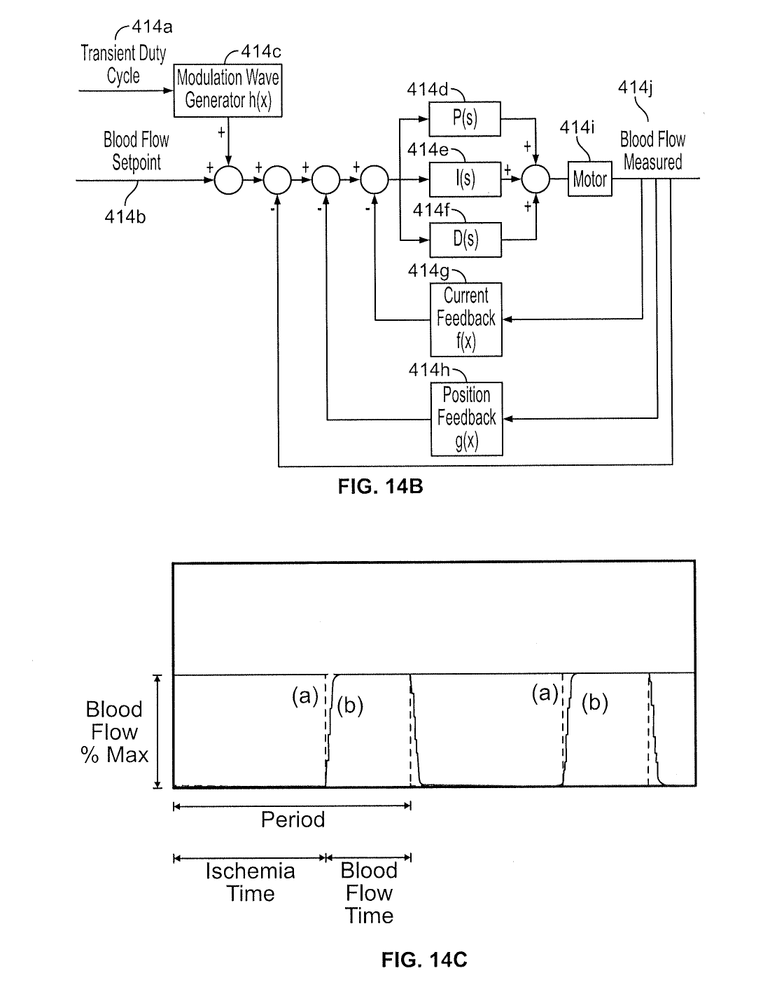

The exemplary clamping forceps 400 shown in FIG. 14A are substantially similar in structure and/or function to the clamping forceps 100 and 200 discussed above. In particular, clamping forceps 400 generally includes a head section 402, a clamping mechanism 404, and an elongated body section 406. The head section 402 generally includes first and second clamp members 408a and 408b, i.e., posterior and anterior clamp members, respectively. In particular, the first and second clamp members 408a and 408b are generally configured and dimensioned to be positioned around an organ 410 such that the tumor 412 to be excised is positioned within the inner perimeter of the first and second clamp members 408a and 408b. The clamping forceps 400 further includes a control mechanism (not shown) for variably controlling the clamping force applied with respect to at least a portion of at least one of the first clamp member 408a and the second clamp member 408b. The first and second clamp members 408a and 408b may be manufactured as, e.g., a plurality of interconnected linkages, a topographically variable clamping member, and the like, such that a user can regulate the amount of clamping pressure applied around the periphery of the first and second clamp members 408a and 408b. In particular, the variable clamping force applied with respect to at least one of the first clamp member 408a and the second clamp member 408b is generally effective to permit controlled blood flow to tumor 412 (or other structure) between the first clamp member 408a and the second clamp member 408b based on a controlled reduced clamping force in a region of controlled blood flow. Thus, the controlled blood flow to the tumor 412 can be at least one of, e.g., an intermittent blood flow, a moderated blood flow, a localized blood flow, a fully unrestricted blood flow, a fully restricted blood flow, and the like.

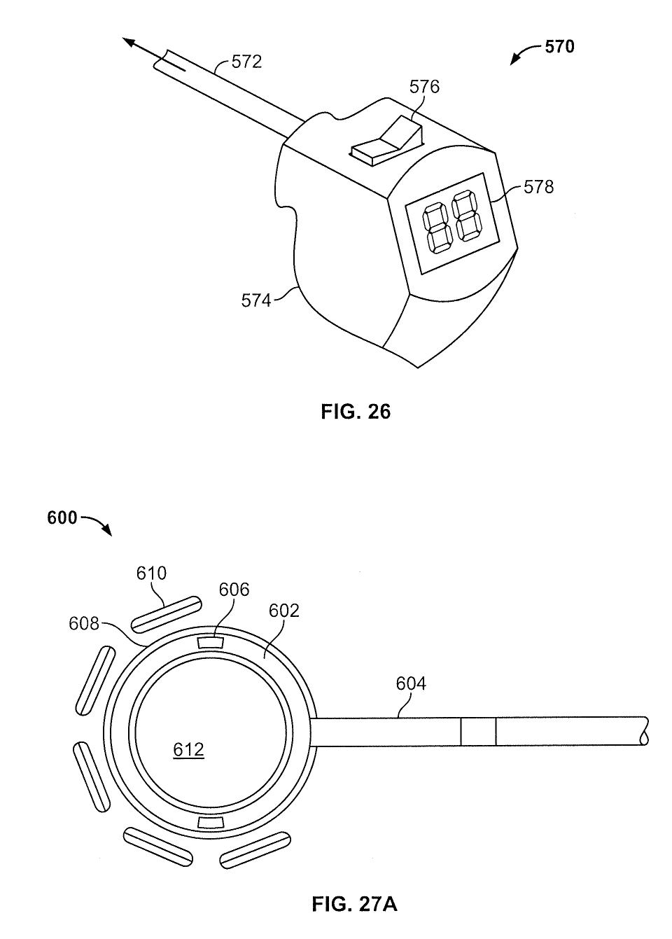

For example, the first and second clamp members 408a and 408b may be regulated such that area “A” of the organ 410 is clamped with a clamping force of approximately 25% of the full force of the clamping mechanism 404 and area “B” of the organ 410 is clamped with a clamping force of approximately 100% of the full force of the clamping mechanism 404. Thus, the reduced controlled clamping force in area “A” allows a controlled blood flow to pass through the tissue in the surgical site within the perimeter of the first and second clamp members 408a and 408b, thereby generally preserving this tissue from excision. As an additional example, the first and second clamp members 408a and 408b may be regulated such that area “A” of the organ 410 is clamped with a clamping force of approximately 85% of the full force of the clamping mechanism 404 and area “B” of the organ 410 is clamped with a clamping force of approximately 35% of the full force of the clamping mechanism 404. Although illustrated as parallel first and second clamp members 408a and 408b, it should be understood that in other exemplary embodiments, the first and second clamp members 408a and 408b may be, e.g., angled, rounded, variably positioned, and the like, relative to each other based on the variable clamping force being applied. In other exemplary embodiments, the variable clamping force, i.e., the percentage of the full clamping force of the clamping mechanism 404, generally varies in the range of approximately 0% to 100% clamping force. In addition, in other exemplary embodiments, the areas “A” and “B” can vary around the perimeter of the first and second clamp members 408a and 408b such that, e.g., one, two, three, four, five, and the like, variable clamping forces may be applied around the periphery of the first and second clamp members 408a and 408b. In some exemplary embodiments, the clamping mechanism 404 may be implemented on a duty-cycle set by a user interface, e.g., the toggle switch system 570 of FIG. 26, to moderate the clamping pressure over time for transient ischemia designs discussed in detail below.

The variable clamping force may be regulated, e.g., by exemplary sensor control systems 330 and/or 370, manually by a user by actuation of controls located at the handle section, a preset duty cycle, a waveform modulation, and the like. In some exemplary embodiments, the surgeon may be permitted to adjust, e.g., the maximum level of allowed blood flow, the period length of a cycle, the duty cycle as a ration of time with blood flow to time under ischemia, and the like. As illustrated in FIG. 14A, ø represents the modulating duty cycle of the clamping forceps 400 which indicates the percentage of time full-clamping force is being applied to a periphery of tissue so as to block blood flow to the tumor 412, τ represents the clamping distance between the first and second clamp members 408a and 408b based on the clamping force being applied, θ represents the clamping angle of the clamping mechanism 404 components based on the clamping force being applied, and x represents the telescoping distance and/or position of the elongated body section 406 relative to the handle section (not shown).

With reference to FIG. 14B, an exemplary control system diagram for a transient ischemia clamping forceps is provided. The exemplary control system generally includes a transient duty cycle 414a, a blood flow setpoint 414b, a motor 414i and a blood flow measured 414j. The exemplary control system generally further includes a proportional term 414d, i.e., P(s), an integral term 414e, i.e., l(s), and a derivative term 414f, i.e., D(s), of the control loop. In general, the exemplary control system further includes a current feedback 414g, i.e., f(x), a position feedback 414h, i.e., g(x), and a modulation wave generator 414c, i.e., h(x). The current feedback 414g can generally be a function which sets the desired blood flow setpoint 414b equal to the blood flow measured 414j to stop the motor 414i for over current errors based on a current sensor. The position feedback 414h can generally be a function which sets the desired blood flow setpoint 414b equal to the blood flow measured 414j to stop the motor 414i for out of range position errors based on a rotary encoder feedback. The modulation wave generator 414c can generally be an instantaneous value based on the wave form created by the duty cycle and magnitude input by the surgeon.

With reference to FIG. 14C, an exemplary resultant output waveform diagram is provided for a transient ischemia system. In particular, the duty cycle in FIG. 14C is generally set by a user and enabled blood to flow in a controlled manner. As an indication of the system's accuracy, waveform “a” represents an input form the surgeon and waveform “b” represents the actual clamping forceps response to facilitate the surgeon's demand. Once the waveform has been generated and the transient ischemia mode has been initiated, the set point generally varies with time equal to the value of the waveform. In general, calculated errors may be reset when the set point transitions in order to avoid drastically changing the integral term 414e and the derivative term 414f of the control scheme of FIG. 14B. As a safety factor, in some exemplary embodiments, an error may cause the exemplary clamping forceps to cease functioning in transient ischemia mode and the clamping forceps can transition to a the safest position, e.g., a fully clamped position.

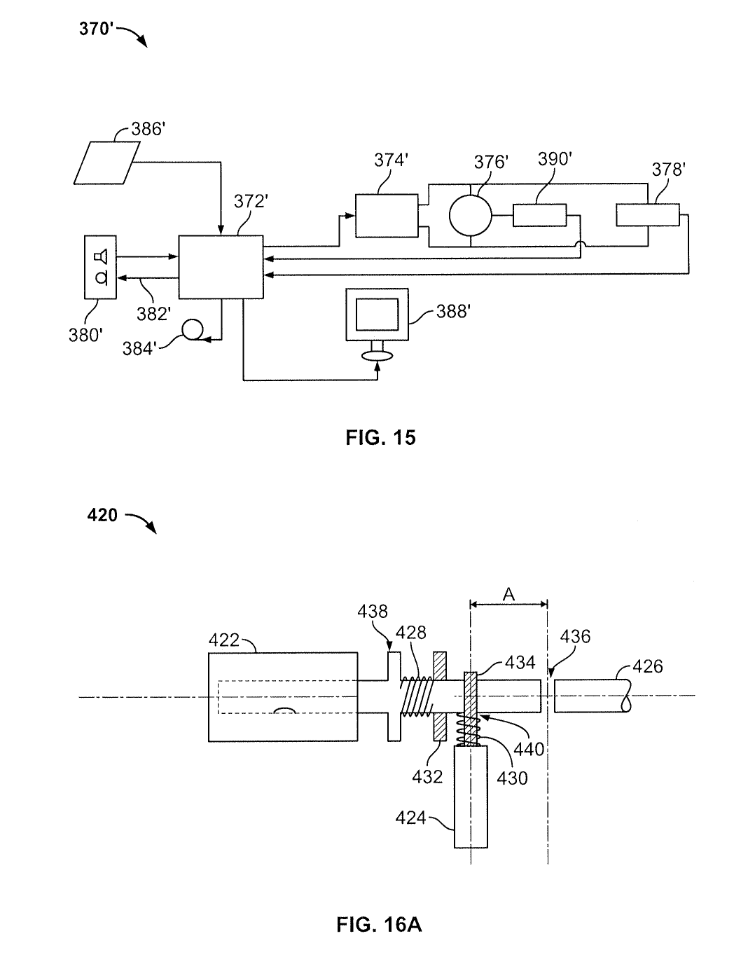

Turning now to FIG. 15, an alternative exemplary sensor control system 370′ is illustrated, generally including a signal processing microcontroller (MCU) 372′, a drive motor 376′, and a sensor 380′. The sensor 380′ generally receives an input signal 382′, e.g., a control signal, from the MCU 372′ and returns an output signal 382′, e.g., a blood flow detection signal, to the MCU 372′ for further action. Based upon the output signal 382′ from the sensor 380′, the MCU 372′ generally generates a motor control signal to the motor controller 374′ in order to actuate the drive motor 376′ to regulate, e.g., increase the clamping force, decrease the clamping force, and the like, of the first and second clamp members 208a and 208b. A rotary encoder 390′, i.e., a shaft encoder, may be implemented to convert the angular position and/or motion of the clamping mechanism 212 to indicate the clamping force being applied with respect to the first and second clamp members 408a and 408b. A current sensor 378′ can generally be implemented to generate a signal to the MCU 372′ based on the actuation of the drive motor 376′ to determine whether further clamping action is required and subsequently applied. Based on the signals generated by the sensor 380′, the rotary encoder 390′, and/or the current sensor 378′, the MCU 372′ generally determines the degree to which regulation of the clamping force created by the first and second clamp members 208a and 208b is required. Actuation of the drive motor 376′ may be made automatically by the MCU 372′ in response to the signals received and/or manually by user input through the user interface 186′, i.e., an operator interface. The user interface 186′ can be, e.g., an actuator and/or trigger on the handle section 206, and the like. A display 388′ may generate real-time visual and/or audio feedback to the user with respect to an anatomical parameter being monitored, e.g., blood flow to the tumor. In some exemplary embodiments, the display 388′ may be a touch-screen so as to enable the user to implement changes in, e.g., a duty cycle, and the like. Further, the data collected during the surgical procedure for the anatomical parameter being monitored may be stored in a data storage 384′, e.g., a database, for further processing and/or transmitted wirelessly to an external receiver (not shown) via, e.g., Bluetooth, any methods standard in the art of wireless communication, and the like.

In accordance with embodiments of the present disclosure, the exemplary clamping forceps discussed herein may be actuated by one or more solenoid actuation mechanisms. In particular, the solenoid actuation mechanism(s) generally define a clamping mechanism and transmit, via a solenoid, power presented by the user through the user interface, e.g., the handle section, to the head section in order to deliver and regulate the clamping force between the first and second clamp members 208a and 208b. The exemplary solenoids may be, e.g., a single-pole solenoid, a dual-pole solenoid, a variable-force solenoid, and the like. (See, e.g., Woodward Solenoids, Solenoid Components for Control Systems (2012)).

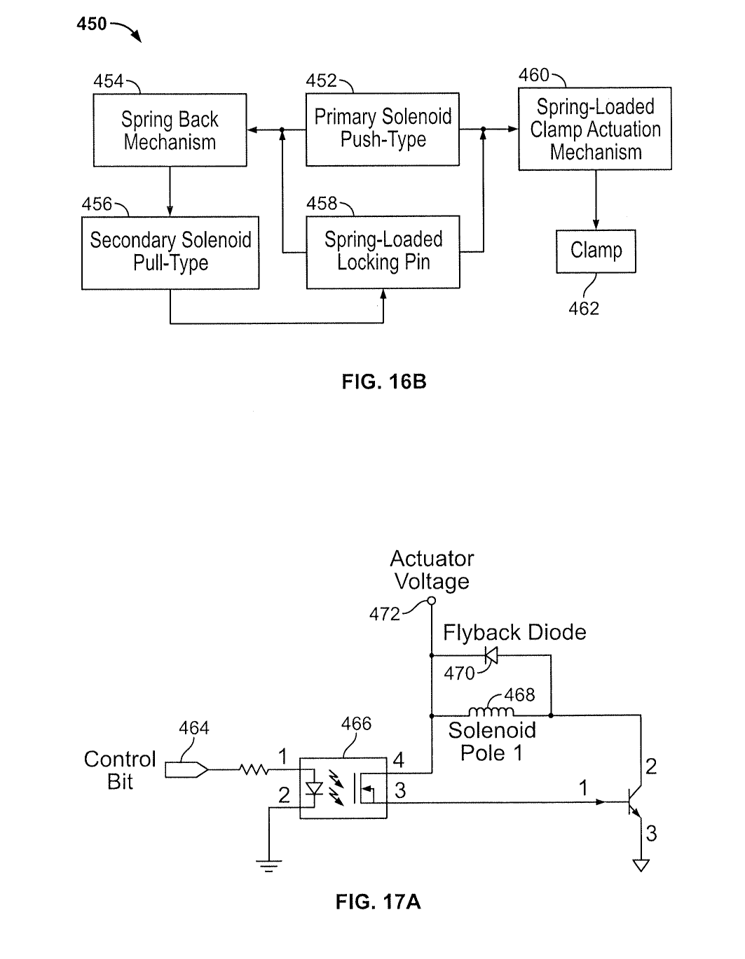

An exemplary single-pole solenoid actuation mechanism 420 is illustrated in FIG. 16A for a clamping forceps according to the present disclosure in a clamp open position. The single-pole solenoid actuation mechanism 420 generally includes a primary solenoid 422, e.g., a push-type solenoid, and a secondary solenoid 424, e.g., a pull-type solenoid. The connector shaft 426 generally extends from the single-pole solenoid actuation mechanism 420 and is in mechanical communication with the first and second clamp members 208a and 208b. The single-pole solenoid actuation mechanism 420 generally further includes a push-back spring 428, a pull-back spring 430, a stationary spring 432, and an actuating bushing 438. The locking pin 434 of the single-pole solenoid actuation mechanism 420 may be positioned in a first slot 440, i.e., the open clamp position, and a second slot 436, i.e., a closed clamp position. As would be understood by those of ordinary skill in the art, the single-pole solenoid actuation mechanism 420 can be actuated into either the closed or open clamp position, which in turn regulates the clamping force of the first and second clamp members 208a and 208b around an organ or other structure.

With reference to FIG. 16B, a flow chart 450 of a single-pole solenoid actuation mechanism 420 is provided. In particular, the spring back mechanism 454 generally actuates the pull-back spring 430 of the secondary solenoid 456, e.g., a pull-type solenoid, into a compressed or expanded position, i.e., the spring-loaded locking pin 458 is actuated into a locked position or an unlocked position. In the expanded/pulled back position, the pull-back spring 430 pulls back the locking pin 434 from either the first slot 440 or the second slot 436, thus unlocking the connector shaft 426. In the compressed/pushed in position, the pull-back spring 430 extends the locking pin 434 into either the first slot 440 or the second slot 436, thus locking the connector shaft 426. When the locking pin 434 has been removed from either the first slot 440 or the second slot 436, the spring back mechanism 454 generally actuates the push-back spring 428 of the primary solenoid 422, e.g., a push-type solenoid, into a compressed or expanded position. In particular, in the expanded/pushed back position, the push-back spring 428 pulls the connector shaft 426 such that the spring-loaded clamp actuation mechanism 460 causes the clamp 462, i.e., the first and second clamp members, to clamp around an organ. In the compressed/pushed in position, the push-back spring 428 pushes the connector shaft 426 such that the spring-loaded clamp actuation mechanism 460 causes the clamp 462 to open and/or release the organ. In other exemplary embodiments, it should be understood that the connector shaft 426 may include, e.g., two, three, four, five, six, and the like, slots for the locking pin 434 which permit the first and second clamp members 208a and 208b to be actuated in a plurality of clamping positions relative to each other in order to adjust the clamping pressure on the organ.

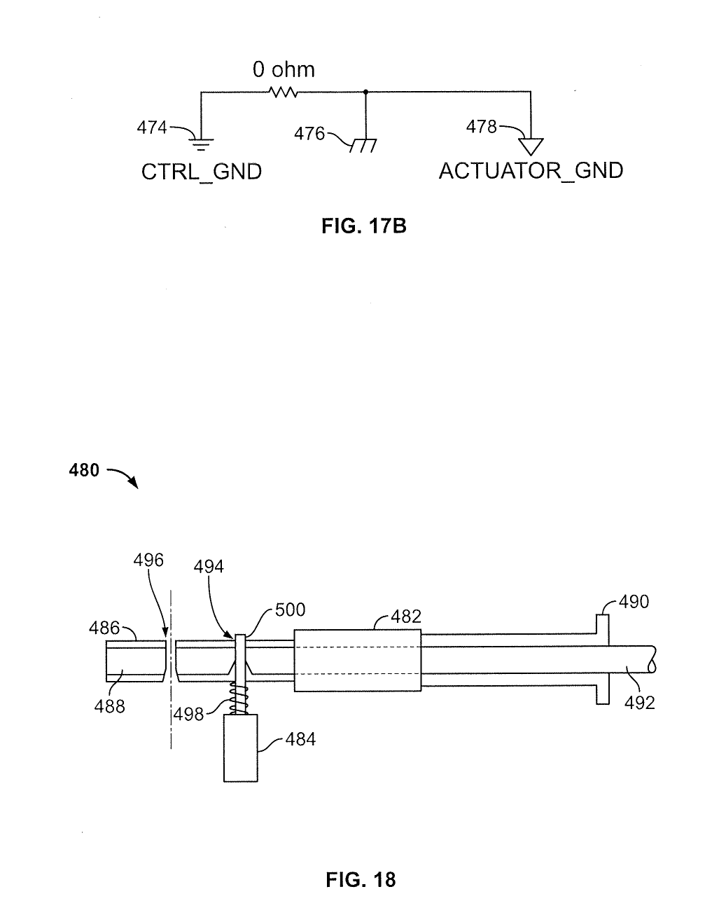

FIGS. 17A and B illustrate electrical circuit diagrams for the single-pole solenoid actuation mechanism 420. In particular, FIG. 17A shows a control bit 464, a switch 466, a solenoid pole 468, a flyback diode 470, and an actuator voltage 472. The control bit 464 generally controls the single-pole solenoid actuation mechanism 420 through, e.g., a transistor. As would be understood by those of ordinary skill in the art, the control bit 464 generally actuates the switch 466 which in turn actuates the solenoid pole 468, i.e., actuates the single-pole solenoid actuation mechanism 420 to regulate the clamping pressure of the first and second clamp members 208a and 208b. control circuitry is generally isolated from the actuator circuit through an opto-isolator. In addition, flyback voltage protection can generally be provided by the flyback diode 470. FIG. 17B illustrates the electrical circuit diagram for the control ground 474, the earth ground 476, and the actuator ground 478.

Turning now to FIG. 18, a double-pole solenoid actuation mechanism 480 is provided, generally including a primary solenoid 482 and a secondary solenoid 484. The primary solenoid 482 is generally connected to the connector shaft 492, which in turn is mechanically connected to the clamping mechanism 212. In general, the connector shaft 492 translates inside a bushing 490 to maintain a substantially planar translation. Thus, translation of the connector shaft 492 regulates the clamping pressure applied on an organ by the first and second clamp members 208a and 208b. An inner shaft 488 connected to the primary solenoid 482 generally translates within an outer sleeve housing 486 and includes a first slot 494, i.e., an unclamped position, and a second slot 496, i.e., a clamped position, which are configured and dimensioned to receive a locking pin 500 associated with the secondary solenoid 484. In particular, a spring 498 of the secondary solenoid 484 may be actuated, e.g., compressed, expanded, and the like, to insert and/or remove the locking pin 500 from either the first or second slot 494 or 496. As would be understood by those of ordinary skill in the art, removing the locking pin 500 from either the first or second slot 494 or 496 generally allows translation of the primary solenoid 482 such that the first and second clamp members 208a and 208b are clamped or unclamped around an organ. Once the primary solenoid 482 has been actuated sufficiently to create the desired clamping pressure around an organ, the spring 498 is generally actuated to insert the locking pin 500 into the appropriate slot to maintain the clamping pressure around the organ. Although two slots are shown in FIG. 18, in other exemplary embodiments, the inner shaft 488 can include, e.g., two, three, four, five, and the like, slots for the locking pin 500 which permit the first and second clamp members 208a and 208b to be actuated in a plurality of clamping positions relative to each other in order to adjust the clamping pressure on the organ.

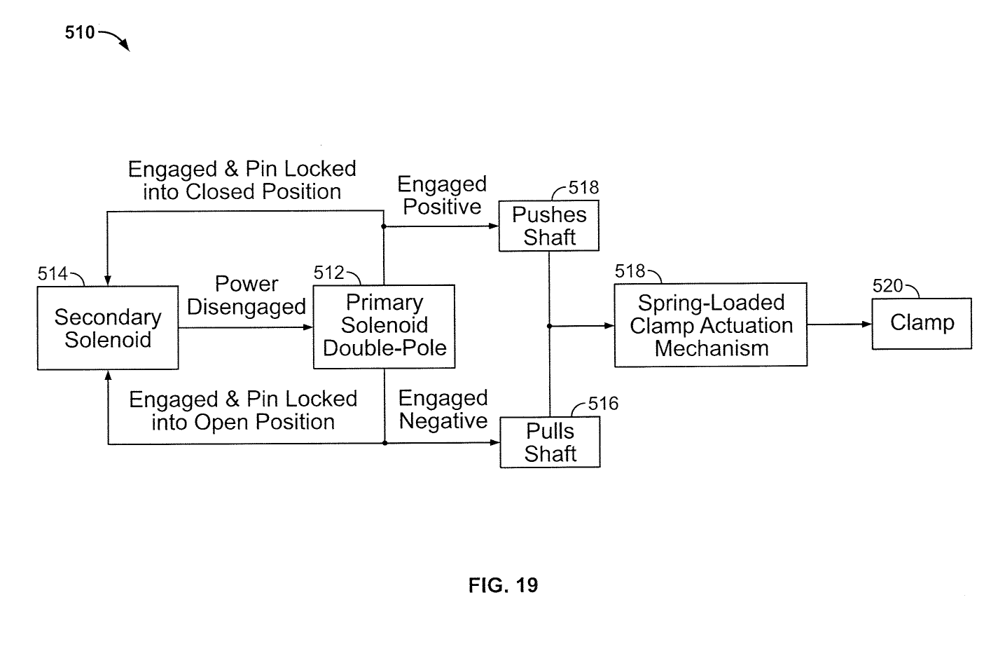

With reference to FIG. 19, a flow chart 510 of a double-pole solenoid actuation mechanism 480 is provided. In particular, the primary solenoid 512, i.e., a double-pole primary solenoid, is generally configured for active engagement for push or pull. The primary solenoid 512 may further include a power cut-off in the push and/or pull positions with the secondary solenoid 514 locking pin 500 for safety in case of accidental power cut-off. The secondary solenoid 514 can normally be in an open position via the spring 498. Actuation of the secondary solenoid 514 generally enables the inner shaft 488 to translate into the desired position, e.g., regulate the clamping pressure between the first and second clamp members 208a and 208b. As can be seen from FIG. 19, the primary solenoid 512 may be negatively or positively engaged. Negative engagement of the primary solenoid 512 generally pulls the connector shaft 492 (516). Pulling the connector shaft 492 generally actuates the spring-loaded clamp mechanism 518, i.e., the clamping mechanism 212, to reduce the clamping pressure around the clamp 520, i.e., the first and second clamp members 208a and 208b. Once the first and second clamp members 208a and 208b have been actuated into an open position, the spring 498 of the secondary solenoid 514 may be actuated to insert the locking pill 500 into the appropriate slot to lock the clamping forceps into the open position. Positive engagement of the primary solenoid 512 generally pushes the connector shaft 492. Pushing the connector shaft 492 generally actuates the spring-loaded clamp mechanism 518, i.e., the clamping mechanism 212, to increase the clamping pressure around the clamp 520, i.e., the first and second clamp members 208a and 208b. Once the first and second clamp members 208a and 208b have been actuated into a position with the desired clamping pressure, the spring 498 of the secondary solenoid 514 may be actuated to insert the locking pin 500 into the appropriate slot to lock the clamping forceps in the clamped and/or closed position. The secondary solenoid 514 may further disengage the power to the primary solenoid 512. As would be understood by those of ordinary skill in the art, the single and double-pole solenoid mechanisms discussed herein may be implemented for, e.g., regulating the clamping pressure of the first and second clamp members 208a and 208b, actuating the first and second clamp members 208a and 208b between a folded and unfolded position, and the like.

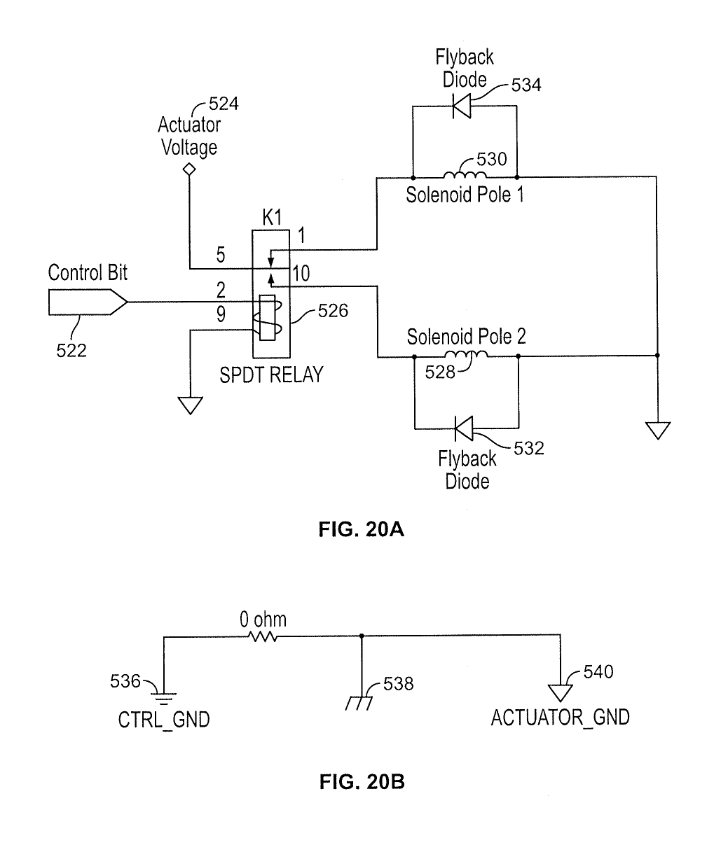

FIGS. 20A and B illustrate electrical circuit diagrams for the double-pole solenoid actuation mechanism 480. In particular, FIG. 20A shows a control bit 522, an actuator voltage 524, a switch 526, a first solenoid pole 530, a first flyback diode 534, a second solenoid pole 528, and a second flyback diode 532. The control bit 522 generally controls the double-pole solenoid actuation mechanism 480 through, e.g., a single pole double throw relay. As would be understood by those of ordinary skill in the art, the control bit 522 generally actuates the switch 526 which in turn actuates the first and/or second solenoid poles 530 and 528, i.e., actuate the double-pole actuation mechanism 480 to regulate the clamping pressure of the first and second clamp members 208a and 208b. Control circuitry is generally isolated from the actuator circuit by the SPOT relay, i.e., switch 526. Flyback voltage protection can generally be provided by the first and second flyback diodes 534 and 532. FIG. 20B illustrates the electrical circuit diagram for the control ground 536, the earth ground 538, and the actuator ground 540.

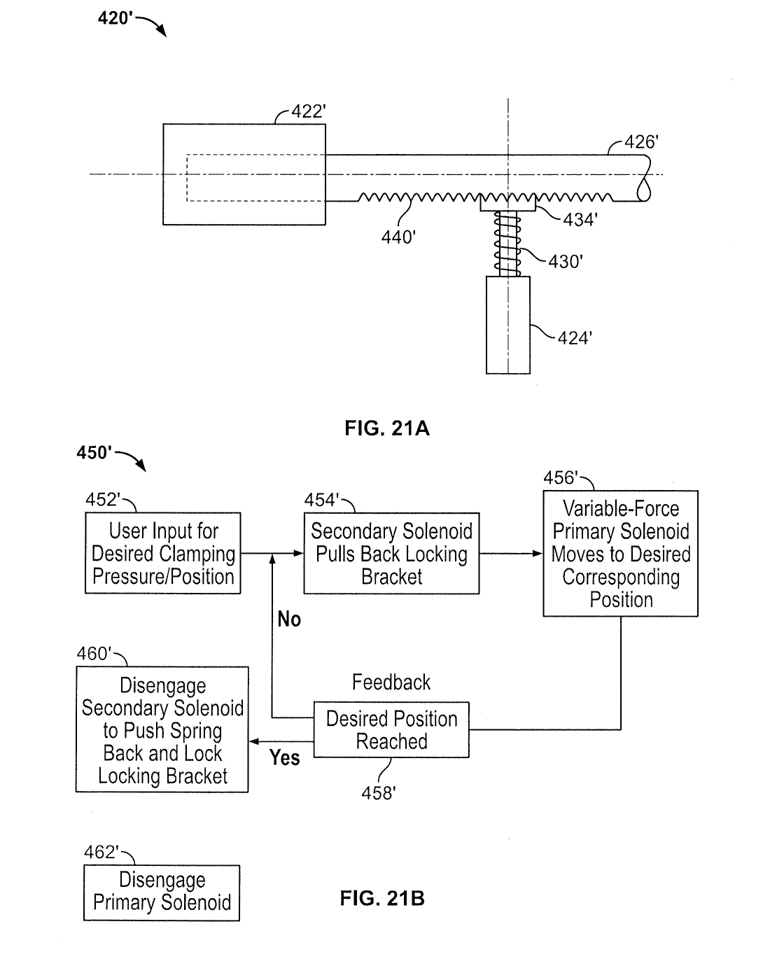

An exemplary variable position solenoid actuation mechanism 420′ is illustrated in FIG. 21A for a clamping forceps according to the present disclosure. It should be understood that the variable position solenoid actuation mechanism 420′ may be implemented with, e.g., a single-pole solenoid, a double-pole solenoid, and the like. The variable position solenoid actuation mechanism 420′ generally includes a primary solenoid 42′, e.g., a variable force solenoid, and the like, and a secondary solenoid 424′, e.g., a solenoid for locking a bracket, a push-type solenoid, and the like. The connector shaft 426′, e.g., a movable shaft, a track, a rack, and the like, generally extends from the variable position solenoid actuation mechanism 420′ and is in mechanical communication with the first and second clamp members 208a and 208b. The connector shaft 426′ generally includes a locking surface 440′ defined by a textured surface, e.g., grooves, ridges, teeth, and the like. The variable position solenoid actuation mechanism 420′ generally further includes a push-back spring 430′. A textured surface, e.g., grooves, ridges, teeth, and the like, of the locking bracket 434′ of the variable position solenoid actuation mechanism 420′ can generally be positioned such that the locking bracket 434′ mates with the locking surface 440′ of the connector shaft 426′. As would be understood by those of ordinary skill in the art, the variable position actuation mechanism 420 can be actuated to pull back the secondary solenoid 424′ to allow translation of the shaft 426′, and can further be actuated to push the secondary solenoid 424′ such that the locking bracket 434′ mates with the locking surface 440′ at the desired position. This actuation in turn generally regulates the clamping force of the first and second clamp members 208a and 208b around an organ or other structure. The plurality of, e.g., ridges, teeth, and the like, of the locking surface 440′ on the shaft 426′ further permit variable clamping positions to be utilized with respect to the first and second clamp members 208a and 208b. As would be understood by those of ordinary skill in the art, the sizes of the textured surface features on the locking surface 440′ may be varied to allow fine and/or gross adjustment of the clamping position of the first and second clamp members 208a and 208b.

With reference to FIG. 21B, a flow chart 450′ of a variable position solenoid actuation mechanism 420′ is provided. In particular, the user generally inputs the desired clamping pressure position (452′) at, e.g., a user interface, and the like. The secondary solenoid 424′ is generally actuated to pull back the locking bracket 434′ to release the locking bracket 434′ from the locking surface 440′ of the shaft 426′ (454′). The variable-force primary solenoid 422′ can be actuated to move to a desired corresponding position, i.e., a position which provides the desired clamping pressure between the first and second clamp members 208a and 208b input by the user (456′). A feedback signal, e.g., a visual feedback, an audio feedback, a position feedback, and the like, can generally be generated to indicate whether the desired clamping pressure and/or clamping position has been reached (458′). As can be seen in FIG. 21B, if the desired clamping pressure has not been reached, steps 454′, 456′ and 458′ may be repeated until the desired clamping pressure has been achieved. If a desired clamping pressure has been reached, the secondary solenoid 424′ can generally be disengaged to push the push-back spring 430′ back, thereby translating the locking bracket 434′ against the locking surface 440′ of the shaft 426′ (460′). Once the locking bracket 434′ has locked the shaft 426′ in the position corresponding to the desired clamping pressure, the primary solenoid 422′ can be disengaged (462′). As would be understood by those of ordinary skill in the art, to reduce the clamping pressure of the first and second clamp members 208a and 208b, a similar process may be implemented to translate the shaft 426′ into the appropriate interlocked position with the locking bracket 424′.

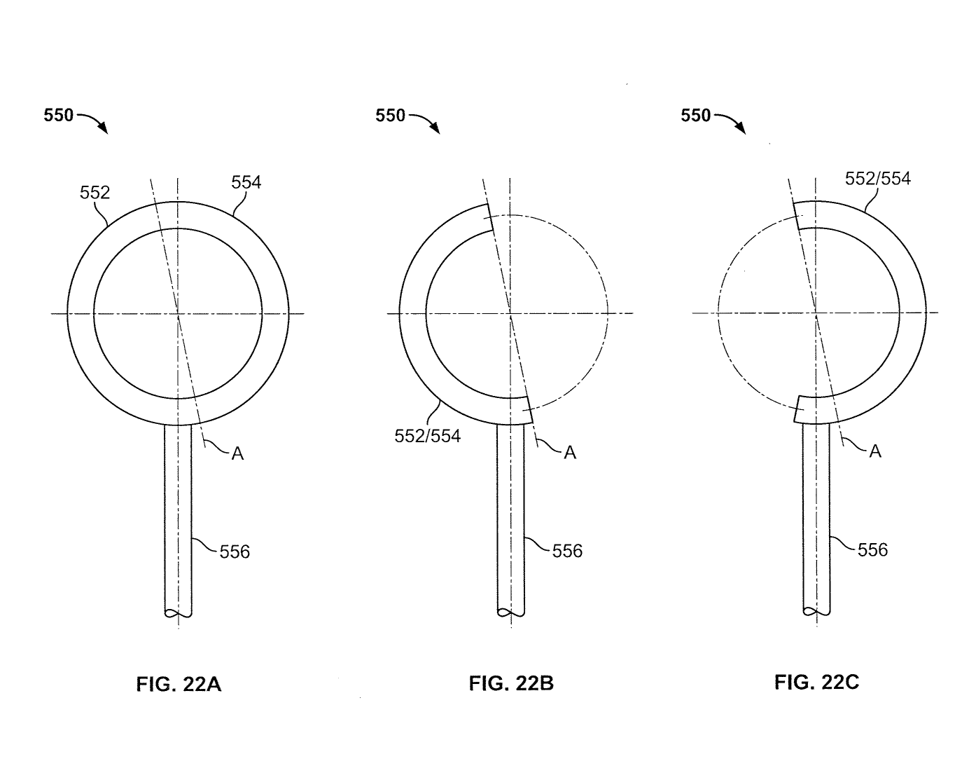

In accordance with embodiments of the present disclosure, an exemplary clamping forceps 550 is provided in FIGS. 22A-C for a transient ischemia design. As discussed herein, it should be understood that a transient ischemia design generally refers to first and second clamp members structured so as to define a variable perimeter extent that permits variability and/or adjustment in the degree to which a tumor or other structure is encircled by the first and second clamp members. For example, the first and second clamp members generally include clamp sides, i.e., leaves, configured and dimensioned to fold at, e.g., 30°, 60°, 180°, and the like, increments. FIGS. 22A-C illustrate only a first clamp member of the clamping forceps 550. However, it should be understood that the clamping forceps 550 include substantially similar first and second clamp members.

With respect to FIG. 22A, the clamp members of the exemplary clamping forceps 550 generally include a first clamp side 552 and a second clamp side 554, i.e., first and second leaves, mechanically connected to a shaft 556, e.g., a clamping mechanism shaft, and the like. The shaft 556 mechanically connects the clamp members to the elongated body section and/or the handle section (not shown) of the clamping forceps 550. The clamp members are generally configured and dimensioned to permit the first clamp side 552 to flip along a variable axis A onto the second clamp side 554, and vice versa. In particular, the first and second clamp sides 552 and 554 are generally diametrically opposed relative to each other at an initial angle of 0°. With specific reference to FIG. 22B, the second clamp side 554 has been flipped along the variable axis A onto the first clamp side 552, thereby creating a substantially C-shaped clamping member. With specific reference to FIG. 22C, the first clamp side 552 has been flipped along the variable axis A onto the second claim side 554, thereby creating a substantially C-shaped clamping member in an opposing direction to FIG. 22B. In other exemplary embodiments, the clamping member may be a C-shaped clamp member without the capability of expanding to a full-perimeter clamp member and with the ability to flip along the variable axis A to change the orientation of the C-shaped clamp member. As would be understood by those of ordinary skill in the art, the first and second clamp members may be flipped in conjunction with each other and/or independently of each other. For example, a first clamp member may be flipped into a C-shaped clamp member, while the second clamp member may remain a full-perimeter clamp member. As a further example, both the first and second clamp members may be flipped into C-shaped clamp members. Thus, a user may regulate the areas of the organ and/or surgical site which are clamped to prevent blood flow passage, while permit other areas of the organ and/or surgical site to receive blood flow to reduce the surgical margin.

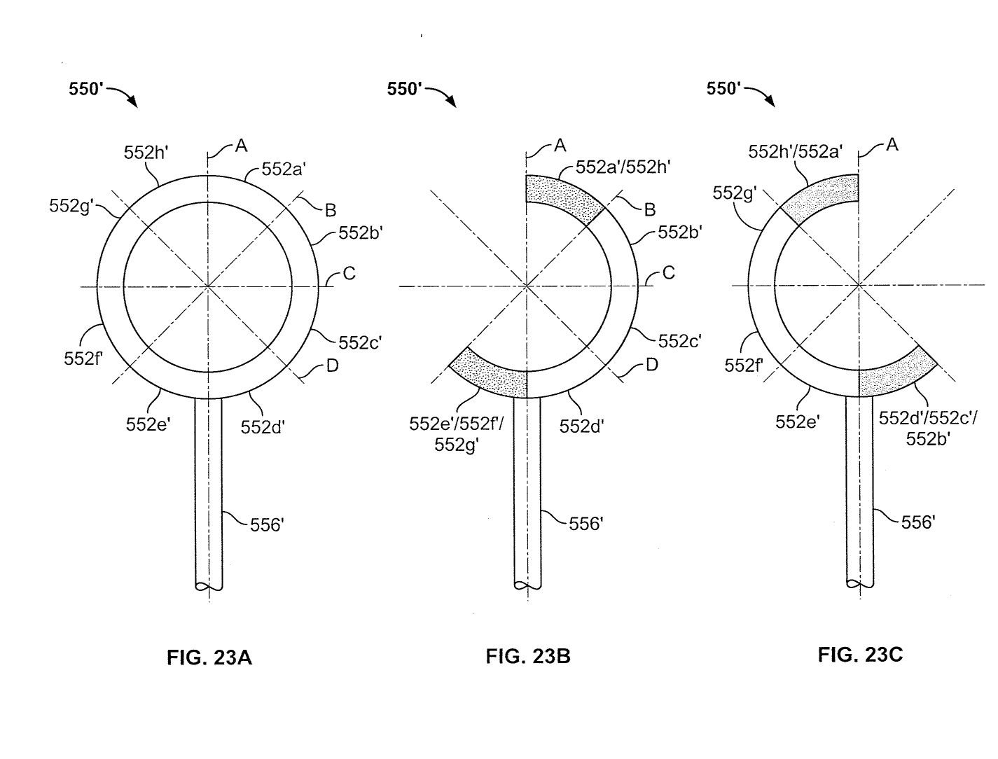

FIGS. 23A-C illustrate an alternative exemplary embodiment of the transient ischemia clamping forceps 550′. As discussed herein, it should be understood that a transient ischemia design generally refers to first and second clamp members structured so as to define a variable perimeter extent that permits variability and/or adjustment in the degree to which a tumor or other structure is encircled by the first and second clamp members. In particular, rather than including a first and second clamp side 552 and 554 connected to a connector shaft 556 which are capable of folding and/or flipping, the exemplary clamping forceps 550′ generally includes first, second, third, fourth, fifth, sixth, seventh, and eighth clamp sides 552a′-552h′ (hereinafter collectively “clamp sides 552a′-552h′”) which are capable of folding and/or flipping onto each other along axes A, B, C and D, respectively. Although illustrated with eight clamp sides 552a′-552h′, in other exemplary embodiments, the clamping forceps 550′ can include, e.g., two, three, four, five, six, seven, eight, nine, ten, eleven, twelve, and the like, clamp sides. For example, the clamp sides may be divided by 15°, 30°, 45°, 60°, and the like, intervals around the perimeter of the first and second clamp members.

With specific reference to FIG. 23B, the eighth clamp side 552h′ has been actuated to flip and/or fold onto the first clamp side 552a′ and the sixth and seventh clamp sides 552f and 552g′ have been actuated to flip and/or fold onto the fifth clamp side 552e′. Similarly, in FIG. 23C, the first clamp side 552a′ has been actuated to flip and/or fold onto the eighth clamp side 552h′ and the second and third clamp sides 552b′ and 552c′ have been actuated to flip and/or fold onto the fourth clamp side 552d′. As would be understood by those of ordinary skill in the art, the capability of flipping and/or folding the clamp sides onto each other to vary the perimeter extent of the first and second clamp members allows the user to vary the areas of the surgical site into which blood flow is permitted or stopped. This flexibility generally decreases the surgical margin with respect to the healthy tissue surrounding a tumor by permitting the healthy tissue to receive the blood flow necessary to prevent and/or reduce permanent damage.

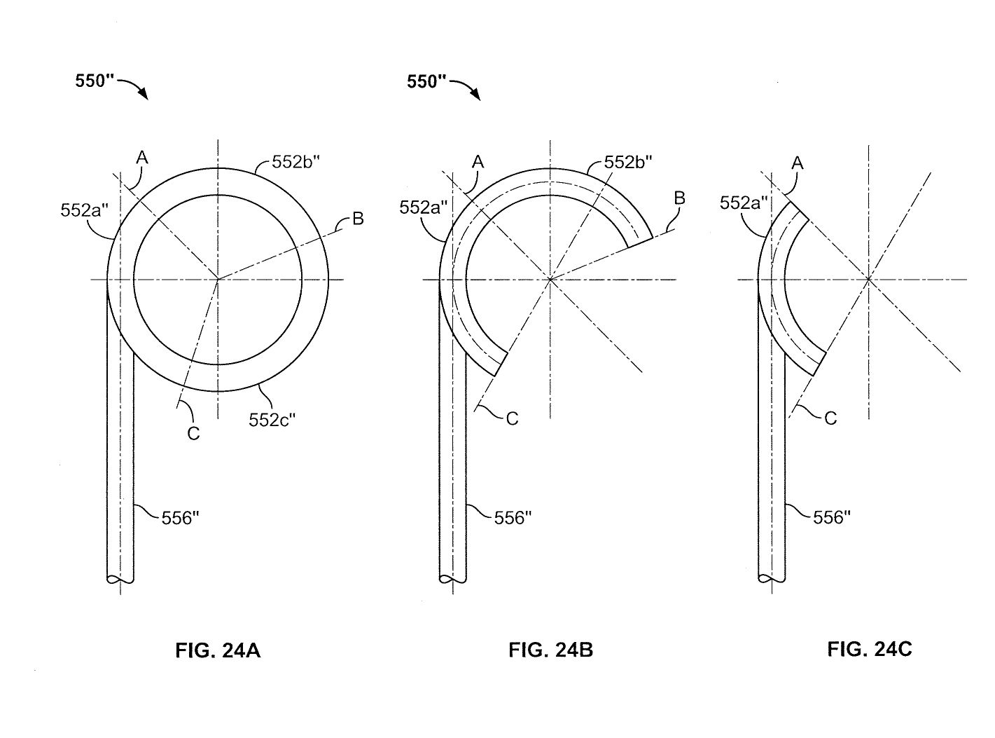

Turning now to FIGS. 24A-C, an alternative exemplary embodiment of transient ischemia clamping forceps 550″ is provided. As discussed herein, it should be understood that a transient ischemia design generally refers to first and second clamp members structured so as to define a variable perimeter extent that permits variability and/or adjustment in the degree to which a tumor or other structure is encircled by the first and second clamp members. Rather than symmetrically connecting to the first and second clamp members at a central location, the exemplary clamping forceps 550″ generally include a connector shaft 556″ mechanically connected to the first and second clamp members at an offset location. The first and second clamp members generally include first, second and third clamp sides 552a″, 552b″ and 552c″, respectively, which are configured and dimensioned to flip and/or fold at axes A, B and C. In particular, axes A, B and C may be positioned at approximately 120° relative to each other. As can be seen in FIG. 24B, the third clamp side 552c″ has been actuated to flip and/or fold onto one of the first or second clamp sides 552a″ and 552b. In FIG. 24C, the second and third clamp sides 552b″ and 552c″ have been actuated to flip and/or fold onto the first clamp side 552a″. Thus, in a full-perimeter position, the first, second and third clamp sides 552a″, 552b″ and 552c″ remain unfolded and/or unflipped, thus clamping 360° of the organ around the perimeter. If one clamp side is actuated to flip and/or fold, the first and second clamp members generally provide clamping pressure at approximately 240° around the perimeter. Further, if two clamp sides are actuated to flip and/or fold, the first and second clamp members generally provide clamping pressure at approximately 120° around the perimeter. Similar to the exemplary clamping forceps 550 and 550′ discussed above, the clamping forceps 550″ permit the user to vary and/or adjust the perimeter extent of the first and second clamp members to vary and/or adjust the degree to which a tumor or other structure is encircled by the first and second clamp members. Thus, blood flow may be maintained to desired portions of tissue surrounding a tumor, while other portions of tissue surround a tumor may be clamped to prevent blood flow.

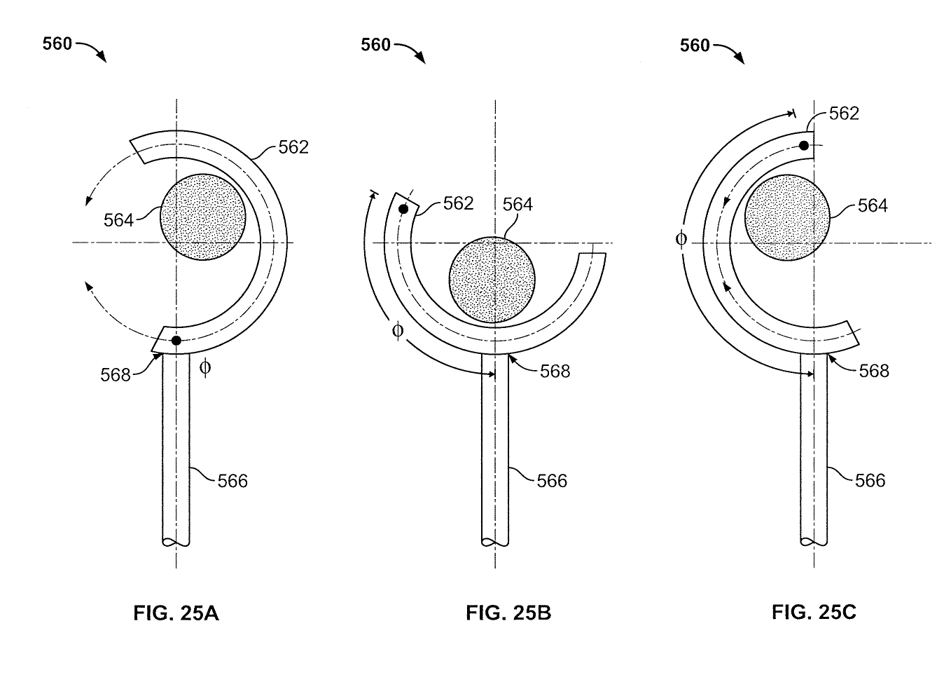

In accordance with embodiments of the present disclosure, an exemplary rotating clamping forceps 560 is provided in FIGS. 25A-C. In particular, the clamping forceps 560 may be implemented in conjunction with and/or separate from the folding and/or flipping embodiments of the first and second clamp members discussed above. The exemplary clamping forceps 560 generally includes a clamp member 562 configured and dimensioned to at least partially encircle a tumor 564 or other structure. Although illustrated as a substantially C-shaped clamp member, in other exemplary embodiments, the clamp member may be defined by, e.g., a 360° perimeter, a variable and/or folding perimeter, and the like. In addition, although illustrated as one clamp member 562, it should be understood that the clamping forceps 560 includes substantially similar first and second clamp members as discussed above. The clamp member 562 is generally in mechanical communication with a connector shaft 566. Further, the clamp member 562 is generally configured to axially rotate about a distal point 568 of the connector shaft 566 at an angle θ relative to the connector shaft 566 axis.

For example, FIG. 25A illustrates the clamp member 562 at an approximately 0° rotation position, FIG. 25B illustrates the clamp member at an approximately 119° rotation position, and FIG. 25C illustrates the clamp member at an approximately 175° rotation position relative to the connector shaft 566. It should be understood that the clamp member 562 may be rotated in the range of between 0° to 360° relative to the connector shaft 566 axis. The axial rotation of the clamp member 562 may be regulated by a user at a user interface, e.g., a switch or dial located at the handle section of the clamping forceps, and the like. The axial rotation of the clamp member 562 may be actuated by, e.g., a regulated rotation of the connector shaft 566, and the like. Thus, the clamp member 562 may be rotated intermittently intra-operatively to modulate the warm ischemia time associated with the tissue in the tumor excision area within the perimeter of the clamp member 562. For example, once a surgeon has completed the surgical procedure at one side of the tumor 564, the clamp member 562 may be rotated to allow the passage of blood flow to the tissue surrounding the tumor 564 in order to reduce the surgical margin.

With reference to FIG. 26, a toggle switch system 570 for rotating the first and second clamp members located at, e.g., the handle section 574 of a clamping forceps, is illustrated. In some exemplary embodiments, the toggle switch system 570 may be implemented as a digital read-out. In particular, the handle section 574 generally includes a shaft and/or an elongated body section 572 in mechanical communication with the connector shaft 566 of the clamping forceps 560. The handle section 574 may further include a toggle switch 576 for actuating and/or regulating, e.g., the angle of rotation of the first and second clamp members, a duty-cycle for a variation in clamping force, and the like. In some exemplary embodiments, the handle section 574 generally includes a display 578 which provides, e.g., the duration of warm ischemia time in the clamped position, a visual of a duty cycle adjustment, the angle of rotation, and the like, in real-time to the user. Although illustrated as a component of the handle section 574, it should be understood that the toggle switch 576 and/or the display 578 may be a separate and/or stand-alone component relative to the clamping forceps 560. In some exemplary embodiments, the handle section 574 generally permits, e.g., rotation of the connector shaft 566, rotation of the elongated body section 572, rotation of a shaft within the elongated body section 572, rotation of the handle section 574 relative to the elongated body section 572, and the like. For example, the exemplary handle section 574 may permit the surgeon to position and clamp the clamping, forceps as desired and further to angularly rotate the handle section 574 relative to the elongated body section 572 in order to create a larger operating space around the patient. In some exemplary embodiments, the handle section 574 may be detached from the elongated body section 572, while maintaining the clamping pressure around the organ. The exemplary handle section 574 may also be utilized in accordance with the embodiments of clamping forceps discussed herein, including, for example, the embodiments of FIGS. 14-25.

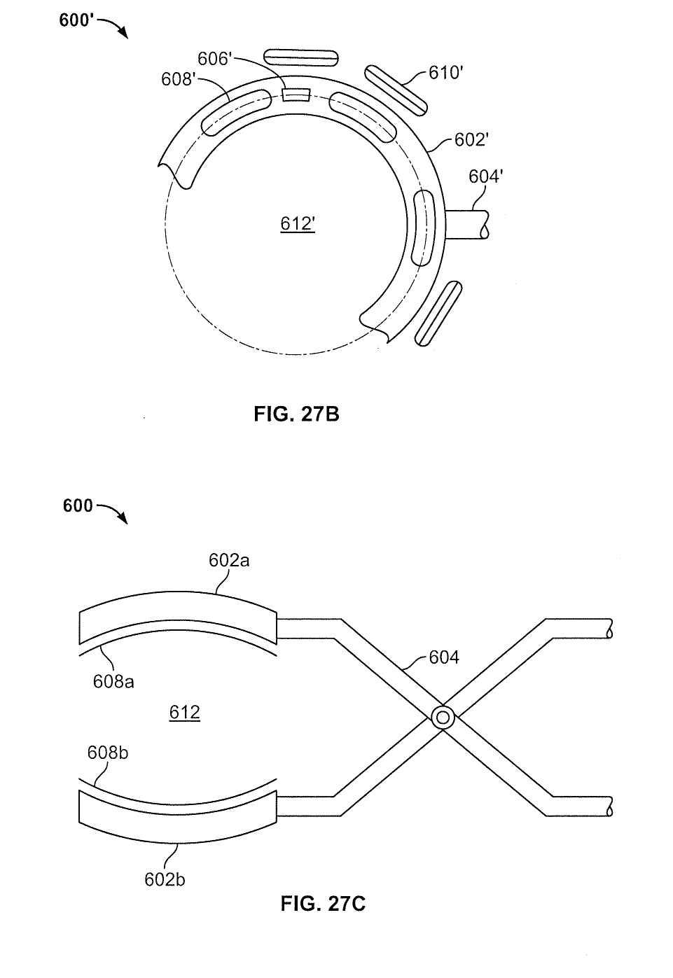

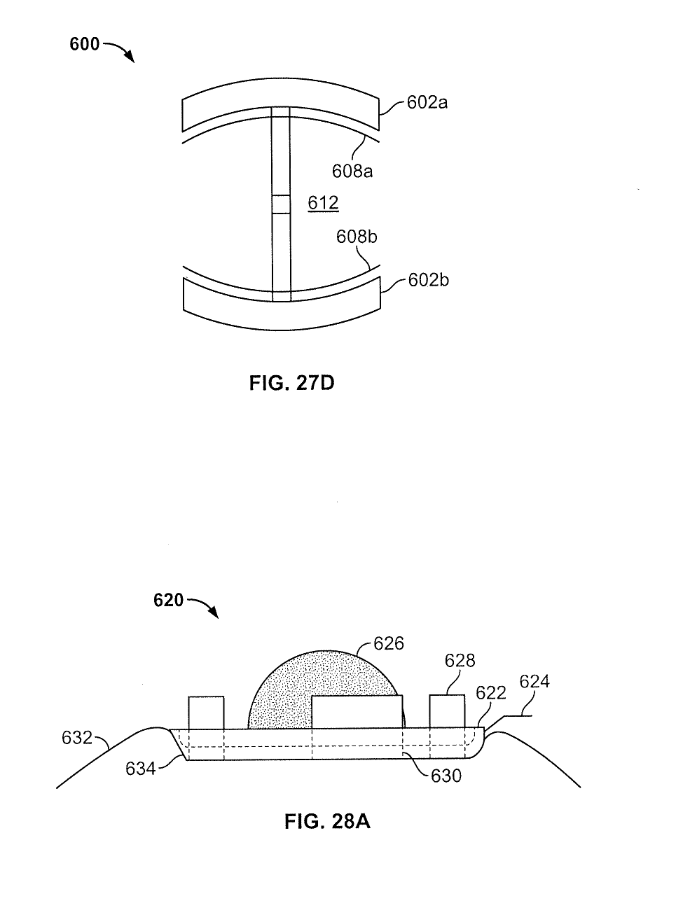

In accordance with embodiments of the present disclosure, an exemplary clamping forceps 600 is provided in FIG. 27A. The exemplary clamping forceps 600 generally includes means for fixating at least one of the first clamp member and the second clamp member relative to a tissue. The means for fixating at least one of the first clamp member and the second clamp member 602 relative to the tissue generally includes at least one of, e.g., a suction mechanism, a textured surface, a coated surface, and the like, deployed on a clamping surface. Although not illustrated, an exemplary textured surface may include, e.g., one or more spikes, ridges, and the like, to penetrate and/or hold on to tissue during a surgical procedure. The spike(s) may be, e.g., ⅛ mm, ¼ mm, and the like, in length. In addition, an exemplary coated surface can include, e.g., an adhesive and/or sticky coating, a hydrophilic coating, a hydrophobic coating, a therapeutic coating, and the like. It should be understood that in some exemplary embodiments, the suction mechanism, textured surface, coated surface, and the like, may be implemented in combination and/or separately on the first and second clamp members 602. For example, the suction mechanism may be implemented to remove, e.g., tissue, liquified, material, and the like, from the surgical site. Although illustrating one clamp member 602, it should be understood that the exemplary clamping forceps 600 includes two clamp members 602 which are typically substantially similar in structure and/or function. The clamping forceps 600 generally includes two clamp members 602 and a clamping mechanism 604 which is generally movably mounted (at least in part) with respect to an elongated body section (not shown). At least one of the first and second clamp members 602 further generally includes at least one sensor 606, as discussed in greater detail above.

The suction mechanism of the exemplary clamping forceps 600 of FIG. 27A can generally be, e.g., a suction gasket 608, and the like. The suction gasket 608 generally facilitates a tight seal of the clamping forceps 600 onto the surface of the tissue. In some exemplary embodiments, the suction gasket 608 may be fabricated from a malleable, biocompatible polymeric material. Further, the suction gasket 608 generally provides a softer material than the clamp member 602 to reduce the risk of damage to the soft organ upon clamping. The suction gasket 608 generally defines a gasket around the inner and outer perimeter of the clamp member 602 which is in communication with a source of negative pressure flow and in communication with a source of positive pressure flow, e.g., at least one slot. For example, the clamp member 602, the clamping mechanism 604, the elongated body section, and the like, generally include at least one conduit in communication with the suction gasket 608. In some exemplary embodiments, the conduit may pass, e.g., inside the clamp member 602, inside the clamping mechanism 604, inside the elongated body section, and the like. In other exemplary embodiments, the conduit may pass, e.g., outside the clamp member 602, outside the clamping mechanism 604, outside the elongated body section, and the like.

Delivery of a negative pressure flow to the suction gasket 608 is generally effective to draw tissue 612 into the suction gasket 608. Delivery of a positive pressure flow to the suction gasket 608 is generally effective to push out the drawn tissue 612 from the suction gasket 608. As would be understood by those of ordinary skill in the art, once a user has positioned the first and second clamp members 602 around an organ or other structure in the desired position, the suction mechanism, e.g., the suction gasket 608, can be actuated to draw in tissue 612 into the suction gasket 608 in order to fixate the clamping forceps 600 during the surgical procedure. Fixation of the clamping forceps 600 may, e.g., ensure that the clamping forceps 600 do not slip and/or move during the surgical procedure, ensure a fixation of the clamping forceps 600 in a desired clamping area prior to clamping around an organ, and the like. Once the surgical procedure has been completed and/or when the user desires to reposition the clamping forceps, the tissue 612 may be drawn or pushed out of the suction gasket 608 with a positive pressure flow and the clamping forceps 600 may be repositioned, as desired. Further, the clamping forceps 600 may be implemented in conjunction with surgical suture clips 610 for tying off sutures utilized in the surgical procedure, as described in PCT International Application No. PCT/US2011/066575 entitled “Sliding Overhead Clip and Associated Methods”, the entire content of which is incorporated herein by reference. For example, the surgical suture clips 610 may be positioned around the periphery of the clamping forceps 600 as illustrated in FIG. 27A to tie off the sutures implemented in the surgical procedure. In some exemplary embodiments, the clamping forceps 600 can also be implemented with and/or be configured to accommodate a variety of surgical suture clips 610, e.g., Lapra-Ty®, Hem-O-Lok®, and the like, suture clips.

With reference to FIG. 27B, an exemplary clamping forceps 600′ is illustrated with an alternative exemplary suction mechanism. In particular, the suction mechanism of the clamping forceps 600′ can be, e.g., at least one spaced opening 608′. The at least one spaced opening 608′ may be, e.g., a vacuum slot, an air slot, and the like, positioned around the periphery of the first and second clamp members 602′. The exemplary clamping forceps 600′ is generally substantially similar in structure and/or function to the clamping forceps 600 described above, including, e.g., a first and second clamp member 602′, a clamping mechanism 604′, a sensor 606′, implementation with suture clips 610′, and the like. Although illustrated with a plurality of spaced openings 608′, in other exemplary embodiments, the clamping forceps 600′ may include, e.g., one, two, three, four, five, six, seven, eight, and the like, spaced openings 608′. The suction mechanism generally includes at least one of the first and second clamp members 602′ defining at least one spaced opening 608′ in communication with a source of negative pressure flow and in communication with a source of positive pressure flow. The first and second clamp members 602′, the clamping mechanism 604′, the elongated body section, and the like, generally include at least one conduit in communication with the at least one spaced opening. The at least one conduit is generally in communication with a source of negative and/or positive pressure flow. Delivery of the negative pressure flow to the at least one spaced opening 608′ is generally effective to draw tissue 612′ into the at least one spaced opening 608′. Delivery of a positive pressure flow to the at least one spaced opening 608′ is generally effective to push out the drawn tissue 612′ from the at least one spaced opening 608′. Thus, similar to the clamping forceps 600, the clamping forceps 600′ may be positioned and fixated onto the tissue 612′ in the vicinity of the surgical area in order to ensure a resilient fixation during the surgical procedure. In some exemplary embodiments, the spaced openings 608′ may be implemented for introduction of a therapeutic treatment, e.g., a tissue excision, a hemostatic treatment, an RF therapy, a thermal treatment, a cryogenic treatment, a brachytherapy, a radiation therapy, a therapeutic agent, a pharmaceutical agent, a genomic agent, and the like, to the tissue 612′.

FIGS. 27C and D illustrate the side and detailed views of the exemplary clamping forceps 600, generally including a first clamp member 602a and a second clamp member 602b. Each of the first and second clamp members 602a and 602b generally includes a first and second suction gasket 608a and 608b, respectively. In some exemplary embodiments, the first and second clamp members 602a and 602b may be implemented without suction gaskets 608a and 608b. As illustrated, the first and second clamp members 602a and 602b have been positioned around tissue 612 of an organ. The first and second clamp members 602a and 602b and/or the first and second suction gaskets 608a and 608b can be, e.g., configured to substantially match the topographical contour of an organ surface, and the like. The topographically contoured surface generally ensures a stronger, more accurate, and/or uniform clamping action/force distribution around the tumor to be excised. A negative pressure flow may be utilized in conjunction with the first and second suction gaskets 608a and 608b to draw in the tissue 612 and thereby fixate the first and second clamp members 602a and 602b relative to the tissue 612 during a surgical procedure.

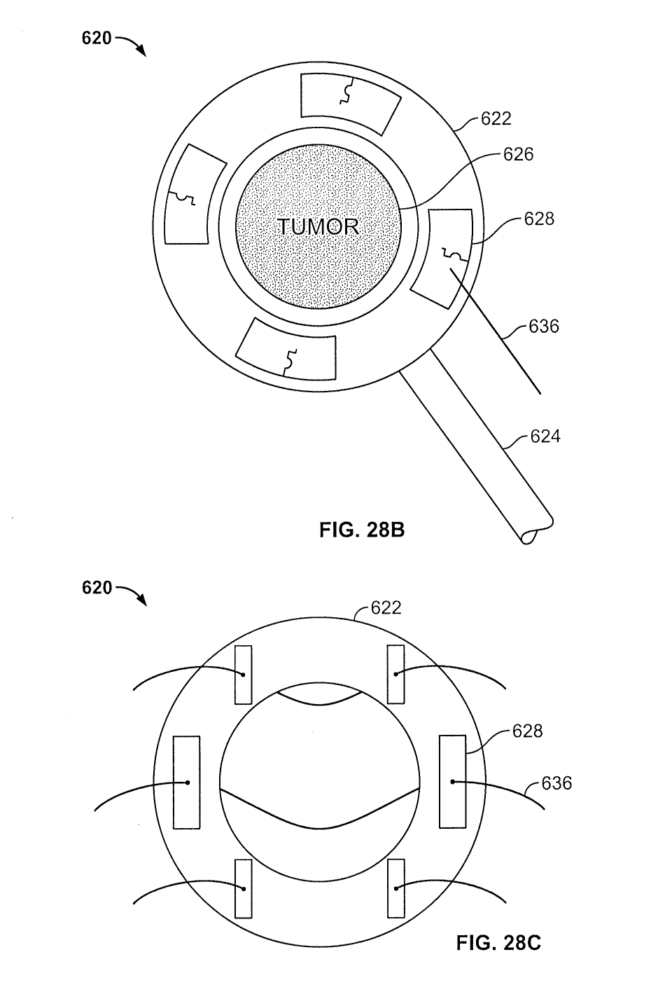

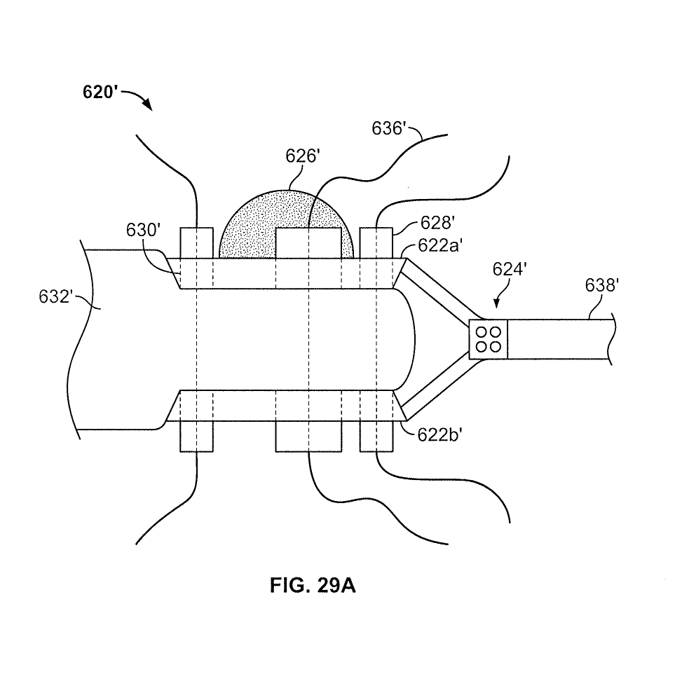

Turning to FIGS. 28A-C, an exemplary embodiment of a clamping forceps 620 is provided for implementation with surgical suture clips 628 for tying off sutures 636 utilized in a surgical procedure for removal of a tumor 626, as described in PCT International Application No. PCT/US2011/066575 entitled “Sliding Overhead Clip and Associated Methods”, the entire content of which was previously incorporated herein by reference. In some exemplary embodiments, the clamping forceps 600 can also be implemented with and/or be configured to accommodate a variety of surgical suture clips 628, e.g., Lapra-Ty®, Hem-O-Lok®, and the like, suture clips. In particular, the clamping forceps 620 generally includes first and second clamp members 622, a clamping mechanism 624, and an elongated body section (not shown). It should be understood that FIGS. 28A-C only illustrate a first clamp member 622 on one side of an organ 632 and that a second clamp member substantially similar in function and structure is generally positioned on the opposite side of the organ 632. The first clamp member 622 generally includes a curved edge 634 for depressing tissue of the organ 632. The first clamp member 622 generally further includes at least one void 630 configured and dimensioned to receive a surgical suture clip 628. In some exemplary embodiments, Surgicel® hemostatic bolster, for example, may be inserted into the ring area within the first clamp member 622 perimeter, i.e., on the tumor bed. Thus, once a surgical suture clip 628 has been utilized to properly position a suture 636, the first clamp member 622 may be unclamped from the organ 632, i.e., the first clamp member 622 may be lifted off from the organ 632 while permitting the surgical suture clip 628 to remain fixed to the organ 632 and keeping the Surgicel® in place. The suture 636 and corresponding suture clips 628 can then be tightened in the normal fashion, thereby generally minimizing blood loss during the procedure. Although illustrated as including a plurality of voids 630 and implementing a plurality of surgical suture clips 628, in other exemplary embodiments, more or less voids 630 and/or surgical suture clips 628 may be implemented.

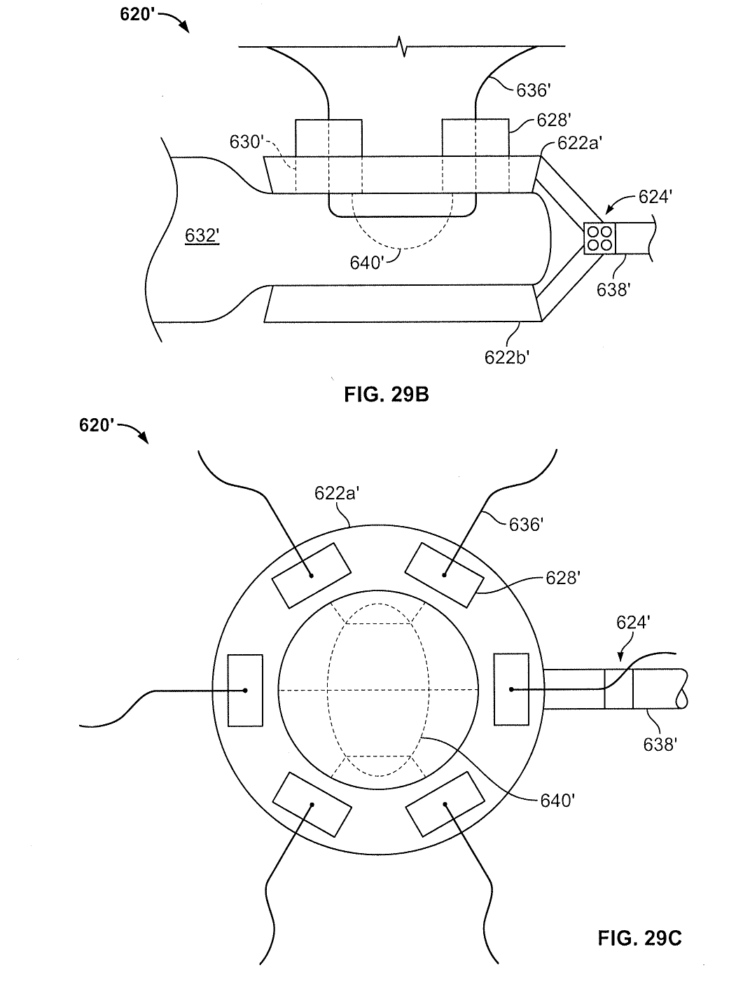

With reference to FIGS. 29A-C, an exemplary embodiment of a clamping forceps 620′ is provided for implementation with surgical suture clips 628′ for tying off sutures 636′ utilized in a surgical procedure for removal of a tumor 626′. The exemplary clamping forceps 620′ generally include a clamping mechanism 624′ and a shaft 638′. In particular, FIG. 29A illustrates an exemplary clamping forceps 620′ with the facility for surgical suture clips 628′ utilizing a thru-organ body suturing technique, while FIGS. 29B and C illustrate a same-side organ body suturing technique. With respect to FIG. 29A, the first clamp member 622a′, i.e., an anterior clamp, and the second clamp member 622b′, i.e., a posterior clamp, can generally be positioned to at least partially encircle the tumor 626′ or other structure. Surgical suture clips 628 can generally be inserted into voids 630′ located around the periphery of the first and second clamp members 622a′ and 622b′. In a thru-organ body suturing technique, sutures 636′ are generally passed through the surgical suture clips 628′ and the organ 632′ in a diametrically opposed manner as shown in FIG. 29A. In some exemplary embodiments, Surgicel®, for example, may be inserted into the ring area within the first clamp member 622a′ perimeter, i.e., on the tumor bed. The sutures 636′ and corresponding suture clips 628′ can then be tightened in the normal fashion, thereby generally minimizing blood loss during the procedure.

Turning now to FIGS. 29B and C, the first and second clamp members 622a′ and 622b′ are shown clamped around an organ 632′ and, in particular, with the inner perimeter of the first clamp member 622a′ surrounding the tumor bed 640′. In a same-side organ body suturing technique, surgical suture clips 628′ are generally inserted into voids 630′ of the first clamp member 622a′. Further, sutures 636′ can generally be passed through the appropriate surgical suture clips 628′ and into the organ 632′ such that the suture 636′ does not penetrate the organ 632′ from one side to the other. As would be understood by those of skill in the art, the suture 636′ can generally be passed through one surgical suture clip 628′, through a portion of tissue of the tumor bed 640′, and through another surgical suture clip 628′. In some exemplary embodiments, Surgicel®, for example, may be inserted into the ring area within the first clamp member 62a′ perimeter, i.e., on the tumor bed 640′. The sutures 636′ and corresponding suture clips 628′ can then be tightened in the normal fashion, thereby generally minimizing blood loss during the procedure.

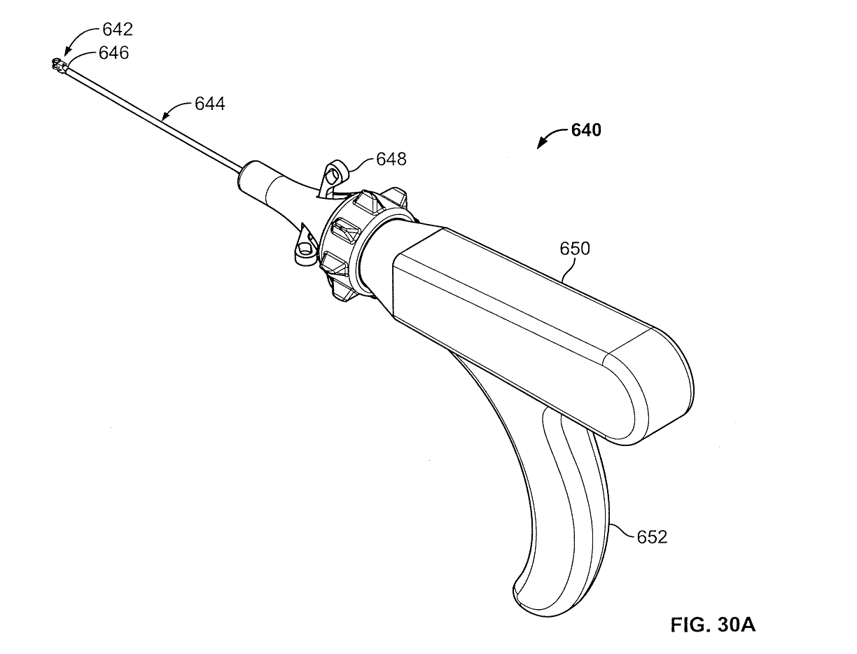

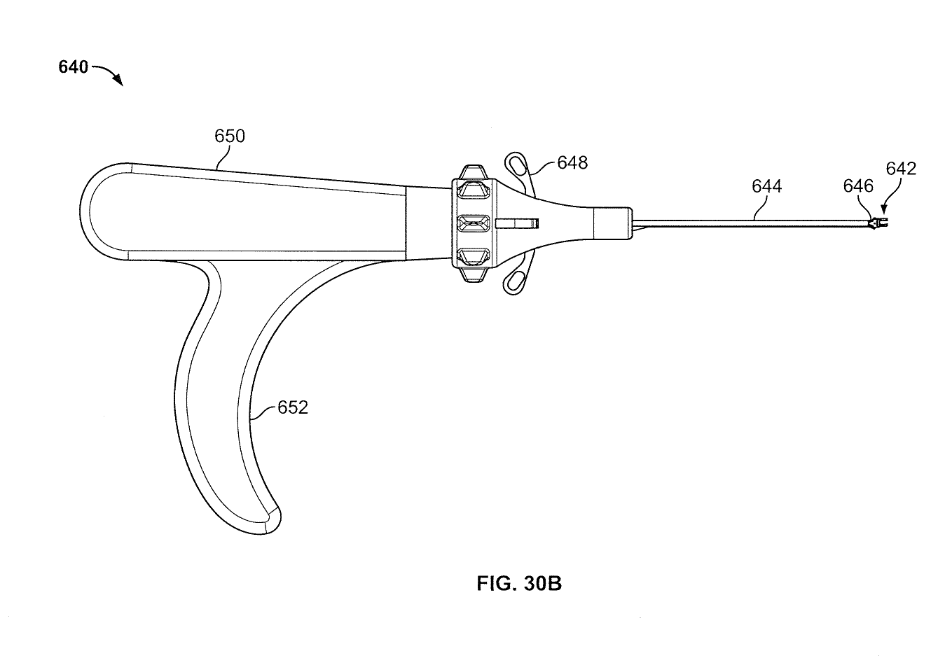

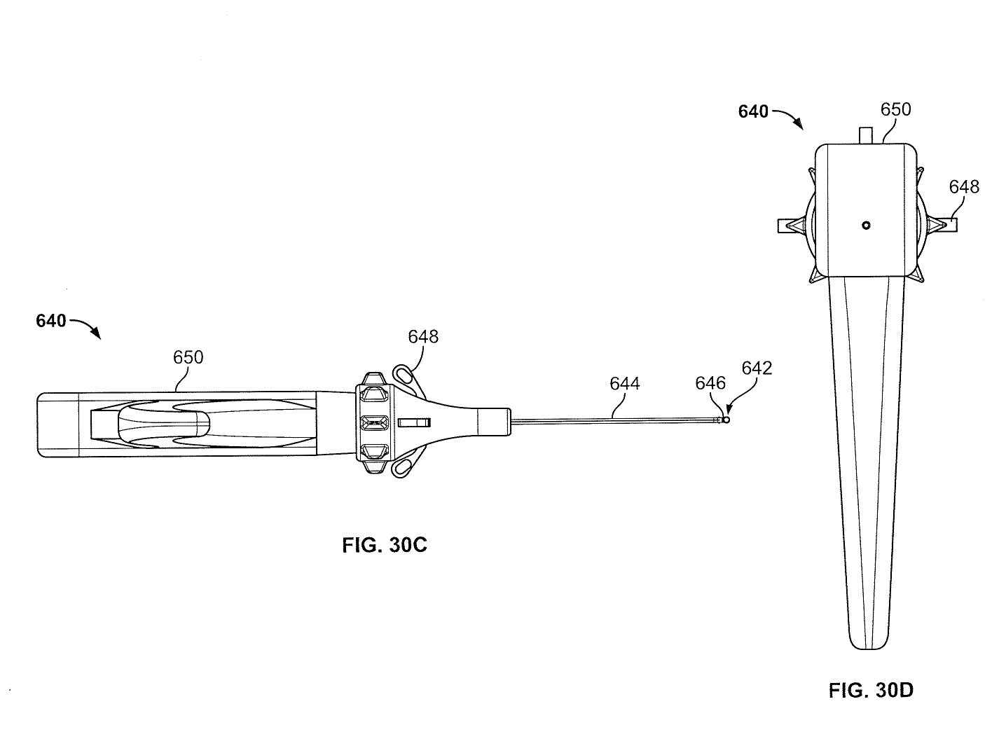

In accordance with embodiments of the present disclosure, an exemplary clamping forceps 640 is provided in FIGS. 30A-D for, e.g., manual, powered, motorized, and the like, laparoscopic introduction and/or actuation into a surgical site. The exemplary clamping forceps 640 is generally substantially similar in function to the previously discussed clamping forceps above. In particular, the clamping forceps 640 generally includes a head section 642, a clamping mechanism 646, an elongated body section 644 and a handle section 650. The head section 642 generally includes a first and second clamp member configured and dimensioned to cooperatively clamp so as to at least partially encircle a tumor or other structure. The clamping mechanism 646 is generally at least partially movably mounted with respect to the elongated body section 644. The handle section generally includes an articulation joint 648 and a grip 652.

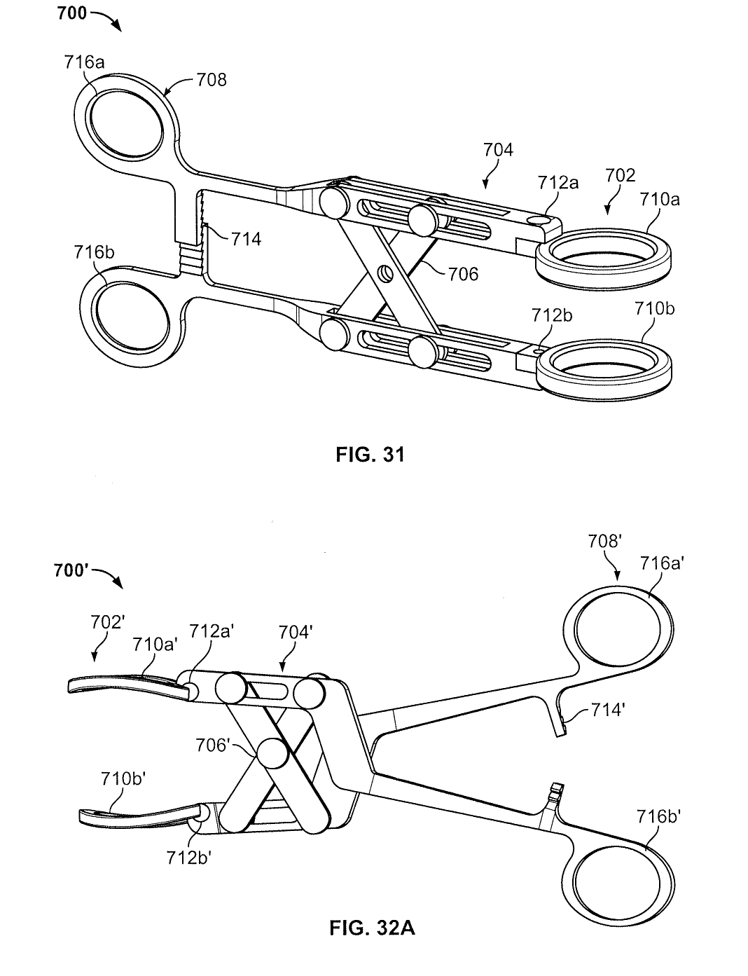

In accordance with further embodiments of the present disclosure, an exemplary clamping forceps 700 is provided in FIG. 31 for, e.g., manual open surgery operation. In particular, the clamping forceps 700 generally include a head section 702, an elongated body section 704, a clamping mechanism 706 and a handle section 708. The head section 702 generally includes first and second clamp members 710a and 710b, respectively, which may be detachably secured to the elongated body section 704 at first and second joints 712a and 712b. Thus, the first and second clamp members 710a and 710b may be disposable and/or interchanged with, e.g., alternative configurations, sizes, and the like, of clamp members. This customization of first and second clamp members 710a and 710b allows for appropriate adjustment based on, e.g., tumor size, and the like. In some exemplary embodiments, the first and second clamp members 710a and 710b may be substantially, e.g., 15 mm, 30 mm, 45 mm, 60 mm, and the like, in diameter. Although illustrated as having parallel clamping surfaces, it should be understood that in other exemplary embodiments, the clamping surfaces of the first and second clamp members 710a and 710b may be, e.g., curved, straight, angled, variable, and the like.

The clamping mechanism 706 can be, e.g., a spring-loaded mechanism, a gearing mechanism, a cable wire mechanism, a ratcheted mechanism, a motorized mechanism, a piezoelectric mechanism, a pneumatic mechanism, a solenoid actuator mechanism, a slide-crank mechanism, a slot yoke mechanism, a worm gear mechanism, a scissor mechanism, a rack and pinion mechanism, and the like. The clamping mechanism 706 can be configured such that actuation of the clamping mechanism 706 maintains substantially parallel clamping surfaces of the first and second clamp members 710a and 710b. The handle section 708 generally includes first and second finger holes 716a and 716b, e.g., intuitive thumb and ring finger grips, and a locking mechanism 714. In particular, the locking mechanism 714 may be, e.g., a ratchet locking mechanism 714, and generally allows a user to lock the first and second clamp members 710a and 710b in a desired position during a surgical procedure and further allows a user to unlock the first and second clamp members 710a and 710b in order to unclamp an organ after the surgical procedure has been completed.

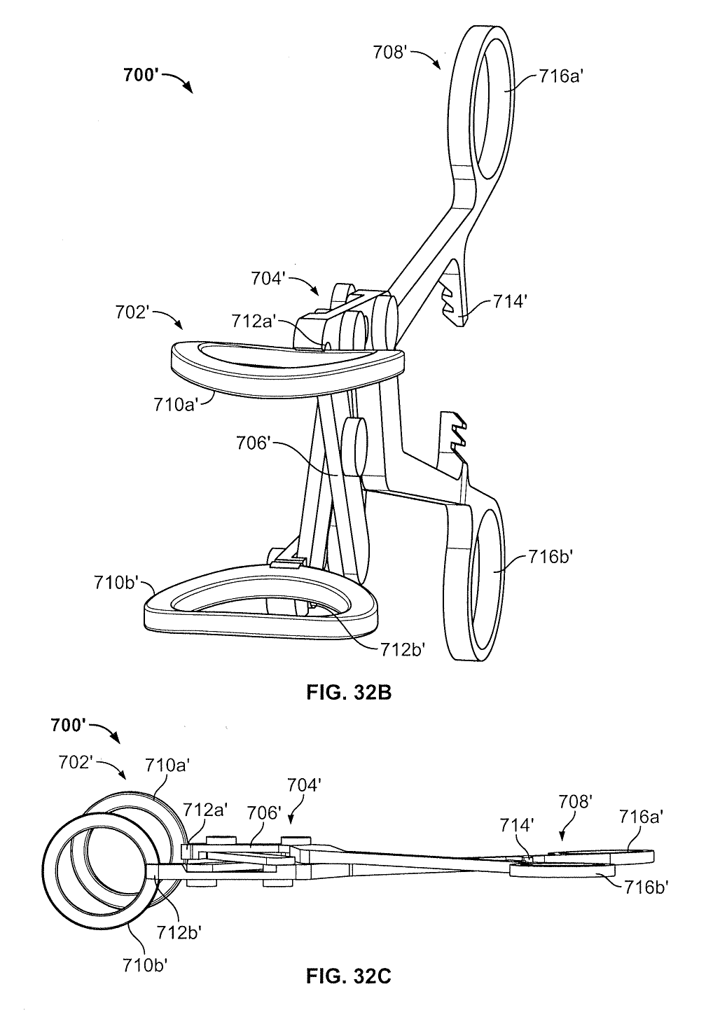

With reference now to FIGS. 32A-C, an exemplary embodiment of clamping forceps 700′ for manual open surgery operation is provided. The clamping forceps 700′ generally includes a head section 702′, an elongated body section 704′, a clamping mechanism 706′, and a handle section 708′. The first and second clamp members 710a′ and 710b′ are generally detachably secured to the elongated body section 704′ at first and second joints 712a′ and 712b′. The handle section 708′ generally includes first and second finger holes 716a′ and 716b′ and a locking mechanism 714′. In particular, the exemplary clamping forceps 700′ are substantially similar in structure and/or function to the clamping forceps 700, except for curved first and second clamp members 710a′ and 710b′. The curved first and second clamp members 710a′ and 710b′ may provide an even clamping surface and/or force distribution against a curved organ during clamping.

The clamping mechanism 706′ generally ensures substantially parallel actuation of the first and second clamp members 710a′ and 710b′ around an organ. As discussed above, the first and second clamp members 710a′ and 710b′ may be integrated with, e.g., a vacuum and/or fixation mechanism, sensors, high intensity focused ultrasound (HIM), therapeutic agents, and the like. In particular, it should be understood that the features and/or components discussed above with respect to the laparoscopic clamping forceps may be implemented in combination and/or separately with the manual open surgery clamping forceps discussed herein.

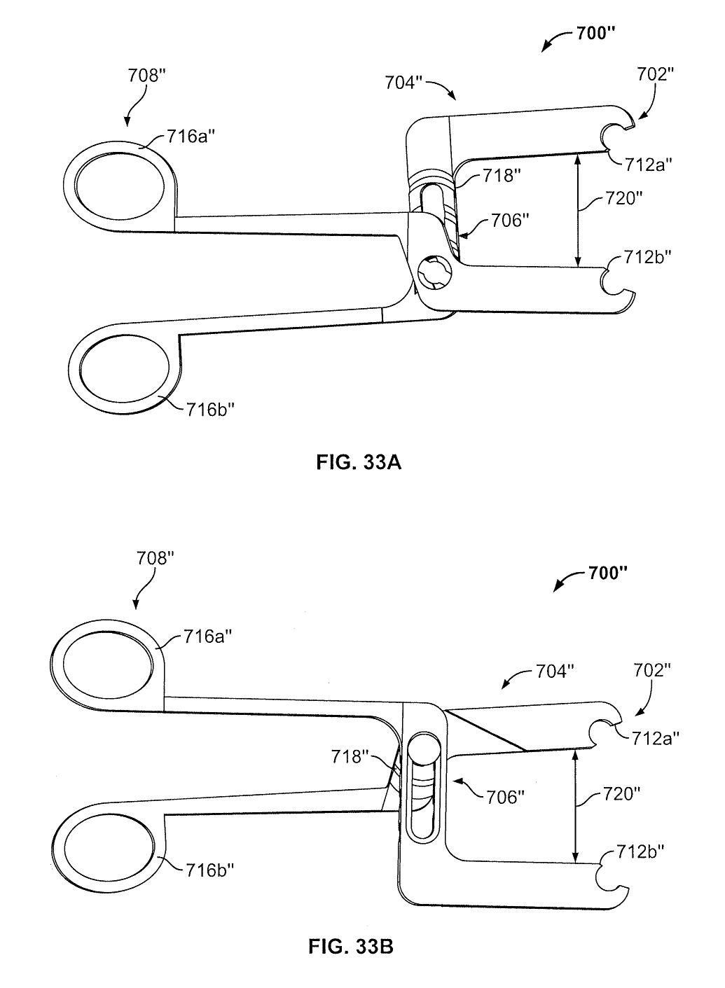

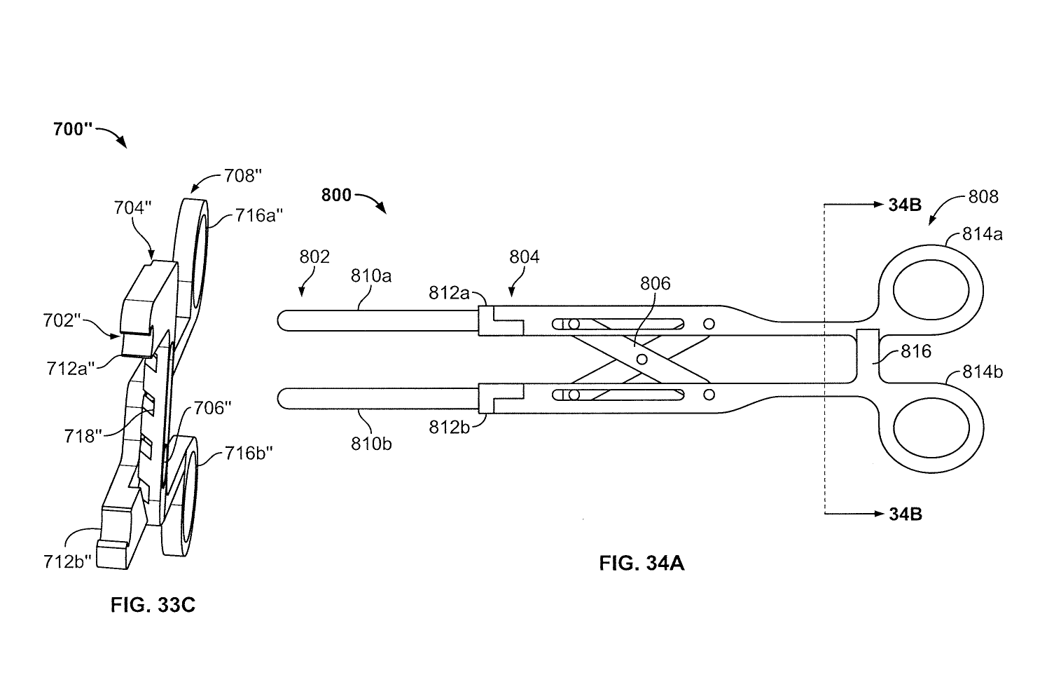

With reference to FIGS. 33A-C, an exemplary embodiment of clamping forceps 700″ for manual open surgery operation is provided. The clamping forceps 700″ generally includes a head section 702″, an elongated body section 704″, a clamping mechanism 706″, and a handle section 708″. Although illustrated without first and second clamp members, it should be understood that the head section 702″ generally includes detachably secured first and second clamp members. In particular, the first and second clamp members generally secure to and/or mate with the head section 702″ at first and second joints 712a″ and 712b″. The handle section 708″ generally includes first and second finger holes 716a″ and 716b″. The clamping mechanism 706″ generally includes a plurality of tracks 718″ which are configured and dimensioned to mate with a male member (not shown) in order to regulate the distance 720″ between the first and second clamp members. Thus, the distance 720″ between the first and second clamp members may be adjusted based on, e.g., the size of the organ to be clamped. Although illustrated with three tracks 718″, in other exemplary embodiments, the clamping mechanism 706″ may include, e.g., two, three, four, five, six, seven, eight, and the like, tracks 718″. FIGS. 33B and 33C illustrate additional side and front views of the exemplary clamping forceps 700″.

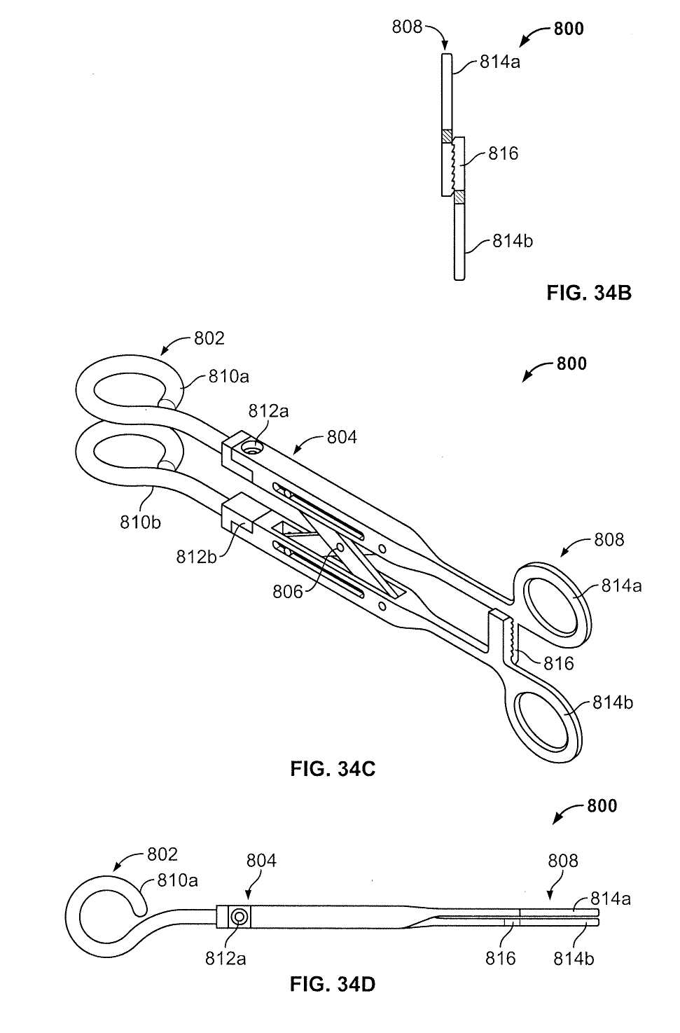

In accordance with exemplary embodiments of the present disclosure, a clamping forceps 800 is provided in FIGS. 34A-D for, e.g., manual open surgery, manual laparoscopic surgery, powered laparoscopic surgery, and the like. The clamping forceps 800 generally includes a head section 802, an elongated body section 804, a clamping mechanism 806, and a handle section 808. The handle section 808 generally includes first and second finger holes 814a and 814b and a locking mechanism 816. The locking mechanism 816 can include, e.g., interlocking ridges, and the like, for locking the first and second clamp members 810a and 810b relative to each other in a clamped orientation around an organ.

The first and second clamp members 810a and 810b are generally detachably secured to the elongated body section 804 at first and second joints 812a and 812b. Further, the first and second clamp members 810a and 810b can generally be malleable in order to permit the user to form the first and second clamp members 810a and 810b into a variety of configurations. The first and second clamp members 810a and 810b may be fabricated from, e.g., a moldable polymer, and the like. In some exemplary embodiments, the clamping forceps can initially be substantially straight and transmitted via a trocar port to the insufflated body. Heat, for example, may be utilized to soften the material of the first and second clamp members 810a and 810b such that the first and second clamp members 810a and 810b may be manipulated and/or formed into a desired configuration, e.g., the contour of the tumor, and the like. The manipulation of the first and second clamp members 810a and 810b may be performed via, e.g., a user interface, physical force applied to mold the first and second clamp members 810a and 810b via laparoscopic forceps, and the like. In some exemplary embodiments, cold forming via, e.g., a set of laparoscopic forceps, a movable arm, and the like, may be utilized to form the first and second clamp members 810a and 810b into the desired configuration. Upon completion of the surgical procedure, the first and second clamp members 810a and 810b can be returned to a substantially straight configuration such that they may be removed from the insufflated cavity through a trocar.

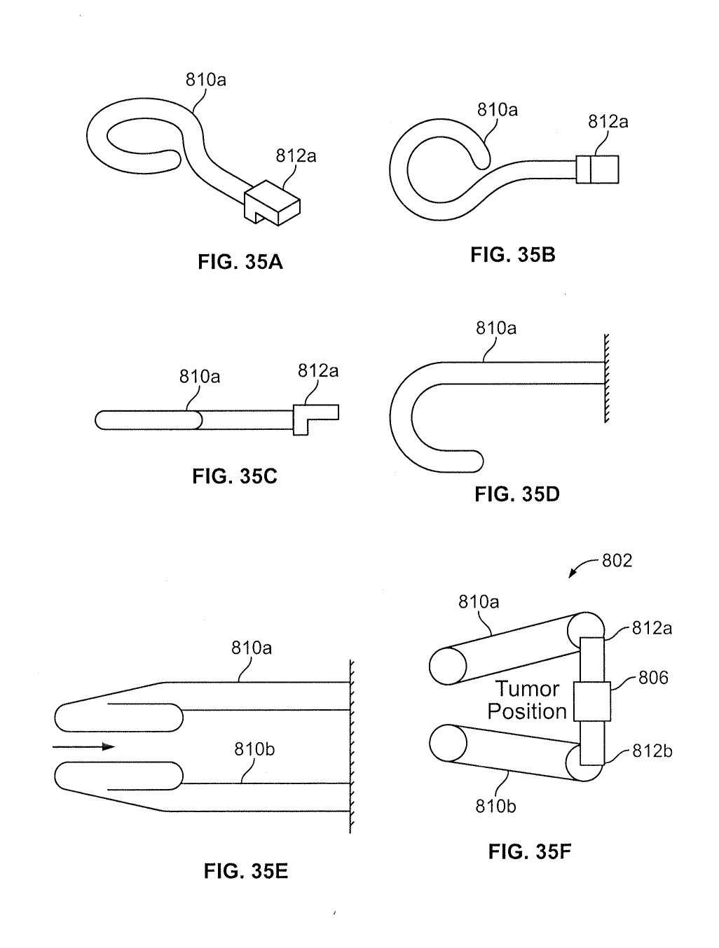

FIGS. 34B-D illustrate a sectional view at point B, perspective and top views, respectively, of the exemplary clamping forceps 800. In particular, FIGS. 34C and D illustrate the clamping forceps 800 with first and second clamp members 810a and 810b configured as substantially round clamping members. With reference to FIGS. 35A-F, exemplary first and second clamp members 810a and 810b of the exemplary clamping forceps 800 are illustrated in a plurality of configurations. It should be understood that the configurations of the malleable first and second clamp members 810a and 810b are provided herein for illustration purposes only and that in other exemplary embodiments, the first and second clamp members 810a and 810b can be molded into, e.g., rectangular, oval, square, polygonal, and the like, configurations.

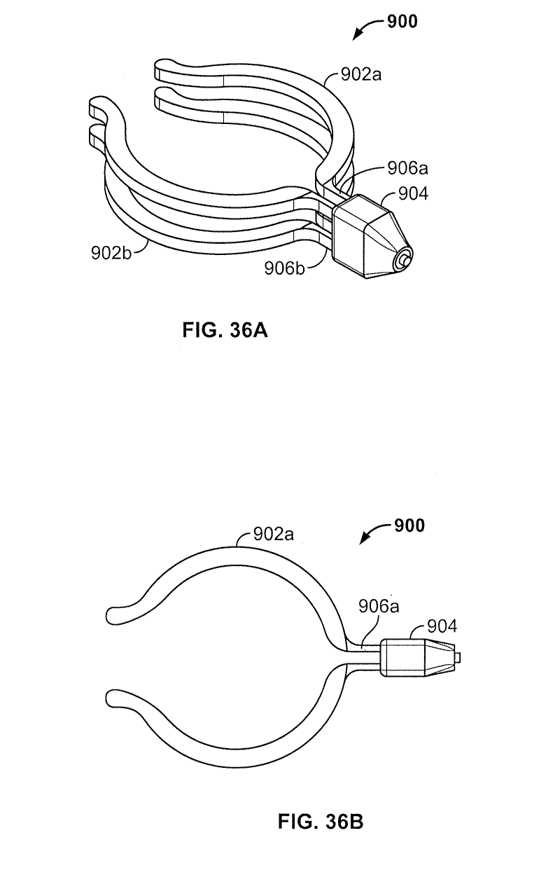

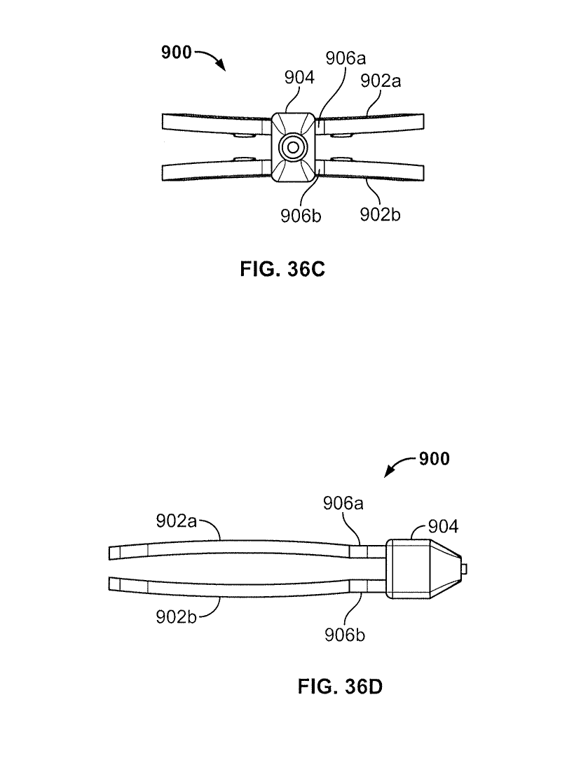

In accordance with embodiments of the present disclosure, an exemplary self-opening head section 900 for implementation in conjunction with the clamping forceps discussed herein is provided in FIGS. 36A-D. In particular, the exemplary section 900 generally includes first and second clamp members 902a and 902b and a clamping mechanism 904, e.g., a collar, and the like. The first and second clamp members 902a and 902b may be fabricated from a flexible material which enables transmission of the head section 900 via a trocar port/cannula. The first and second clamp members 902a and 902b may further be spring-loaded to open into a substantially expanded configuration once the clamping mechanism 904 has been translated such that the first and second clamp members 902a and 902b are exposed. The first and second clamp members 902a and 902b can generally include, e.g., flexible materials of fabrication, be hinged in the x-y plane and stiff in the z-plane, and the like. The clamping mechanism 904 can generally be implemented to actuate the first and second actuation elements 906a and 906b of the first and second clamp members 902a and 902b in order to clamp around an organ or other structure. As would be understood by those of ordinary skill in the art, as the clamping mechanism 904, e.g., a collar, is translated distally along the first and second actuation elements 906a and 906b, the clamping pressure between the first and second clamp members 902a and 902b generally increases and/or decreases progressively.

Although illustrated as open-ended first and second clamp members 902a and 902b, in other exemplary embodiments, the first and second clamp members 902a and 902b may be substantially closed to completely clamp around a tumor or other structure. Further, in other exemplary embodiments, the first and second clamp members 902a and 902b can be, e.g., square, rectangular, oval, polygonal, angled, variable, rounded to mate with the topography of an organ, and the like. FIGS. 36B-D illustrate top, back and side views of the exemplary head section 900 as discussed above.

With respect to the exemplary clamping forceps discussed herein, an exemplary process or method for use may include some or all of the steps below. The user generally brings the clamping forceps into close proximity to the targeted surgical location. As the first and second clamp members are applied to the tissue body, the clamping and/or closing pressure of the first and second clamp members is generally precisely controlled. In some exemplary embodiments, the user may be able to present a Doppler ultrasound probe to the surgical area in order to determine whether or not blood of the tissue body or organ is flowing. For example, this may be determined by the frequency of audible beeps emitted by a standard ultrasound unit. In conjunction with the Doppler ultrasound probe, the user generally actuates the first and second clamp members to precisely close the first and second clamp members around the organ and/or tumor in the desired location. In the exemplary embodiments where a user desires restricted blood flow, as the Doppler ultrasound probe slows and subsequently ceases to emit an audible beeping noise, the user generally stops further actuation and clamping of the first and second clamp members. At this point, the clamping forceps are considered to have applied sufficient pressure to restrict and/or stop the blood flow to the tumor without crushing the organ and/or tissue body. However, blood flow to the remainder of the organ outside of the clamped area generally continues to flow unimpeded. In some exemplary embodiments, a suction mechanism may be utilized to draw in tissue around the surgical site in order to accurately fixate the first and second clamp members to the precise location.

The user, e.g., the surgeon, further generally excises the tissue and/or tumor through an access orifice, i.e., the inner perimeter of the first and second clamp members. The excised tissue may be removed from the patient through, e.g., retraction, suction, and the like, via the orifice in the clamping forceps. Upon excising a satisfactory negative surgical margin, the user generally closes the void resulting from the excision in the manner generally implemented in the industry. For example, the user may implement surgical suture clips, as discussed above, to tie off the suture. The tumor bed is therefore closed via, e.g., needle and suture, and the sides of the void are generally approximated as closely as possible. Surgical suture clips may be applied to the suture to reduce the risk of tearing of the parenchymal tissue and appropriate knots are generally tied to secure the suture. In some exemplary embodiments, the Doppler ultrasound probe may be further implemented to ensure confidence in the closure and/or blood flow to the organ. The suction mechanism may also be released to draw out the tissue surrounding the surgical site. Once a user is satisfied with the closure of the void, the clamping forceps may be released from the clamped position around the organ by, e.g., a quick release mechanism, a precisely controlled gearing mechanism, and the like. The exemplary clamping forceps are generally removed from the body of the patient and additional Doppler ultrasound probes may be implemented to again confirm proper blood flow and/or normal operation of the organ.

In some exemplary embodiments, the surgical suture clips and the suture can be positioned in the manner described herein and Surgical® bolster and/or another hemostatic agent may be inserted into the tumor bed. The exemplary clamping forceps can generally be removed and the surgical suture clips can generally be tightened via, e.g., the Sliding Clip Renorrhaphy surgical technique of pulling up on the suture with one hand and pushing down on the surgical suture clip with the other so as to exert maximum pressure on the organ, i.e., kidney, and establish hemostasis. (See, e.g., Bhayani, S. B. et al., The Washington University Renorrhaphy for Robotic Partial Nephrectomy: a detailed description of the technique displayed at the 2008 World Robotic Urologic Symposium, Journal of Robotic Surgery, 2(3), p. 139-140 (2008); and Benway, B. M. et al., Robotic Partial Nephrectomy with Sliding-Clip Renorrhaphy: technique and outcomes, European Urology, 55(3), p. 592-599 (2009)). Suture knots can then be implemented to fixate the surgical suture clips in the desired location and hold the tumor bed closed.

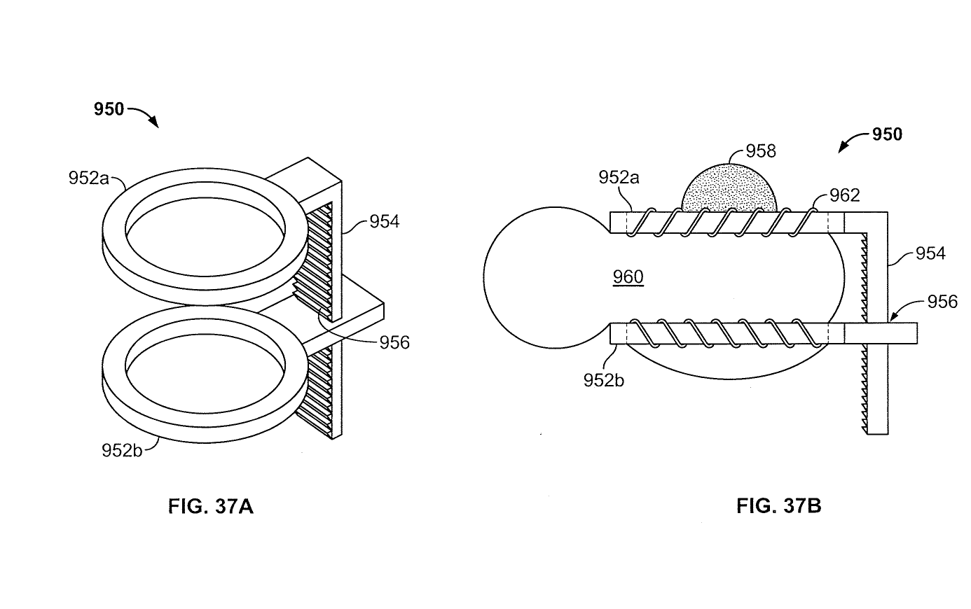

Turning now to FIGS. 37A and B, an exemplary bioresorbable clamp 950 for implementation with the exemplary clamping forceps described herein is provided. In some exemplary embodiments, the exemplary clamp 950 can be applied via, e.g., laparoscopic forceps, and the like. The clamp 950 may be configured as, e.g., circular, oval, rectangular, square, C-shaped, J-shaped, and the like. The exemplary clamp 950 can generally be fabricated from bioresorbable biomaterials certified by the FDA for medical devices such as, e.g., polydioxanone, poly-lactic-glycolic acid, glycolide, lactide, caprolactone, trimethylene carbonate, polyethylene glycol, and the like. In some exemplary embodiments, the clamp 950 can be manufactured from a biomaterial including, e.g., one of the exemplary materials listed above, a combination of the exemplary materials listed above, alternative biomaterials, and the like. In particular, the materials of fabrication of the clamp 950 can generally be resorbed intra-corporeally over time via normal body processes, e.g., hydrolysis, enzymatic degradation, and the like, and subsequently excreted from the body via normal body processes, e.g., urinary excretion, and the like.

The exemplary clamp 950 generally includes a first clamp member 952a and a second clamp member 952b configured and dimensioned to be positioned to at least partially encircle a tumor or other structure. Although illustrated as solid first and second clamp members 952a and 952b, in some exemplary embodiments, the first and second clamp members 952a and 952b may be fabricated from a plurality of interconnected linkages and thereby be introduced into a surgical site in a substantially folded manner through a trocar. The first clamp member 952a can generally be movable with respect to the second clamp member 952b. In particular, the first clamp member 952a generally includes a clamping mechanism 954, e.g., a ratchet mechanism, a snap clip, and the like, that mates and/or otherwise fits into a complementary aperture 956 located on the second clamp member 952b. In some exemplary embodiments, the clamp 950 may be implemented in laparoscopic and/or open surgery and can be actuated and/or introduced into the surgical site by implementing the exemplary clamping forceps discussed herein.

In particular, during a surgical procedure, e.g., a partial nephrectomy, a partial hepatectomy, and the like, the clamp 950 can generally be positioned around an organ 960, e.g. a kidney, liver, and the like, such that the first and second clamp members 952a and 952b at least partially encircle a tumor 958 or other structure/tissue. Upon proper positioning, the user, e.g. a surgeon, can generally proceed to compress the first and second clamp members 952a and 952b in a clamping manner, thereby exerting pressure on the organ 960. The user can generally further continue to compress the clamp 950 until blood flow to the tumor 958 has been substantially and/or completely restricted. The restriction of blood flow to the tumor 958 can generally be confirmed through the utilization of generally available Doppler ultrasound sensing instruments and/or the sensors discussed herein. Upon confirmation of the stoppage of blood flow to the tumor 958, an absorbable suture 962 can be wrapped around the clamp 950 so as to fixate its position relative to the organ 960. For example, the suture 962 may be wrapped around the first and second clamp members 952a and 952b. In some exemplary embodiments, the absorbable suture 962 may be applied utilizing the generally available interrupted, running, mattress, and/or other suturing techniques in the industry and secured via, e.g., standard suture knots, and the like. As would be understood by those of ordinary skill in the art, the clamp 950 and the suture 962 can generally be left in the patient's body where, over time, the tumor 958 generally dies via necrosis resulting from the blocked and/or restricted blood flow and can be subsequently resorbed via normal body processes. Over a greater period of time, i.e., after the tumor 958 has resorbed, the exemplary clamp 950 and absorbable suture 962 can generally also be resorbed via normal body processes, e.g., hydrolysis, and the like, and subsequently excreted from the body via normal urinary processes. In some exemplary embodiments, the tumor 958 can be, e.g., excised, ablated, otherwise removed, and the like, via the processes discussed herein.

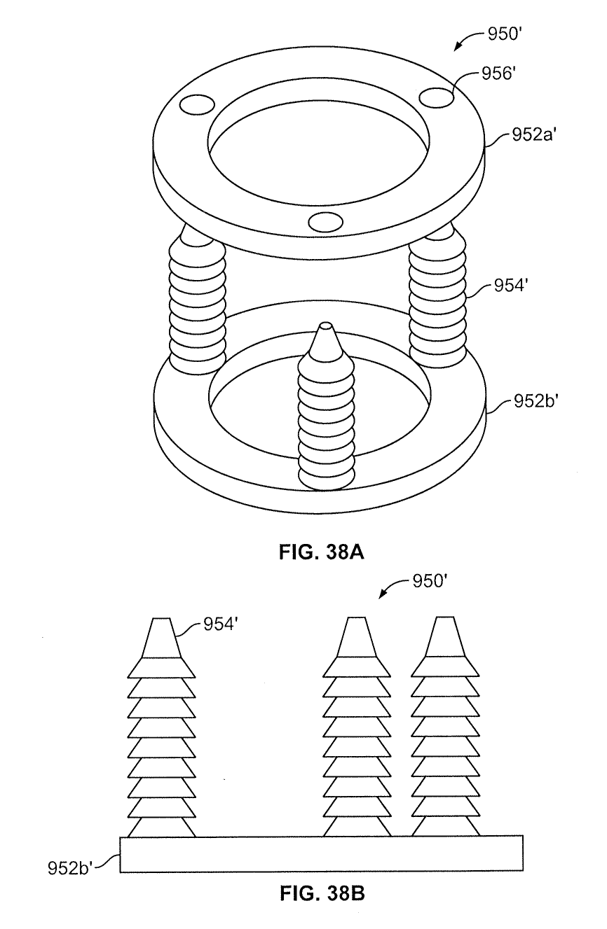

With reference to FIGS. 38A and B, another exemplary embodiment of a bioresorbable clamp 950′ is provided. In particular, the exemplary clamp 950′ generally includes a first clamp member 952a′ and a second clamp member 952b′. The exemplary clamp 950′ generally includes a clamping mechanism, i.e., at least one extension 954′ and at least one aperture 956′ configured and dimensioned to mate with each other. Although illustrated with three extensions 954′ and three apertures 956′, in some exemplary embodiments, e.g., one, two, three, four, five, six, seven, eight, and the like, extensions 954′ and apertures 956′ can be used. The extension 954′ generally extends from the second clamp member 952b′ in a substantially perpendicular manner and defines a plurality of, e.g., ribs, teeth, and the like. As would be understood by those of ordinary skill in the art, the ribbed extensions 954′ can generally be inserted and/or pressed into the apertures 956′ such that the first and second clamp members 952a′ and 952b′ are substantially interlocked relative to each other and/or an organ. Similar to the clamp 950 described above, the first and second clamp members 952a′ and 952b′ can generally be positioned around an organ to at least partially encircle a tumor or other structure. The clamp 950′ can generally be further interlocked by a surgeon such that blood flow to the encircled tumor is partially and/or fully restricted. Over time, the tumor generally dies via necrosis and the tumor and clamp 950′ may be resorbed via normal body processes.

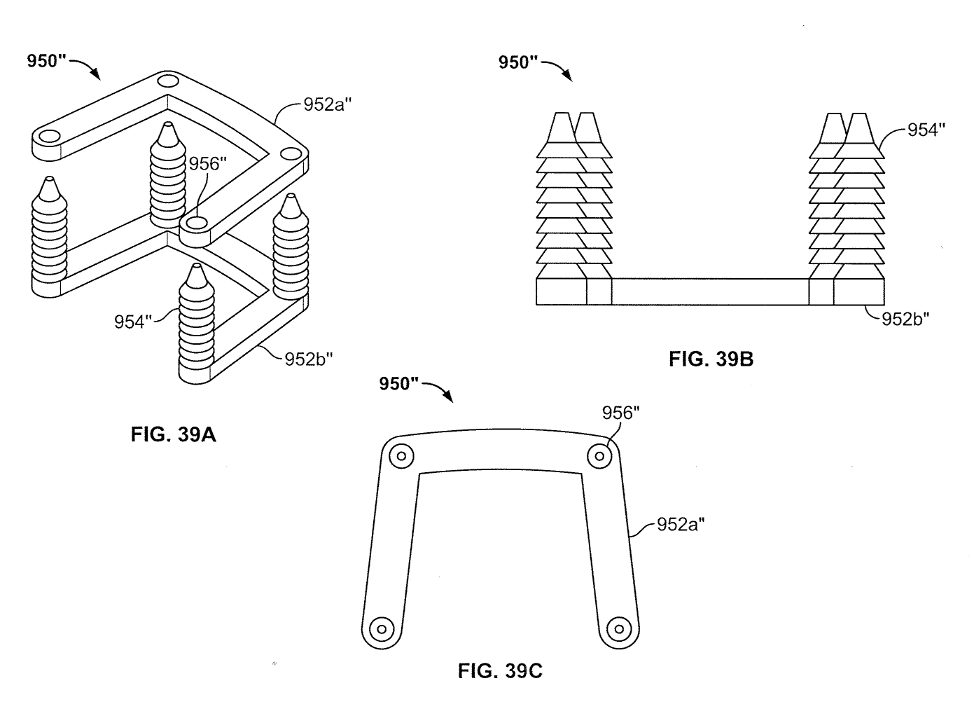

With reference to FIGS. 39A-C, an exemplary embodiment of a bioresorbable clamp 950″ is provided and is generally substantially similar in function as the clamp 950′ described above. In particular, the exemplary clamp 950″ generally includes a first clamp member 952′a″ and a second clamp member 952b″. In general, the exemplary clamp 950″ generally includes extensions 954″ and complimentary apertures 956″ configured and dimensioned to interlock. As described above, the first and second clamp members 952a″ and 952b″ can generally be interlocked around, e.g., a tumor, and the like, by compressing the first and second clamp members 952a″ and 952b″ such that the ribbed extensions 954″ and the apertures 956″ interlock. The desired clamping pressure is thereby maintained by the interlocked clamp 950″ for a desired period of time, i.e., until the tumor has died via necrosis. The bioresorbable clamp 950″ and the tumor can generally resorb via normal body processes. Although illustrated as substantially C-shaped, in some exemplary embodiments, the clamp 950″ can be configured as substantially, e.g., circular, oval, square, rectangular, C-shaped, J-shaped, variable, and the like.

In accordance with further aspects of the present disclosure, clamping forceps are provided that include first and second clamping members that are angularly oriented with respect to the operative handle section to facilitate surgeon viewing and positioning of the clamping members relative to a desired anatomical location/region. The clamping members may define advantageous geometric configurations to facilitate positioning relative to an anatomical location/region. For example, angular joints may be provided such that the clamping members define advantageous geometric configurations, e.g., a substantially trapezoidal configuration, a compound curvature configuration, and the like. The angular joints or transitions may be fixed, e.g., during clamping forceps fabrication, or variable at the time of surgery. The elongated body section that extends between the handle section and the clamping members may include a clamping mechanism and may define, in whole or in part, a substantially curved configuration to further enhance surgeon visibility and positioning of the clamping members, e.g., when the surgical procedure is performed by way of a flank incision. Thus, a curved region in the transition from the handle section to the elongated section may be advantageously incorporated into the clamping forceps design. The curved region may be fixed, i.e., established during fabrication of the clamping forceps, or variable such that the surgeon may select a desired curve for a specific procedure and then “fix” the selected curve for completion of the surgical procedure.

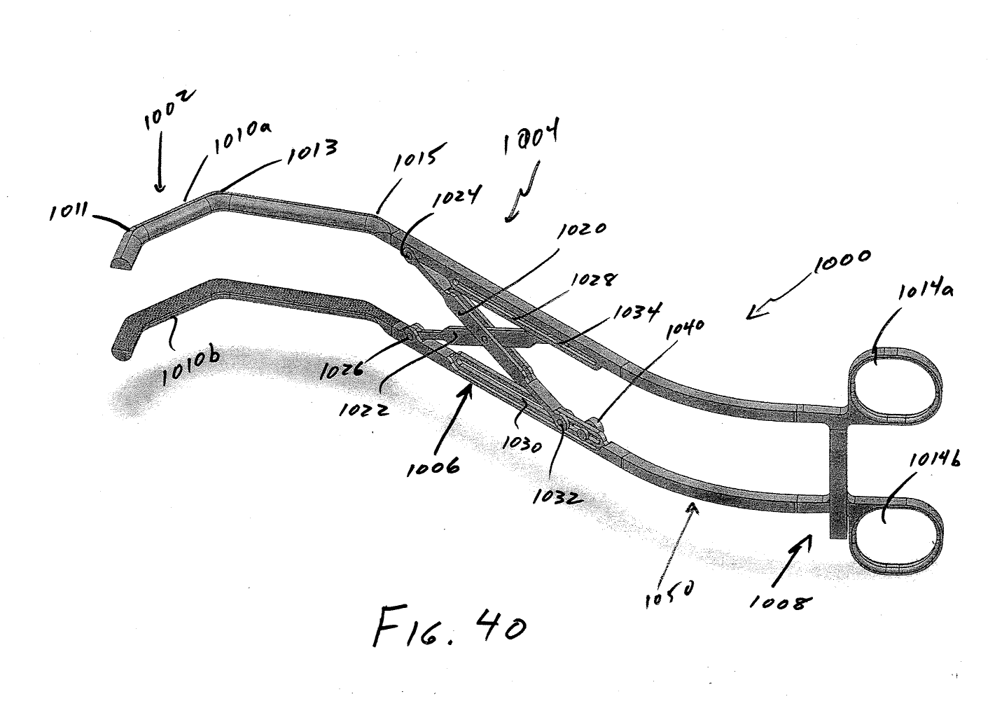

Thus, with reference to FIGS. 40-43, a further exemplary clamping forceps 1000 is provided. Clamping forceps 1000 is generally similar to clamping forceps 700 and 800 described with reference to FIGS. 31, 32A-C, and 34A-D, but includes advantageous features and functions relative to the previously disclosed embodiment. Clamping forceps 1000 is particularly adapted for manual open surgery and includes a head section 1002, an elongated body section 1004, a clamping mechanism 1006, and a handle section 1008. The handle section 1008 generally includes first and second finger holes 1014a and 1014b, and a locking mechanism 1016 (best seen in FIG. 42). The locking mechanism 1016 can include, e.g., interlocking ridges/ratchet teeth, and the like, for locking the first and second clamp members 1010a and 1010b relative to each other in a clamped orientation around an organ. The ability to provide progressive clamping pressure may be effectuated by providing a progressively finer pitch to the ratchet teeth. Because of the sensitivity of the kidney, it is important to have fine control of the degree of approximation of the clamping members 1010a, 1010b at times of higher clamping pressure. This is important at least in part because the surgeon needs to control his approximation activities so as to avoid tearing the parenchyma, while still limiting the blood flow rate, and finer ratchet teeth support a desired level of control. The first and second clamp members 1010a and 1010b may be detachably secured to the elongated body section 1004, e.g., as described with reference to clamping forceps 700 and 800.

Clamping mechanism 1006 includes cooperating scissor arms 1020, 1022 that are fixed at one end relative to the elongated body section 1004 at anchor points 1024, 1026, respectively. As shown in FIG. 40, scissor arm 1020 defines a yoke-like structure within which scissor arm 1022 travels, thereby providing greater structural stability to clamping mechanism 1006. Guide slots 1028, 1030 are defined by elongated body section 1004 and guide pins 1032, 1034 are associated with the “free” ends of scissor arms 1020, 1022, respectively. As guide pins 1032, 1034 travel proximally relative to guide slots 1028, 1030 and elongated body section 1004, the clamping members 1010a, 1010b are brought into substantially parallel approximation. Positioning of the anchor points 1024, 1026 in distal locations relative to guide slots 1028, 1030 ensures that the scissor arms 1020, 1022 are not impeded by tissue/organs as the clamping members 1010a, 1010b are brought into approximation because the travel of guide pins 1032, 1034 is in a proximal direction during the approximation process, i.e., away from the tissue/organ to be clamped.

Of note, one or more repositionable backstops 1040, e.g., thumbscrew backstops, may be provided for manual positioning with respect to at least one of the guide slots 1028, 1030. In the absence of a repositionable backstop 1040, the degree to which clamping members 1010a, 1010b may be brought into approximation is guided or controlled by three factors: (i) the degree to which a surgeon compresses the handle section 1008, (ii) the thickness and overall resistance to compression exerted by the structure positioned between the clamping members 1010a, 1010b, and (iii) the available travel distance defined by guide slots 1028, 1030. By manually positioning repositionable backstop(s) 1040 relative to guide slot(s) 1028 and/or 1030, the surgeon is able to limit the degree to which the clamping members 1010a, 1010b are approximated on a selective basis.

In practice, the ability of a surgeon to control the degree to which clamping members 1010a, 1010b may be approximated, e.g., based on the size/thickness of the kidney, may be of clinical benefit. For example, a typical off-clamp partial nephrectomy results in 600-800 cc in estimated blood loss over a 20-30 minute procedure, or an average rate of approximately 20-40 cc/min through the clamped incision. By clamping on the parenchyma, the disclosed clamping forceps significantly reduces blood loss while still providing the benefits of an off-clamp procedure, i.e., no warm ischemia time. However, the kidney is a sensitive organ and care must be exercised during clamping so as not to institute tissue trauma on the clamped organ. By providing manually repositionable backstop(s) 1040, positioning of backstop(s) 1040 relative to the slot(s) 1028 and/or 1030 limits the distance between the approximated clamp members 1010a, 1010b and, in effect, limits the clamping force exerted on the kidney. Furthermore, the position of the backstop(s) 1040 limits the blood flow rate passing thru the cross-section of the parenchyma that is exposed upon excision of the tumor, e.g., to less than 10 cc/min. Some blood flow is necessary in order to see trickles of blood flow and to thereby locate the place(s) where additional suturing needs to be completed. Locating these positions intra-operatively and effectuating additional suturing is important to reduce the risk of post-op internal hemorrhages. Thus, the disclosed clamping forceps that facilitates controlled clamping of the desired anatomical location/region is advantageous because, inter diet, it enables holding of the tissue, but not full clamping of the organ, thereby controlling the blood flow rate throughout the clamped incision.

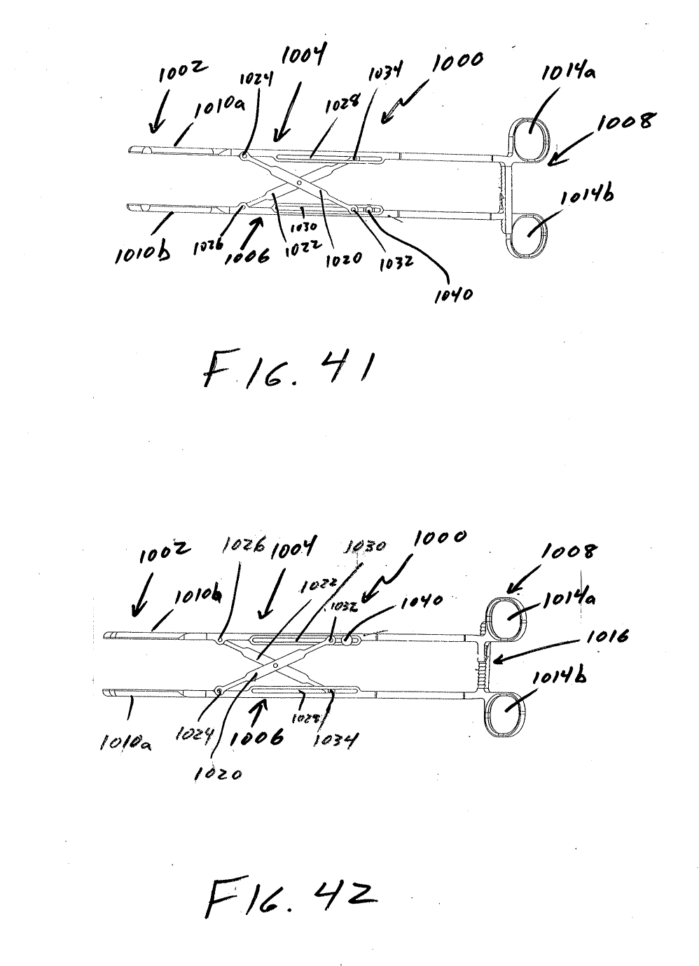

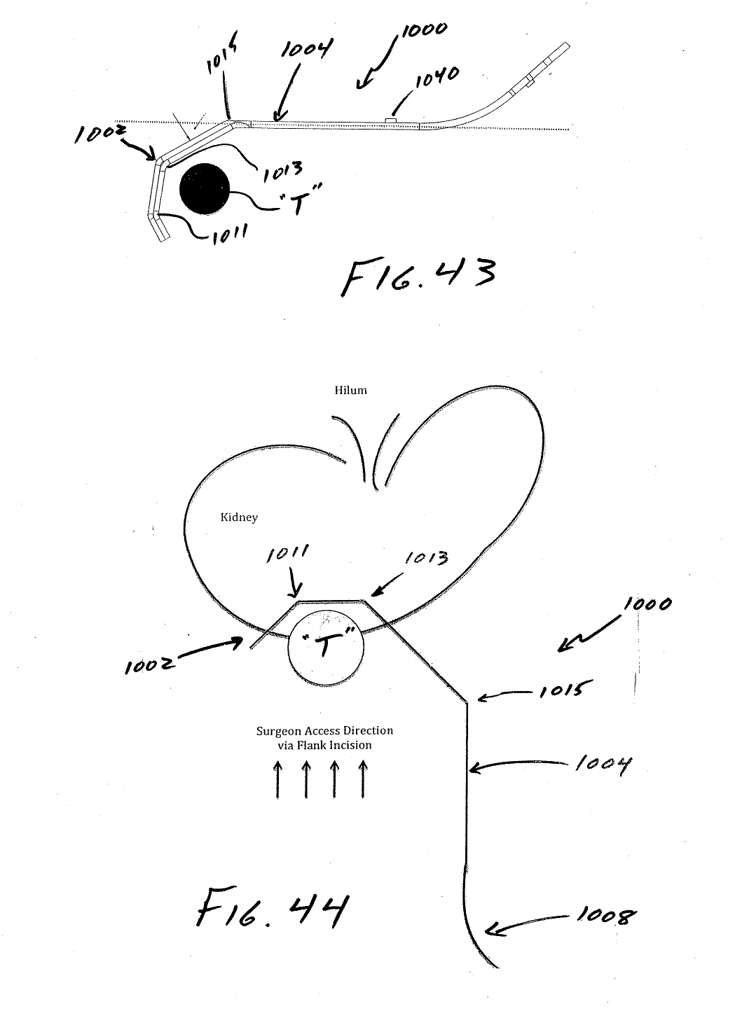

First and second clamp members 1010a and 1010b are angled relative to elongated body section 1004 so as to facilitate surgeon visibility and clamping relative to a desired anatomical region/location, e.g., so as to beneficially capture a tumor and required margin within the clamping confines. Thus, in exemplary embodiments of the present disclosure, each of first/second clamp members 1010a, 1010b define first and second joints 1011, 1013, such that the head section 1002 defines a substantially trapezoidal configuration when viewed from the side (see, e.g., FIGS. 43 and 44). The first and second joints 1011, 1013 may define substantially equal angular transitions, e.g., between 15° and 60°, and preferably about 45° each. Thus, if both the first and second joints 1011, 1013 define approximately 45° angular transitions. The foregoing angular transitions at joints 1011, 1013 may be fixed, i.e., established during fabrication of the disclosed clamping forceps, or may be adjustable during use. Thus, a level of flexibility may be imparted to the first and/or second joints 1011, 1013 based on material selection to facilitate refinement of the angular transitions based on specific clinical factors, e.g., the size and/or geometry of the kidney and/or tumor.

As shown in FIGS. 40 and 44, a further joint 1015 may be advantageously defined at or in the vicinity of the transition from the head section 1002 to the body section 1004 to further facilitate surgeon visibility, ergonomics and positioning of the head section 1002 relative to a desired anatomical region/location. The angular transition associated with further joint 1015 generally ranges between 0° and 60°, from the axis of the elongated body section 1004, and is preferably about 45′. As with joints 1011, 1013, further joint 1015 may be fixed during fabrication of the disclosed clamping forceps or adjustable at the time of use, e.g., based on material selection or articulation/rotational mechanism(s) in the region of further joint 1015. Joints 1011, 1013, 1015 cooperate to allow the first and second clamp members 1010a, 1010b to be effectively positioned relative to a desired anatomical location/region with advantageous surgeon visibility.

It is to be noted that the present disclosure provides advantageous clamping member geometry for partially encompassing, but fully isolating, target anatomical structures, e.g., tumors and the like. Thus, for example, clamping forceps 1000 provide a substantially trapezoidal clamping member geometry that is effective to surround and isolate a tumor, while not fully surrounding or encircling the tumor. The ability to isolate a tumor, as described herein, is highly advantageous from a clinical standpoint, as will be readily apparent to persons skilled in the art.

Beyond the angled joints previously described with reference to clamping forceps 1000, a further angled effect is advantageously associated with clamping forceps 1000. As shown in FIG. 40, elongated body section 1004 includes an arcuate region 1050 that further facilitates visibility of the clamping members in use. The precise arc defined in arcuate region 1050 may vary, but is generally selected so as to enhance visibility of the clamping members during clinical procedures, as will be apparent to persons skilled in the art. Of note, arcuate region 1050 does not interfere with the design and/or operation of clamping mechanism 1006, which is generally associated with a non-arcuate region of elongated body section 1004.

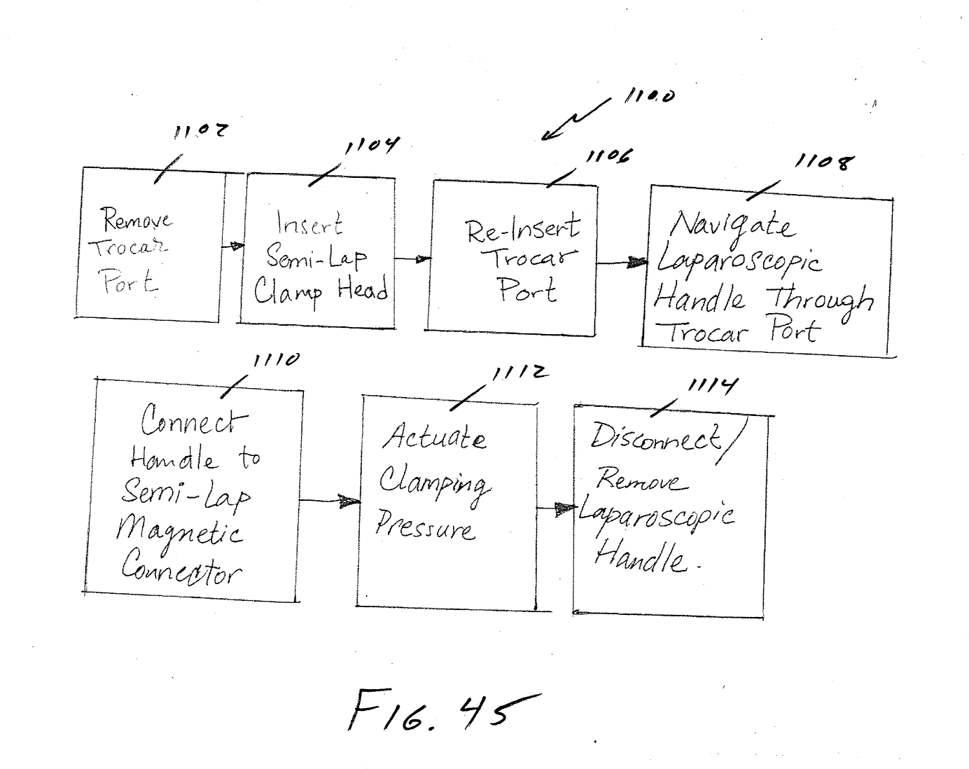

The advantageous ability to position clamping members 1010a, 1010b relative to a desired anatomical location/region is schematically illustrated in FIGS. 43 and 44. As shown therein, the substantially trapezoidal geometry of head section 1002 is effective in partially encompassing and isolation a tumor “T” with an effective margin internal thereof. With reference to FIGS. 45-48, exemplary implementations of the present disclosure are provided wherein clamping forceps 1200 offers hybrid functionality, i.e., functionality that benefits from aspects of an “open” surgical procedure and aspects of a “laparoscopic” or “minimally invasive” procedure. Thus, as shown in the flowchart of FIG. 45, an exemplary clinical implementation 1100 of a disclosed clamping forceps according to the present disclosure may include a surgeon introducing the clamping members to a surgical region (step 1104), e.g., through an incision, generally less than 0.5 inches in size, that was originally used in connection with the trocar port/cannula site. Step 1104 may optionally be performed after removal of a trocar port (step 1102). Thereafter, the surgeon may connect the clamping members to an elongated body section/handle section subassembly that is introduced through a trocar port/cannula (step 1110). The connection may take place after reinsertion of the trocar port (step 1106) and navigation of the elongated body section/handle section subassembly through the trocar port (step 1108). Intra-corporeal connection of the clamping members relative to the elongated body section/handle section subassembly is accomplished through a mating mechanism, e.g., a magnetic mechanism that operatively connects the clamping members relative to the elongated body section/handle section subassembly. Thereafter, clamping action of the clamping members relative to a desired surgical location/region, such as a tumor, may be achieved via extra-corporeal operative control through the trocar port/cannula (step 1112). Once the clamping members have been clamped in a desired fashion, the clamping members may be detached from the elongated body section/handle section subassembly (step 1114), thereby permitting the clamping members to be left in place within the body cavity while the elongated body section/handle section subassembly is removed from the trocar port/cannula, thereby freeing up the trocar port/cannula for introduction of other surgical devices. At the conclusion of the surgical procedure, the clamping members may be removed from the surgical region independent of the trocar port/cannula.

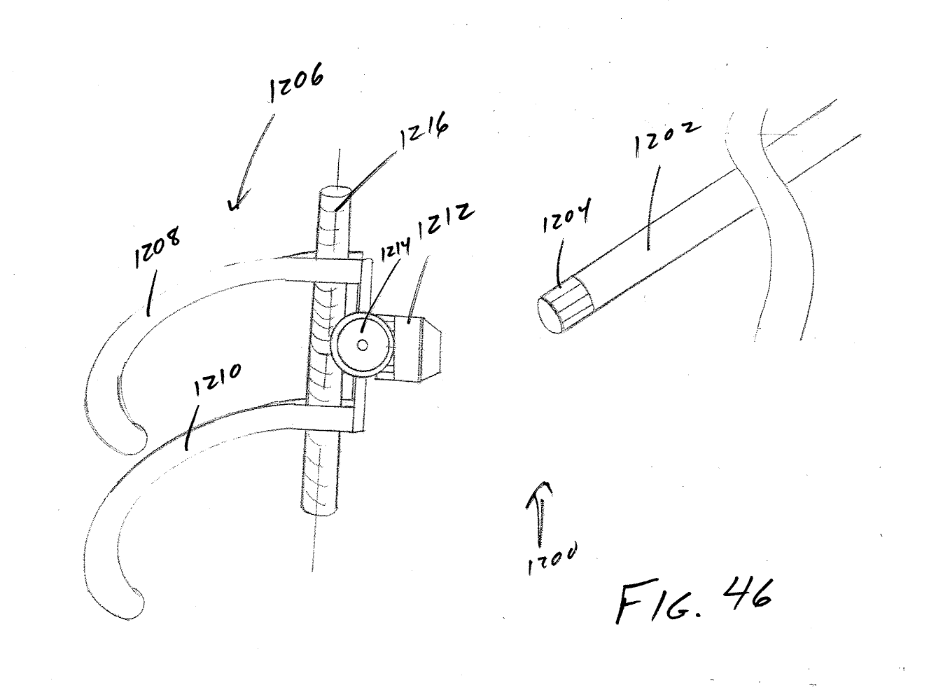

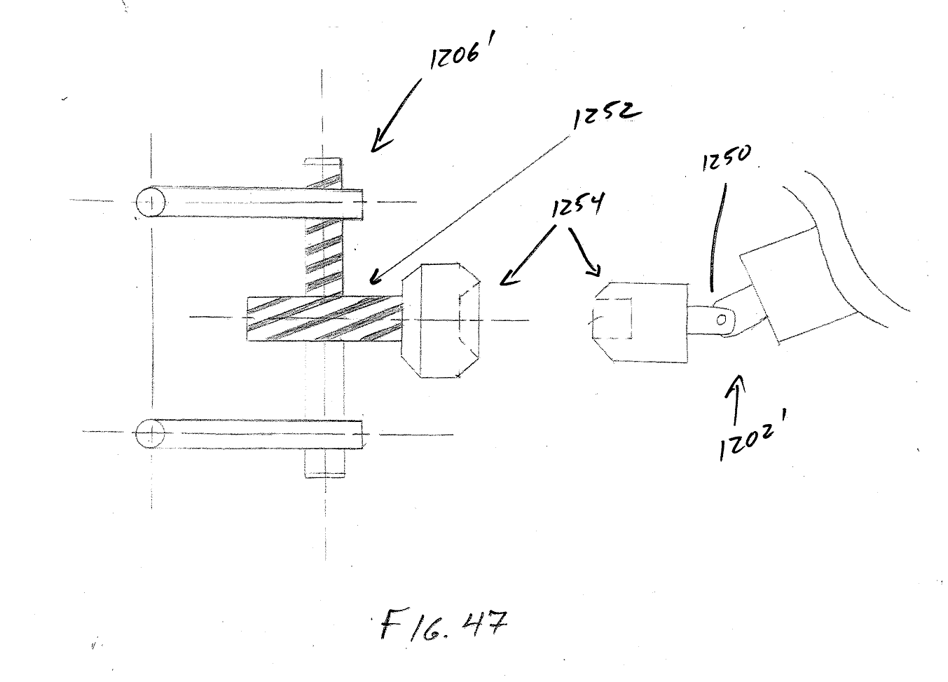

Mechanisms for detachment/reattachment of the clamping section relative to a subassembly defined by the elongated body section and the handle section may be provided to facilitate introduction of the clamping section to the desired clinical location and subsequent operative interaction therewith. As shown in FIGS. 46-47, mating mechanisms may include magnetic functionalities that assist in alignment and mechanical cooperation between an elongated body section of the clamping forceps and a head section of the clamping forceps.

With initial reference to FIG. 46, clamping forceps 1200 includes elongated body section 1202 that defines an operative coupling region 1204 at a distal end thereof. For example, coupling region 1204 may include a multi-faced coupling that is adapted to interact with a corresponding female region of an end effector coupler, as described herein. The elongated body section is typically cylindrical and sized to fit down a trocar port of suitable diameter, e.g., 10 mm (not pictured). An end effector 1206 is generally provided to cooperate with the elongated body section 1202 and functions as a head section with clamping members, as described herein with reference to previous embodiments. The end effector 1206 generally includes first and second clamping members 1208, 1210 that define compound curvature configurations and are configured/dimensioned for clamping approximation. End effector 1206 includes an end effector coupler 1212 that defines a cavity for receipt of coupling region 1204 of elongated body section 1202. To facilitate alignment and coupling of coupler 1212 and coupling region 1204, magnetic functionality may be associated therewith to draw the two components into coupling relationship. Once coupled, the multi-faced coupling geometry of coupling region 1204 cooperates with a corresponding female geometry defined within coupler 1212, thereby establishing cooperative engagement therebetween.

As shown in FIG. 46, once elongated section 1204 is coupled to end effector 1206, rotational motion of elongated section 1204 is translated through a cooperative gear mechanism 1214, 1216 into approximation or separation of clamping members 1208, 1210. In an exemplary implementation, the gear mechanism features a 45° worm gear design with a regular thread associated with a one of the clamping members and a reverse thread associated with the other clamping member. As shown in FIG. 47, a flex coupling 1250 may be associated with the elongated body section 1202′ to provide greater flexibility in relative orientation of the coupling members relative to the elongated body section. In addition, FIG. 47 depicts an intermeshed worm gear mechanism 1252 to facilitate relative movement of the clamping members and a socket-like coupling mechanism 1254 that includes magnetic functionality to effectuate alignment and cooperation between the elongated body section and the end effector. Alternative gear arrangements and/or mechanisms and alternative coupling mechanisms may be employed without departing from the spirit of the present disclosure.

Thus, in exemplary embodiments, a magnetic connection mechanism may be provided to facilitate intra-corporeal detachment/reattachment of the clamping section relative to the elongated body section/handle section subassembly. Gearing mechanisms, e.g., worm gear mechanisms, may be associated with the clamping members, e.g., an end effector subassembly that includes the clamping members, to facilitate approximation of the clamping members through extra-corporeal operative control exercised by the surgeon. The connection mechanism may support rotational functionality, such that the clamping members may be reoriented relative to the desired clinical location/region, e.g., to encircle a tumor or the like.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A clamping forceps, comprising:
a head section including a first clamp member and a second clamp member configured and dimensioned to cooperatively clamp so as to at least partially encircle a tumor or other anatomical structure;
an elongated body section in cooperation with respect to the head section; and
a clamping mechanism at least partially movably mounted with respect to the elongated body section;
wherein actuation of the clamping mechanism maintains substantially parallel actuation of the first and second clamp members with respect to the tumor or the other anatomical structure.

2. The clamping forceps according to claim 1, wherein the clamping mechanism comprises at least one of a cam-style mechanism, a spring-loaded mechanism, a gearing mechanism, a cable wire mechanism, a ratcheted mechanism, a motorized mechanism, a piezoelectric mechanism, a pneumatic mechanism, a solenoid actuator mechanism, a slide-crank mechanism, a slot yoke mechanism, a worm gear mechanism, a scissor mechanism, and a rack and pinion mechanism.

3. The clamping forceps according to claim 2, wherein the clamping mechanism is a scissor mechanism that includes a first scissor arm and a second scissor arm that are each fixed at one end relative to the elongated body section.

4. The clamping forceps according to claim 3, wherein the scissor mechanism comprises a first guide slot and a second guide slot defined with respect to the elongated body section.

5. The clamping forceps according to claim 4, wherein the first scissor arm and the second scissor arm each define an end opposite the end that is fixed relative to the elongated body section, and wherein the opposite ends of the first and second scissor arms are adapted to travel with respect to the first and second guide slots, respectively.

6. The clamping forceps according to claim 1, wherein the clamping mechanism regulates the maximum clamping distance between the first clamp member and the second clamp member, the maximum clamping distance being a distance between clamping surfaces of the first clamp member and the second clamp member.

7. The clamping forceps according to claim 6, wherein the clamping mechanism includes a repositionable backstop for regulating the maximum clamping distance.

8. The clamping forceps according to claim 6, wherein the clamping mechanism regulates the maximum clamping distance based on a length of at least one of the first guide slot and the second guide slot.

9. The clamping forceps according to claim 1, wherein the first and second clamp members define a circular ring configuration.

10. The clamping forceps according to claim 1, wherein the first and second clamp members define a hook-shaped configuration that includes an open section along a perimeter of the first and second clamp members.

11. The clamping forceps according to claim 1, wherein the head section defines a substantially trapezoidal configuration or a compound curvature configuration that facilitates partial encircling of the tumor or other anatomical structure.

12. The clamping forceps according to claim 11, wherein the substantially trapezoidal configuration or the compound curvature configuration are either (i) fixed during fabrication of the head section or (ii) variable and susceptible to reorientation at the time of use.

13. The clamping forceps according to claim 11, wherein an angular transition is defined between the head section and the elongated body section.

14. The clamping forceps according to claim 1, further comprising a handle section, and wherein the elongated body section, the handle section or a combination of the elongated body section and the handle section, includes one or more curved regions.

15. The clamping forceps according to claim 1, wherein the head section is detachable relative to the elongated body section, and further comprising a magnetic mechanism to facilitate reattachment of the head section relative to the elongated body section.

16. A method of clamping with clamping forceps, comprising:
introducing the clamping forceps to a surgical site, the clamping forceps including (i) a first clamp member and a second clamp member configured and dimensioned to at least partially encircle a tumor or other anatomical structure, and (ii) a clamping mechanism;
positioning the first clamp member and the second clamp member so as to at least partially encircle the tumor or the other anatomical structure; and
actuating the clamping mechanism to actuate the first and second clamp members into a clamping position around the tumor or the other anatomical structure in a substantially parallel manner.

17. The method according to claim 16, comprising actuating the first and second clamp members around the tumor or the other structure until a maximum clamping distance between the first clamp member and the second clamp member is reached.

18. The method according to claim 16, comprising actuating a locking mechanism to lock the first clamp member and the second clamp member in a desired clamping position around the tumor or the other structure.

19. The method according to claim 16, wherein the first clamp member and the second clamp member define a head section, wherein the head section is adapted to operatively cooperate with an elongated body section, and wherein introduction of the clamping forceps to the surgical site comprises:
introducing the head section to the surgical site through an incision;
positioning a trocar port or cannula at the surgical site;
introducing at least a portion of the elongated body section to the surgical site through the trocar port or cannula;
intra-corporeally connecting the head section with respect to the elongated body section; and
extra-corporeally actuating the clamping mechanism so as to actuate the first and second clamp members into a clamping position around the tumor or the other anatomical structure.

20. The method according to claim 19, wherein the incision is initially established in connection with positioning of a trocar port or cannula relative to the surgical site.

21. The method according to claim 19, wherein intra-corporeal connection of the head section relative to the elongated body section is facilitated by a magnetic mechanism.

22. The method according to claim 16, wherein the clamping forceps includes a clamping mechanism selected from the group consisting of a cam-style mechanism, a spring-loaded mechanism, a gearing mechanism, a cable wire mechanism, a ratcheted mechanism, a motorized mechanism, a piezoelectric mechanism, a pneumatic mechanism, a solenoid actuator mechanism, a slide-crank mechanism, a slot yoke mechanism, a worm gear mechanism, a scissor mechanism, and a rack and pinion mechanism.

23. The method according to claim 22, wherein the clamping forceps includes an elongated body portion and wherein the clamping mechanism is a scissor mechanism that includes a first scissor arm and a second scissor arm that are each fixed at one end relative to the elongated body section.

24. The method according to claim 23, wherein the scissor mechanism regulates the maximum clamping distance between the first clamp member and the second clamp member based on at least one of (i) a length of at least one of a first guide slot and a second guide slot associated with the scissor mechanism, and (ii) a repositionable backstop adapted to cooperate with at least one of the first guide slot and the second guide slot.

25. The method according to claim 16, wherein the first and second clamp members define at least one of (i) a circular ring configuration, (ii) a hook-shaped configuration that includes an open section along a perimeter of the first and second clamp members, (iii) a substantially trapezoidal configuration, and (iv) a compound curvature configuration, that facilitate partial encircling of the tumor or other anatomical structure.
